(19) United States
(12) Patent Application Publication
(10) Pub. No.: US 2018/0235870 A1
(43) Pub. Date: Aug. 23, 2018

(54) TRANSDERMAL FORMULATIONS FOR DELIVERY OF BERBERINE COMPOUNDS, AND THEIR USE IN THE TREATMENT OF BERBERINE-RESPONSIVE DISEASES AND CONDITIONS

(71) Applicant: DELIVRA INC., Burlington (CA)

(72) Inventors: Joseph Gabriele, Stoney Creek (CA); David Baranowski, Mount Stewart (CA); Beth Buchanan, Charlottetown (CA); Jonathan Zuccolo, Charlottetown (CA); Mikaela Teris, Montreal (CA)

(21) Appl. No.: 15/752,900

(22) PCT Filed: Aug. 17, 2016
(86) PCT No.: PCT/CA2016/050965

§ 371 Date: Feb. 15, 2018

Related U.S. Application Data
(60) Provisional application No. 62/301,318, filed on Feb. 29, 2016.
(60) Provisional application No. 62/205,895, filed on Aug. 17, 2015.

(51) Int. Cl. A61K 9/00; A61K 9/107; A61K 31/4375; A61K 47/10; A61K 47/14; A61K 47/18; A61K 47/26; A61K 47/46; A61P 3/06; A61P 3/10; A61P 35/02

(52) U.S. Cl. CPC A61K 9/0014; A61K 9/107; A61K 31/4375; A61K 47/10; A61K 47/14; A61K 47/183; A61K 31/155; A61K 47/46; A61P 3/06; A61P 3/10; A61P 35/02; A61K 2300/00; A61K 47/26

(57) ABSTRACT

The present application is directed to transdermal formulations for the delivery of berberine compounds to a subject for the treatment of berberine-responsive diseases. In particular, the transdermal formulation comprises: (a) an aqueous phase comprising water and at least one water soluble emulsion stabilizer; (b) an oil phase comprising at least one emulsifier, at least one oil soluble emulsion stabilizer, at least one emollient comprising at least one flavonoid and at least one other emollient; wherein the oil and aqueous phases form an emulsion; (c) an external phase comprising at least one flavonoid containing-extract, at least one phospholipid-complexed flavonoid and at least one source of berberine or analog or derivative thereof; and optionally (d) at least one preservative phase.

### CROSS-REFERENCE TO RELATED APPLICATIONS

This application claims the benefit of U.S. Provisional Patent Application Ser. Nos. 62/205,895, filed Aug. 17, 2015, and 62/301,318, filed Feb. 29, 2016, the contents of which are hereby incorporated by reference in their entirety.

### FIELD

The present application relates to transdermal formulations for effective delivery berberine compounds and various methods of use thereof.

### BACKGROUND

Berberine is a natural health product found in a variety of plant species including barberry (Berberis), the meadow rue (Thalictrum), celandine (Chelidonium), and Goldenseal™ (Hydrastis Canadensis). Berberine contains a permanently charged quaternary amine but is otherwise a non-polar molecule.

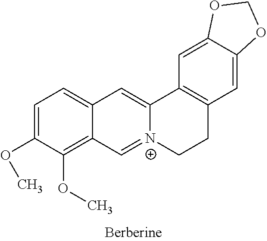 [SMILES: COc1ccc2cc3[n+](cc2c1OC)CCc1cc2c(cc1-3)OCO2]

Berberine's second most common usage is in the textiles industry. Its conjugated tetracyclic skeleton provides its strong yellow color and it has been historically used as a dye (color index of 75160).

Berberine has been shown to have potent glucose-lowering effects (Yao et. al., 2013). The mechanism has not yet been fully elucidated, however there have been studies suggesting multiple action pathways. Berberine also acts by upregulating the expression of the insulin receptor gene in muscle and liver cells, via protein kinase D, to restore insulin sensitivity (Zhang et. al., 2010). As well, berberine inhibits PTP1B, a non-receptor phosphotyrosine protein phosphatase, and promotes the phosphorylation of the insulin receptor, as well as the insulin receptor substrate 1, and AKT (Yao et. al., 2013).

Berberine's most common traditional use is linked to its strong anti-microbial activity against bacteria, viruses, protozoa, fungi, helminthes and Chlamydia (Arayne et. al., 2007). Goldenseal™ extracts are currently listed as a medicinal ingredient within the Health Canada Natural Health Products database for the treatment of infection or inflammation of the digestive tract. At present, the medicinal purposes of Goldenseal™ (and by extension berberine) are limited to oral administration and claims linked to antimicrobial activity.

Clinical research has revealed a multitude of potential applications of berberine in diseases including diabetes, cancer, depression, hypotension, hypocholesterolemia, Alzheimer's disease, cerebral ischemia, and schizophrenia (Singh and Mahajan, 2013). In particular, berberine has shown therapeutic potential as a glucose regulator for the treatment of diabetes. In addition, berberine has been shown to have antioxidant activity, through the inhibition of monoamine oxidase, acetylcholine esterase and butyryl choline esterase as well as lowering the amyloid-3 peptide (Singh and Mahajan, 2013). Thus, berberine has emerged as a natural active with potential applications in a variety of diseases and disease states.

Biological Targets of Berberine

Berberine has several biological targets and has been shown to interact with a variety of proteins, including telomerase, DNA topoisomerase, p53, NF-κB, mitochondrial membrane proteins, and estrogen receptors (Tillhon et al., 2012). Berberine interacts with DNA at specific sequences to form DNA triplexes or G-quadruplexes, and results in the inhibition of telomere elongation, which is relevant in cell cycle and division. Specifically, derivatives of berberine having substituents in position 13 and 9-N-substituted berberines interact with the G-quadruplex to inhibit telomere elongation (Bhadra and Kumar, 2011). Furthermore, berberine itself inhibits DNA topoisomerase I and II activity (Tillhon et al., 2012).

Berberine has been shown to impair cell division. Specifically, studies with berberine have reported cell cycle arrest at the G0/G1 phase in breast cancer MDA-MB 231 and MCF-7 cells, ovarion carcinoma cell lines OVCAR-3 and Skov-2, lung cancer H1299 and A549 cells, human melanoma cell lines WM793 and many others (Tillhon et al., 2012). Berberine also interacts with GADD153, COX-2, MCL-1, and nucleophosmin/B23 and telomerase—all of which play an important role in carcinomas (Tillhon et al., 2012).

Clinical Uses of Berberine

Berberine is a 336.37 dalton molecule that may be obtained from plants or synthesized de novo, having anti-bacterial characteristics and a good safety profile in humans (Yao et al., 2013). Traditional uses of berberine have been to treat bacterial diarrhea in China. Nowadays, studies have illustrated berberine as a potential therapeutic for a variety of diseases and chronic conditions including, for example, diabetes, hyperlipidemia, heart disease, cancer, dyslipidemia and inflammatory disease (Yao et al., 2013).

Administration of Berberine

The oral route of administration for drugs has been used for many natural products, however, many setbacks including low bioavailability, low solubility, low permeability, and side-effects due to first-pass metabolism have provided challenges to effective therapeutic treatments (Vuddanda et al., 2010).

Oral bioavailability is limited by the dissolution of the dosage form, solubility in the gastrointestinal tract, stability and permeability. As the absorption of drugs occurs from the intestinal region into systemic circulation, many of the mechanisms involved include passive transcellular diffusion (for lipophilic drugs), paracellular transport, carrier-mediated transport (for hydrophilic drugs) and endocytosis (Vuddanda et al., 2010). There have been several transporters found to be involved in the drug absorption process, including peptide transporters, amino acid transporters, organic cation transporters, bicarbonate transporters, glucose transporters, neurotransmitter transporters, ion exchangers, salt transporters, urea transporters, folate transporters, fatty acid transporters, nucleoside transporters and ABC transporters, among others. Some of these transporters are also involved in active efflux of drugs and this process affects drug absorption, disposition and elimination (Vuddanda et al., 2010).

Berberine chloride has poor bioavailability of less than 5% with its uptake inhibited by the permeation glycoprotein (P-gp)-mediated efflux. Overcoming bioavailability issues is challenging and may involve optimizing berberine to become a non P-gp substrate, or administration of P-gp inhibitors along with berberine and designing formulations to bypass the efflux pump transport. However, these methods are laborious and time intensive.

In addition, berberine undergoes liver metabolism and hepatobillary excretion, and is a strong antimicrobial that has the potential of killing intestinal microflora upon absorption (Vuddanda et al., 2010). In clinical trials, there has been an abundance of gastrointestinal side-effects reported with the use of oral berberine. In particular, one clinical trial with type 2 diabetes patients, 34.5% of patients experienced adverse gastrointestinal side-effects such as diarrhea and stomach issues during the 13 week berberine treatment (Yin et al., 2008). During the first four weeks of treatment, side effects included diarrhea (n:6; percentage: 10.3%), constipation (4; 6.9%), flatulence (11; 19.0%) and abdominal pain (2; 3.4%). The side effects were only observed for the first four weeks of treatment, thus the investigators decreased the dose of berberine from 0.5 g t.i.d. to 0.3 g t.i.d. (Yin et al., 2008). Gastrointestinal side-effects are not well tolerated among patients and may have significant impact on the compliance and thus the therapeutic efficacy of a berberine composition. Furthermore, berberine is rapidly metabolized and/or poorly absorbed in the gastrointestinal tract, which may be the result of its antimicrobial activity which causes irritation to the gut microflora and thus poorer absorption (Zuo et al., 2006).

In pharmacokinetic experiments using rats, the bioavailability of orally administered berberine is approximately 0.7% (Chen, Miao et al., 2011 and Gong, Chen et al., 2014 and Godugu, Patel et al., 2014). Similarly, low (below 1%) bioavailability in humans has been documented indicating this molecule's extremely poor bioavailability across multiple species (Spinozzi, Colliva et al., 2014). Due to the aforementioned limitations with oral delivery of berberine including bioavailability and the presence of gastrointestinal side-effects, alternate modes of drug delivery are desirable.

Topical formulations of berberine are known, for example, US 2012 0165357, which discloses topical pharmaceutical formulations of berberine and its biologically equivalent analogues, such as palmatine and coptisine, for the treatment of rosacea and other red face-related skin disorders. CN 101152226 discloses a topical preparation of berberine for the treatment of gynecologic diseases and a method of preparing the same. US 2006 0165819 discloses compositions for the treatment of psoriasis and related skin ailments. The composition includes topical skin formulations of glucosamine in combination with berberine in an emollient base. However, these compositions are used by direct application to the area that is affected and do not require absorption of the berberine into blood. In fact, these compositions show poor penetration of active ingredients, poor stability, and increased risk of infection due to altered skin properties and drying of the skin.

The premature metabolism of drugs as a result of the first-pass effect has made transdermal delivery an attractive and alternative strategy (Prausnitz, et al. 2008). For many years, people have placed natural substances on the skin for local ailments. However, lending this strategy towards all therapeutic drugs is not feasible. The human skin acts as a formidable barrier due in large part to the stratum corneum, which mostly consists of a lipid-enriched matrix and blocks entry of most topically applied agents, with the exception of low molecular weight, lipid-soluble drugs. This poses a challenge for administrating medications via the skin for either local cutaneous or systemic therapy.

Transdermal drug delivery strategies have thus focused primarily on the manipulation of this lipid milieu. In particular, penetration enhancers which interact with skin constituents to promote drug transport have provided an approach to increase the range of therapeutic agents that can be delivered.

Despite the significant permeability barrier of the stratum corneum, drug delivery via the skin is a very attractive option and is widely employed for both local and systemic therapy. Topical treatment of cutaneous disorders obviously targets the site of disease, thereby minimizing adverse side effects elsewhere within the body. Delivery of systemic therapies via the skin avoids degradation of the medication within the gastrointestinal tract and first-pass metabolism by the liver, both of which are associated with oral administration of drugs, in addition to evading the pain and safety issues associated with injections. Transdermal delivery of drugs, in some cases, enables infrequent dosing and maintenance of steady state drug levels.

Therefore, it is desirable to provide improved topical therapeutic compositions and delivery systems for the transdermal delivery of berberine and its derivatives across the dermis that could be used as a monotherapy or in conjunction with other agents to treat berberine-responsive diseases and/or conditions.

### SUMMARY

The present application includes transdermal formulations for the delivery of berberine to a subject. In some embodiments, the formulation comprises at least three phases including at least one oil phase, at least one aqueous phase and at least one external phase comprising berberine.

In some embodiments, the present application includes a transdermal formulation comprising:
(a) an aqueous phase comprising water and at least one water soluble emulsion stabilizer;
(b) an oil phase comprising at least one emulsifier, at least one oil soluble emulsion stabilizer, at least one emollient comprising at least one flavonoid and at least one other emollient;
wherein the oil and aqueous phase form an emulsion;
(c) an external phase comprising at least one flavonoid containing-extract, at least one phospholipid-complexed flavonoid and at least one source of berberine or analog or derivative thereof; and optionally
(d) at least one preservative phase.

The present application includes methods for treating one or more berberine-responsive diseases and conditions comprising administering an effective amount of one or more of the transdermal formulations of the application to a subject in need thereof. In some embodiments, the berberine-responsive diseases and conditions are selected from one or more of diabetes, hyperlipidemia, dyslipidemia, heart disease, inflammatory disease, skin disease, metabolic disease, neurological disease and cancer.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 shows a method for solubilizing alkaloid extracts in accordance with the disclosure with a 30-50% recovery of berberine.

FIG. 2 shows a method for solubilizing alkaloid extracts in accordance with the disclosure with a 30-45% recovery of berberine.

FIG. 3 shows a method for solubilizing alkaloid extracts in accordance with the disclosure with a 30-45% recovery of berberine.

FIG. 4 shows a method for solubilizing alkaloid extracts in accordance with the disclosure with a 60% recovery of berberine.

FIG. 5 shows the ¹H NMR spectra of berberine containing extract.

FIG. 6 shows the ¹H NMR spectra of the partially purified berberine containing extract with impurities removed.

FIG. 7 shows the ¹H NMR spectra of the berberine containing extract showing 87% recovery of berberine.

FIG. 8 shows the stability of formulation 3 over 3 months at 45° C. for pH and viscosity evolution.

FIG. 9 shows the stability of formulation 3a over 3 months at 45° C. for pH and viscosity evolution.

FIG. 10 shows the instability of formulation 4 over 3 months at 45° C. for pH and viscosity evolution.

FIG. 11 shows the instability of formulation 6 over 3 months at 45° C. for pH evolution.

FIG. 12 shows the stability of formulation 7 over 3 months at 45° C. for pH and viscosity evolution.

FIG. 13 shows the stability of formulation 8 over 3 months at 45° C. for pH and viscosity evolution.

FIG. 14 is a chromatogram of a serum blood sample demonstrating the presence of berberine in the circulation of an individual following topical treatment with a formulation containing berberine.

FIG. 15 is a ¹H NMR spectrum of dihydroberberine (DHB).

FIG. 16 is a ¹H NMR spectrum of tetrahydroberberine (THB).

FIG. 17 is a UV/VIS spectrum illustrating the effects of ascorbic acid and β-cyclodextrin on the oxidation of DHB to berberine.

FIG. 18 is a western blot analysis of PCSK9 expression in HEPG2 cells treated with vehicle alone, berberine, DHB, or THB. Berberine and DHB down-regulate the expression of PCSK9 whereas THB did not affect expression of PCSK9 as compared to cells alone or vehicle control.

FIG. 19 shows the concentration of berberine in human sera and human plasma after oral and transdermal administrations.

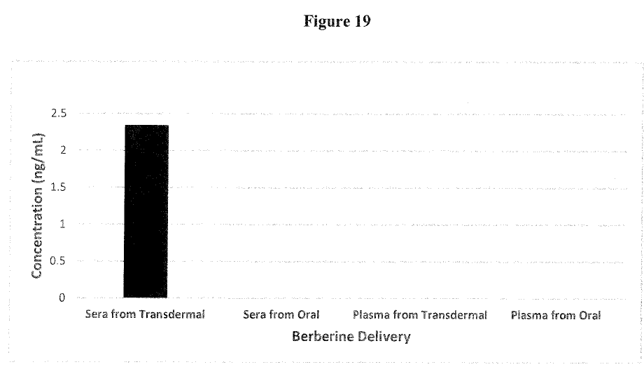

FIG. 20 is a graph showing the circulating levels of berberine in rats following topical administration of exemplary formulations (formulation 3, 3a and 4) disclosed herein.

FIG. 21 is a graph illustrating the pharmacokinetics of berberine through multiple routes of administrations (Oral, PLO, formulation 9).

FIG. 22 is a bar graph showing the calculated concentrations of berberine in formulation 9 and PLO.

FIG. 23 is a graph showing the standard curve for the PCSK9 recombinant protein in sandwich ELISA.

FIG. 24 is a bar graph showing a graphical representation of PCSK9 concentration in serum samples.

FIG. 25 is a line graph showing the change in body weight of Zucker rats treated with vehicle alone, or berberine, simvastatin, and/or metformin.

FIG. 26 is a graph showing the absolute and percent baseline measure on cholesterol by treatment group Zucker rats treated with vehicle alone, or berberine, simvastatin, and/or metformin.

FIG. 27 is a graph showing the absolute and percent baseline measure on triglycerides levels by treatment group Zucker rats treated with vehicle alone, or berberine, simvastatin, and/or metformin.

FIG. 28 is a graph showing the absolute and percent baseline measure on glucose levels by treatment group Zucker rats treated with vehicle alone, or berberine, simvastatin, and/or metformin.

FIG. 29 is a graph showing the absolute and percent baseline measure on HbA1c levels by treatment group Zucker rats treated with vehicle alone, or berberine, simvastatin, and/or metformin.

FIG. 30 is a graph showing the average food intake by treatment group of Zucker rats treated with vehicle alone, or berberine, simvastatin, and/or metformin.

FIG. 31 is a graph showing the average water intake by treatment group of Zucker rats treated with vehicle alone, or berberine, simvastatin, and/or metformin.

FIG. 32 is a graph showing the average body weights by treatment group of Zucker rats treated with vehicle alone, berberine, or combinations with simvastatin, and/or metformin.

FIG. 33 is a graph showing food intake by treatment group of Zucker rats treated with vehicle alone, berberine, or combinations with simvastatin, and/or metformin.

FIG. 34 is a graph showing water intake by treatment group of Zucker rats treated with vehicle alone, berberine, or combinations with simvastatin, and/or metformin.

FIG. 35 is a graph showing glucose levels by treatment group of Zucker rats treated with vehicle alone, berberine, or combinations with simvastatin, and/or metformin.

FIG. 36 is a graph showing HbA1c levels by treatment group of Zucker rats treated with vehicle alone, berberine, or combinations with simvastatin, and/or metformin.

FIG. 37 is a graph showing cholesterol levels by treatment group of Zucker rats treated with vehicle alone, berberine, or combinations with simvastatin, and/or metformin.

FIG. 38 is a graph showing triglyceride levels by treatment group of Zucker rats treated with vehicle alone, berberine, or combinations with simvastatin, and/or metformin.

FIG. 39 is a bar graph of berberine hydrochloride concentrations in plasma of rats subjected to multiple routes of administration.

FIG. 40 is a bar graph showing berberine hydrochloride glucuronide concentrations in plasma of rats subjected to multiple routes of administration.

FIG. 41 is a bar graph showing simvastatin concentrations in plasma of rats subjected to multiple routes of administration.

FIG. 42 is a bar graph showing simvastatin hydroxy acid concentrations concentrations in plasma of rats subjected to multiple routes of administration.

FIG. 43 is an electron micrograph (EM) of a 5% transdermal berberine formulation of the disclosure.

FIG. 44 is an electron micrograph (EM) of a 5% transdermal berberine formulation of the disclosure without tween.

FIG. 45 is an electron micrograph (EM) of a 5% transdermal dihydroberberine formulation of the disclosure.

FIG. 46 is a bar graph showing body weights by treatment group of Zucker rats treated with vehicle alone, berberine, or dihydroberberine.

FIG. 47 is a bar graph showing cholesterol levels by treatment group of Zucker rats treated with vehicle alone, berberine, or dihydroberberine.

FIG. 48 is a bar graph showing triglyceride levels by treatment group of Zucker rats treated with vehicle alone, berberine, or dihydroberberine.

FIG. 49 is a bar graph showing serum berberine levels by treatment group of Zucker rats treated with vehicle alone, berberine, or dihydroberberine.

FIG. 50 is a graph showing a standard series of berberine peak areas.

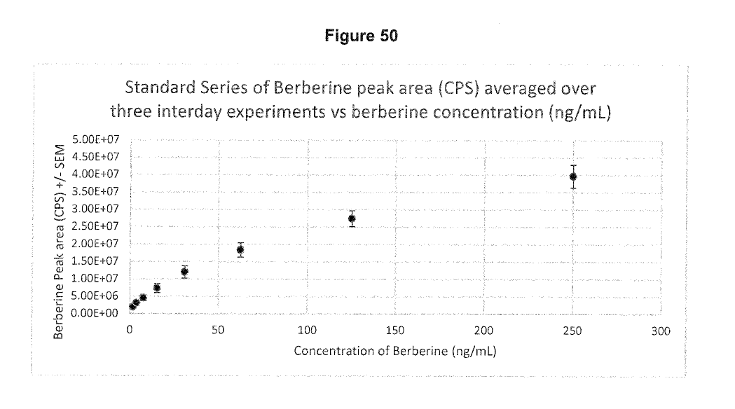

FIG. 51 is a graph showing peak areas of BRB from samples processed from pampa acceptor well (1/20 dilution) over time.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The present description refers to a number of chemical terms and abbreviations used by those skilled in the art. Nevertheless, definitions of selected terms are provided for clarity and consistency.

As used in this application, the singular forms “a”, “an” and “the” include plural references unless the content clearly dictates otherwise. For example, an embodiment including “an agent” should be understood to present certain aspects with one compound or two or more additional compounds.

In embodiments comprising an “additional” or “second” component, such as an additional or second agent, the second component as used herein is chemically different from the other components or first component. A “third” component is different from the other, first, and second components, and further enumerated or “additional” components are similarly different.

The term “agent” as used herein indicates a compound or mixture of compounds that, when added to a formulation, tend to produce a particular effect on the formulation's properties.

The term “thickening agent” as used herein refers to a compound or mixture of compounds that adjusts the thickness of the formulation.

The term “and/or” as used herein means that the listed items are present, or used, individually or in combination. In effect, this term means that “at least one of” or “one or more” of the listed items is used or present.

The term “suitable” as used herein means that the selection of the particular compound or conditions would depend on the specific synthetic manipulation to be performed, and the identity of the molecule(s) to be transformed, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The term “water soluble”, for example as in “water soluble emulsion stabilizer”, refers to a substance that has a solubility in aqueous based solutions that is sufficient for the substance to exert its desired effect at concentrations that are pharmaceutically acceptable.

The term “oil soluble”, for example as in “oil soluble emulsion stabilizer”, refers to a substance that has a solubility in oil based solutions that is sufficient for the substance to exert its desired effect at concentrations that are pharmaceutically acceptable.

“Formulation” and “pharmaceutical formulation” as used herein are equivalent terms referring to a formulation for pharmaceutical use.

The term “pharmaceutically acceptable” means compatible with the treatment of animals, in particular, humans.

The term “effective amount” as used herein means an amount sufficient to achieve the desired result and accordingly will depend on the ingredient and its desired result. Nonetheless, once the desired effect is known, determining the effective amount is within the skill of a person skilled in the art.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e. not worsening) the state of disease, prevention of disease spread, delaying or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. Treatment methods comprise administering to a subject a therapeutically effective amount of an active agent and optionally consists of a single administration, or alternatively comprises a series of applications. The length of the treatment period depends on a variety of factors, such as the severity of the condition, the age of the patient, the concentration of active ingredient or agent, the activity of the compositions described herein, and/or a combination thereof. It will also be appreciated that the effective dosage of the agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for duration sufficient to treat the patient.

“Topical composition” as used herein includes a composition that is suitable for topical application to the skin, nail, mucosa, wound bed or wound cavity. A topical composition may, for example, be used to confer a therapeutic or cosmetic benefit to its user. Specific topical compositions can be used for local, regional, or transdermal application of substances.

The term “topical administration” is used herein to include the delivery of a substance, such as a therapeutically active agent, to the skin or a localized region of the body.

“Transdermal” as used herein includes a process that occurs through the skin. The terms “transdermal,” “percutaneous” and “transcutaneous” can be used interchangeably. In certain embodiments, “transdermal” also includes epicutaneous. Transdermal administration is often applied where systemic delivery of an active is desired, although it may also be useful for delivering an active to tissues underlying the skin with minimal systemic absorption.

“Transdermal application” as used herein includes administration through the skin. Transdermal application can be used for systemic delivery of an active agent; however, it is also useful for delivery of an active agent to tissues underlying the skin with minimal systemic absorption. In certain embodiments, “transdermal application” can also include epicutaneous application.

The term “emollient” as used herein refers to a compound or mixture of compounds that adds or replaces natural oils in the skin, for example by maintaining the integrity of the hydrolipids of the skin.

The term “polar emollient” as used herein refers to emollient compounds, which are generally oils, having heteroatoms that differ in electronegativity. This results in a dipole moment. Typical polar oils are fatty alcohols, esters and triglycerides. While they are still water insoluble and oil-loving, these oils have unique characteristics due to their polar nature. They typically combine with higher hydrophobic lipid balance (HLB) emulsifiers to make stable emulsions, they dissolve materials that are insoluble in nonpolar oils, and they provide unique properties when compared with nonpolar oils such as mineral oil.

The term “medium polar emollient” as used herein refers to emollient compounds, which are generally oils that are less polar than the polar emollients but still more polar than nonpolar oils such as mineral oil.

The term “humectant” as used herein refers to a compound or mixture of compounds intended to increase the water content of the top layers of skin.

The term “emulsifier” of “emulsifying agent” as used herein refers to a compound of mixture of compounds which promote or facilitate the dispersion of one substance in another to form an emulsion.

The term “penetration enhancer” as used herein refers to a compound or mixture of compounds that improves the rate of percutaneous transport of an active agent across the skin for use and delivery of active agents to organisms such as mammals.

The term “flavonoid compounds” as used herein refers to a class of plant secondary metabolites that have the general structure of a 15-carbon skeleton, which contains two phenyl rings (A and B) and heterocyclic ring (C). The basic chemical structure of a flavonoid as used herein is as follows:

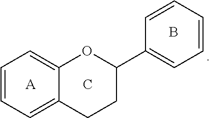

However, the term flavonoid includes the following flavonoids:

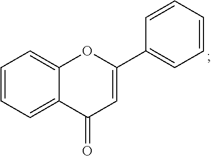 [SMILES: O=c1cc(-c2ccccc2)oc2ccccc12]

isoflavonoids:

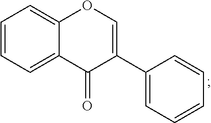 [SMILES: O=c1c(-c2ccccc2)coc2ccccc12]

and
neoflavonoids:

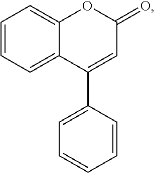 [SMILES: O=c1cc(-c2ccccc2)c2ccccc2o1]

as well as their non-ketone containing counterparts, known as flavanoids. Flavonoids are one of the largest known nutrient families, and include over 6,000 already-identified family members. Some of the best-known flavonoids include rutin, quercetin, kaempferol, catechins, and anthocyanidins. This nutrient group is most famous for its antioxidant and anti-inflammatory health benefits, as well as its contribution of vibrant color to foods.

The term “berberine and its derivatives” as used herein refers to a family of quartenary ammonium salts from the protoberberine group of isoquinoline alkaloids. Berberine salts have the following structure:

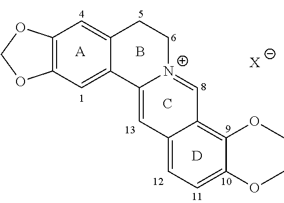

wherein X is a pharmaceutically acceptable anion.

Berberine can be derived from sources of plants which include Berberis aquifolium, Berberis vulgaris, Hydrastis Canadensis, Xanthorhiza simplicissima and Phellodendron amurense californica. The skeleton of berberine is frequently modified, in particular, modifications to the polar C═N⁺ bond on the 8^{th} carbon and the 9-O group with various functional group substitutions have resulted in several pharmacological properties which provide for more selectivity for different therapeutic targets.

The derivatives of berberine can be obtained through chemical modifications of the tetracyclic ring, including reduction of the double bonds in ring C of the berberine skeleton. Reduction of one double bond results in the production of dihydroberberine (DHB) having the following structure:

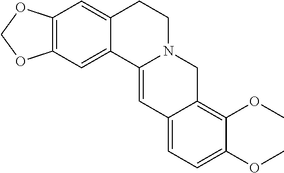 [SMILES: COc1ccc2c(c1OC)CN1CCc3cc4c(cc3C1=C2)OCO4]

DHB is optionally used in the form of a pharmaceutically acceptable salt.

Reduction of two double bonds in ring C produces tetrahydroberberine (THB) having the following structure:

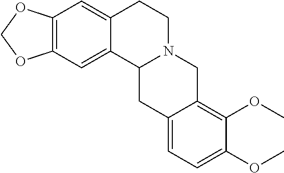 [SMILES: COc1ccc2c(c1OC)CN1CCc3cc4c(cc3C1C2)OCO4]

THB is optionally used in the form of a pharmaceutically acceptable salt.

The chemical derivatives of berberine are also naturally occurring compounds. DHB has been isolated from plants belonging to the genus Glaucidium palmatum (formerly Hydrastis palmatum) and THB was obtained from plants belonging to the genus Hydrastis Canadensis.

The term “pharmaceutically acceptable salt” means an acid addition salt or basic addition salt which is suitable for or compatible with the treatment of subjects, including human subjects.

The term “pharmaceutically acceptable anion” as used herein means organic or inorganic anion formed by the reaction of pharmaceutically acceptable acid with a basic compound. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Such salts may exist in either a hydrated, solvated or substantially anhydrous form. In general, the acid addition salts are more soluble in water and various hydrophilic organic solvents, and generally demonstrate higher melting points in comparison to their free base forms. The selection of the appropriate salt will be known to one skilled in the art. Other non-pharmaceutically acceptable acid addition salts, e.g. oxalates, may be used, for example, in the isolation of the compounds of the invention, for laboratory use, or for subsequent conversion to a pharmaceutically acceptable acid addition salt.

The term “pharmaceutically acceptable basic addition salt” as used herein means any pharmaceutically acceptable organic or inorganic base addition salt of any acid compound. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline or ammonia. The selection of the appropriate salt will be known to a person skilled in the art. Other non-pharmaceutically acceptable basic addition salts, may be used, for example, in the isolation of the compounds for laboratory use, or for subsequent conversion to a pharmaceutically acceptable basic addition salt.

The term “wt %” means a percentage expressed in terms of weight of the ingredient or agent over the total weight of the formulation multiplied by 100.

The term “water” as used herein as an ingredient in the formulations of the application refers to pharmaceutically acceptable water.

### II. Formulations of the Application

In some embodiments, the transdermal formulation base of the present application comprises:
(a) an aqueous phase comprising water and at least one water soluble emulsion stabilizer;
(b) an oil phase comprising at least one emulsifier, at least one oil soluble emulsion stabilizer, at least one emollient comprising at least one flavonoid and at least one other emollient;
wherein the oil and aqueous phase form an emulsion;
(c) an external phase comprising at least one flavonoid containing-extract, at least one phospholipid-complexed flavonoid and at least one source of berberine or analog or derivative thereof; and optionally
(d) at least one preservative phase.

In some embodiments, the transdermal formulation base comprises an oil-in-water emulsion. In some embodiments, the formulation is a multiphase emulsion, such as an oil-in-water-oil emulsion or a water-in-oil-water emulsion.

In other embodiments, the transdermal formulation comprises:

(a) an aqueous phase comprising water, at least one emulsion stabilizer and a humectant;

(b) an oil phase comprising at least one emulsifier, at least one emulsion stabilizer, at least one emollient comprising at least one flavonoid, and at least one other emollient;

wherein the oil and aqueous phase form an emulsion;

(c) an external phase comprising at least one flavonoid containing-extract, at least one phospholipid-complexed flavonoid; and optionally (d) at least one preservative phase; and

(e) a dihydroberberine phase comprising an emulsifier, a surfactant and dihydroberberine.

### Emulsifiers

In some embodiments the emulsifier is any oil-soluble fatty acid ester or mixture of fatty acid esters in which the fatty acid esters have a fatty acid composition similar to the fatty acid composition of skin for generating skin-compatible liquid crystals and to mimic the molecular organization of the intracellular lipidic laminae of the stratum corneum. Such liquid crystals are able to rapidly cross skin layers as well as to integrate into the skin's own lipid barrier to provide strength and greater integrity to this barrier.

In some embodiments the fatty acid esters are selected from sugar alcohol and fatty acid alcohol esters of any C₁₄-C₂₆-fatty acid or mixtures thereof. In some embodiments, the fatty acid esters are esters of fatty acids that are present in olive oil, palm oil and/or canola oil. In some embodiments, the fatty acids are esterified with fatty acid alcohols such as, but not limited to, cetyl alcohol, cetaryl alcohol, lauryl alcohol, stearyl alcholol, myristyl alcohol and/or oleyl alcohol. In some embodiments, the fatty acids are esterified with sugar alcohols such as, but not limited to, sorbitol, glycerol, mannitol, inositol, xylitol, erythritol, threitol, arabitol and/or ribitol. Olive oil fatty acid esters, and their use in transdermal formulations is described, for example, in U.S. Patent Application Publication No. 2011/0021439. In some embodiments, the fatty acid esters are sorbitan esters of palm oil or olive oil, such as sorbitan olivate or sorbitan palmitate. For example, sorbitan olivate is derived from fatty acids present in olive oil and esterified with sorbitol, and sorbitan palmitate is derived from fatty acids present in palm oil and esterified with sorbitol. In other embodiments, the fatty acid esters are cetearyl esters of olive oil, such as cetearyl olivate. For example, cetearyl olivate is derived from fatty acids present in olive oil and esterified with cetearyl alcohol. In further embodiments, the fatty acid esters are cetyl esters of palm oil, such as cetyl palmitate. For example, cetyl palmitate is derived from fatty acid esters present in palm oil and esterified with cetyl alcohol.

In some embodiments, the emulsifier is present in the formulations of the application in an amount of about 1 wt % to about 10 wt %, about 2 wt % to about 8 wt %, or about 4 wt % to about 6 wt %.

### Emulsion Stabilizers

In some embodiments, the emulsion stabilizer is any compound or mixture of compounds that helps to maintain the oil-in-water emulsion. There are three types of emulsion instability: flocculation, coalescence and creaming. Flocculation describes the process by which the dispersed phase comes out of suspension in flakes. Coalescence is another form of instability, which describes when small droplets combine to form progressively larger ones. Emulsions can also undergo creaming, which is the migration of one of the substances to the top or bottom (depending on the relative densities of the two phases) of the emulsion under the influence of buoyancy or centripetal force when a centrifuge is used. Generally, emulsion stability refers to the ability of an emulsion to resist change in its properties over time. In the present application an emulsion stabilizer is present in both the oil phase and the aqueous phase.

In some embodiments, the oil soluble emulsion stabilizer is one or more waxes. In some embodiments the waxes are selected from animal and plant waxes and mixtures thereof. In some embodiments, the plant wax is a wax derived from olives or from palm (e.g. carnauba wax). In some embodiment, the animal wax is beeswax. The one or more waxes are stabilizers that are present in the oil phase of the formulation.

In some embodiment, the oil soluble emulsion stabilizer is present in the formulation in an amount of about 0.5 wt % to about 5 wt %, about 1 wt % to about 4 wt % or about 1 wt % to about 2 wt %.

In some embodiments, the water soluble emulsion stabilizer is one or more thickening agents. In some embodiments, the thickening agents are any compound or mixture of compounds that maintains components in the formulation in suspension and provides a suitable consistency to the formulation.

In some embodiments, the water soluble emulsion stabilizer is selected from natural polymers, gums and synthetic polymers, and mixtures thereof. In some embodiments, natural polymers, gums and synthetic polymers, and mixtures thereof, are water soluble and therefore are present in the aqueous phase of the formulation. In some embodiments, the natural polymers are selected from alginic acid and derivatives thereof, cellulose and derivatives thereof and scleroglucans, and mixtures thereof. In some embodiments, the gums are selected from xanthan gum, tara gum, guar gum and arabic gum, and mixtures thereof. In some embodiments, the synthetic polymers are selected from polyacrylates, polyisobutenes and polysorbates, and mixtures thereof.

In some embodiments, the water soluble emulsion stabilizer is present in the formulations of the application in an amount of about 0.1 wt % to about 1 wt %, about 0.2 wt % to about 0.8 wt %, or about 0.3 wt % to about 0.5 wt %.

### Emollient Comprising at Least One Flavonoid

In some embodiments, the one or more emollients comprising one or more flavonoid compounds are polar emollients. Polar emollients generally include natural oils and extracts from plants. In some embodiments, the polar emollients are derived from fruits (including berries), vegetables, herbs, spices, legumes, leaves, seeds and/or grains. In some embodiments, the polar emollient is a natural oil or extract from citrus, Ginkgo biloba, tea, wine, cacao, onion, kale, parsley, red beans, broccoli, endive, celery, cranberries, blackberries, red raspberries, blackcurrants, acai, blueberries, bilberries, milk thistle, apples, hawthorn, Echinacea, grapes, and/or soy. In some embodiments, the polar emollient is emu oil.

In some embodiments, the polar emollient comprising one or more flavonoid compounds is a natural oil or extract from the genera Rubus, Ribes, Argania, Nymphaea, Peucedanum or Imperatoria, Sambucus, Calendula, Butea, Citrus (e.g. lime), or species or subspecies thereof. In some embodiments, the polar emollient comprising one or more flavonoid compounds comprises Leptospermum Scoparium and/or manuka oil. In some embodiments, the polar emollient comprising one or more flavonoid compounds comprises Argan oil, Sea buckthorn oil, Cicatrol, Protectol, and/or Calendula.

In some embodiments, the emollients comprising one or more flavonoid compounds are present in the formulations of the application in an amount of about 1 wt % to about 20 wt %, about 2 wt % to about 10 wt %, or about 3 wt % to about 5 wt %.

### Further Emollients

The polarity of the emollients used in the present can vary depending on the identity of the emulsifiers and emulsion stabilizers, however can nonetheless be selected by a person skilled in the art. In some embodiments, the formulations of the present application comprise both polar emollients and medium polar emollients.

In some embodiments, further polar emollients used in the present application comprise an oil from an animal in the family Dromaius, for example Dromiceius (emu) or a plant, such as, Jojoba oil, Olive oil and/or coconut oil.

In some embodiments the one or more further polar emollients are present in an amount of about 0.5 wt % to about 10 wt %, about 1 wt % to about 7 wt %, or about 2 wt % to about 5 wt %.

In some embodiments, the medium polar emollient is an ester such as octyl palmitate, isopropyl stearate and isopropyl palmitate, or an alcohol such as octyl dodecanol, or mixtures thereof.

In some embodiments the emollients also act as a thickener (stabilizer) and/or a humectant.

In some embodiments, the one or more medium polar emollients are present in an amount of 0.5 wt % to about 10 wt %, about 1 wt % to about 7 wt %, or about 2 wt % to about 5 wt %.

### Flavonoid-Containing Extract

In some embodiments, the one or more flavonoid-containing extracts water phase is any suitable water soluble natural extract comprising a flavonoid with anti-inflammatory and/or antioxidant properties. In some embodiments, the one or more flavonoid-containing extracts are plant-based extracts, including but not limited to, one or more of Nymphaea caerulea flower extract, Peucedanum ostruthium leaf extract, Sambuscus nigra extract, Calendula flower Extract, Gingko biloba extract, Imperatoria Alpaflor extract, Sambucus Alpaflor extract, Blue lotus extract, Calendula Alpaflor extract, Masterwort extract, Elderberry extract, Angelica extract, green tea extract, chamomile extract, pomegranate pericarp and Peucedanum ostruthium leaf extract.

In some embodiments, the one or more flavonoid-containing extracts for the external phase are present in an amount of about 0.5 wt % to about 10 wt %, about 1 wt % to about 7 wt %, or about 2 wt % to about 5 wt %.

### Phospholipid-Complexed Flavonoid

In some embodiments, the flavonoid in the phospholipid-complexed flavonoid is a bioflavonoid isolated from plants such as, but not limited to, Gingko bilboa, Crataegus sp., Passiflora incarnata, Tormentilla potentilla, Tea sinensis., Aurantium sp., Citrus sp., Eucaliptus sp., Matricaria chamomilla, Rheum sp. and Fagara sylanthoides. In some embodiments, the flavonoid is isolated from green tea, buckwheat, the leaves and petioles of asparagus, fruit of the Fava D-Ante tree, fruits and fruit rinds, for example from citrus fruits such as orange, grapefruit, lemon and lime, and berries such as mulberries and cranberries. In some embodiments, the flavonoid is selected from quercetin, myrcetin, apigenin and rutin, and mixtures thereof.

In some embodiments, the phospholipid is any phospholipid, or mixture of phospholipids, from a plant or animal, or any synthetic phospholipid. In some embodiments, the phospholipid is selected from a phosphatidylcholine, a phosphatidylethanolamine, a phosphatidylinostinol, a phosphatidylserine and lecithin, and mixtures thereof.

In some embodiments, the phospholipid-complexed flavonoid is commercially available. In some embodiments, the phospholipid-complexed flavonoid is prepared by combining the phospholipid and flavonoid in a suitable solvent or mixture of solvents, in a mole ratio of phospholipid:flavonoid of about 0.5 to 2, or about 1, and isolating the resulting complex, for example, but removal of the solvent(s), precipitation and/or lyophilization.

In some embodiments, the phospholipid-complexed flavonoid is present in an amount of about 0.5% wt % to about 5 wt %, about 1 wt % to about 4 wt %, or about 1.5 wt % to about 2.5 wt %.

Complexes of bioflavonoids with phospholipids, their preparation and use, are described, for example in U.S. Pat. No. 5,043,323, the contents of which are incorporated by reference in their entirety.

### Berberine, its Analogs and Derivatives

In some embodiments, the source of berberine and its analogs are alkaloids isolated from plants such as, but not limited to, barberry extract, meadow rue, celandine, Berberis aquifolium, Berberis vulgaris, Hydrastis Canadensis, Xanthorhiza simplicissima, Phellodendron amurense californica and Mahonia aquifolium.

In some embodiments, berberine and its analogs and derivatives are selected from, but not limited to, berberrubine, berberine sulfate, berberine bisulfate, berberine hemisulfate, berberine chloride, jatrorrhizine, palmatine, coptisine, 8-ethyl-12-bromoberberine, 8-ethylberberine, 8-methoxyberberine, 8-methylberberine, 8-n-butyl-12-bromoberberine, 8-n-butylberberine, 8-n-hexyl-12-bromoberberine, 8-n-propyl-12-bromoberberine, 8-n-propylberberine, 8-phenyl-12-bromoberberine, 8-phenylberberine, 9-O-acetylberberrubine, 9-O-benzoylberberrubine, 9-O-ethylberb errubine, 9-O-valerylberberrubine, 9-demethylberberine, 9-demethylpalmatine, 9-O-ethyl-berberrubine, 9-O-ethyl-13-ethylberberrubine, 9-lauroylberberrubine chloride, 12-bromoberrubine, 13-ethoxyb erb erine, 13-ethylberberine, 13-ethylpalmatine, 13-hydroxyb erb erine, 13-methoxyberberine, 13-methylberberine, 13-methylberberrubine, 13-methyldihydroberberine N-methyl salt, 13-methylpalmatine, 13-n-butylberberine, 13-n-butylpalmatine, 13-n-hexylberberine, 13-n-hexylpalmatine, 13-n-propylb erberine, 13-n-propylpalmatine, palmatrubine, dihydroberberines and tetrahydroberberines.

In some embodiments, the source of the berberine or analog or derivative thereof is present in an amount of about 1% wt % to about 20 wt %, about 3 wt % to about 15 wt %, or about 5 wt % to about 10 wt %.

### Water

The balance of the aqueous phase of the composition is made up of water. Further, it is an embodiment that the solvent for the external phase and/or the preservative phase (if present) comprises water. In some embodiments, the water is purified and/or demineralized water. The purified water may, for example, be filtered or sterilized.

In some embodiments, the amount of water in the aqueous phase is about 25 wt % to about 60 wt %, or about 30 wt % to about 55 wt % (based on the total weight of the formulation).

In some embodiments, the amount of water in the external phase is about 0.5 wt % to about 25 wt %, or about 1 wt % to about 20 wt % (based on the total weight of the formulation).

In some embodiments, the amount of water in the preservative phase (if present) is about 0 wt % to about 5 wt %, (based on the total weight of the formulation).

### Preservatives

In some embodiments, the formulations of the present application comprise at least one preservative. Preservatives include antimicrobial agents. In some embodiments the preservatives prevent or inhibit the growth of micro-organisms, including bacteria, yeasts and molds. In some embodiments, the preservatives prevent or inhibit undersirable chemical reactions from occurring.

In some embodiments, the preservative comprises a preservative system comprising phenoxyethanol, benzoic acid, and dehydroacetic acid. In some embodiments, the preservative comprises capryl glycol, which also advantageously has humectant and emollient properties. In some embodiments, the preservative comprises chlorphensin. In some embodiments, the preservative comprises ethylhexylglycerin which also advantageously has skin conditioning and emollient properties and acts as a deodorant. In some embodiments, the preservative comprises a natural antimicrobial agent (antibacterial, antifungal, antiviral). In some embodiments, the natural antimicrobial agent is selected from tea tree oil (Malaleuca alternifolia leaf oil) and myrtyl lemon essential oil. In some embodiments, the preservative comprises a preservative and a preservative booster.

In some embodiments, other components of the formulation have intrinsic anti-microbial properties.

In some embodiments, the one or more preservatives are present in an amount of about 0% wt % to about 5 wt %, about 1 wt % to about 4 wt %, or about 1.5 wt % to about 3 wt %.

### Further Optional Ingredients

In some embodiments, the formulations of the present application further comprise additional ingredients that are common in the transdermal base formulation art. These ingredients are, for example, but not limited to, further active pharmaceutical ingredients, pH adjusters or buffering agents, further solvents, solubilizers, chelating agents, pigments, fragrances, humectants, solubilizers, antioxidants and/or reducing agents.

(a) pH Adjusters/Buffering Agents

In some embodiments, the formulations of the application further comprise one or more pH adjusters, such as acidic, basic, or buffering components. These components may be added to provide the optimal pH balance for the skin. They may also be added to provide an optimal pH for one or more the components of the formulation. In some embodiments the pH of the formulations is adjusted to about 6 to about 7.5.

In some embodiments, the pH adjuster is selected from sodium hydroxide and potassium citrate. In some embodiment, the one or more pH adjusters are present in the formulation in an amount of about 0.05% wt % to about 2.0% wt, about 0.1 wt % to about 1.0 wt %, or about 0.8 wt % to about 0.8 wt %.

In some embodiments, the one or more pH adjusters are in the aqueous phase or the external phase.

(b) Chelating Agents

In some embodiments, the formulations of the application further comprise one or more chelating agents. In some embodiments, the chelating agents bind to metals which can inhibit the activity of the antimicrobial preservatives. In some embodiments, the chelating agent is sodium phytate or ethylendiamine tetraacetic acid (EDTA). In some embodiments, the one or more chelating agents are present in the formulation in an amount of about 0.01% wt % to about 0.2% wt, about 0.02 wt % to about 0.1 wt %, or about 0.03 wt % to about 0.05 wt %.

In some embodiments, the one or more chelating agents are in the aqueous phase or the external phase.

(c) Humectants

In some embodiments, the formulations of the present application further include one or more humectants. In some embodiments, the one or more humectants include, but are not limited to, glycerine (which also acts as an additional solvent).

In some embodiments, the one or more humectants are present in the formulation in an amount of about 0.5 wt % to about 10% wt, about 1 wt % to about 7 wt %, or about 2 wt % to about 5 wt %.

In some embodiments, the one or more humectants are in the aqueous phase.

(d) Solubilizers

In some embodiments, the formulations of the present application further include one or more solubilizers. In some embodiments, the one or more solubilizers include, but are not limited to, inulin lauryl carbamate.

In some embodiments, the one or more solubilizers are present in the formulation in an amount of about 0.01 wt % to about 5% wt, about 0.1 wt % to about 2 wt %, or about 0.2 wt % to about 1 wt %.

In some embodiments, the one or more solubilizers are in the external phase.

(e) Antioxidants

In some embodiments, the formulations of the present application further include one or more antioxidants. In some embodiments, the one or more antioxidants include, but are not limited to, vitamins such as vitamin C, extracted polyphenols and non-essential amino acids.

In some embodiments, the one or more antioxidants are present in the formulation in an amount of about 0.1 wt % to about 10% wt or about 0.5 wt % to about 5 wt %.

In some embodiments, the one or more antioxidants are in the external phase.

(f) Further Active Pharmaceutical Ingredients

In some embodiments, the transdermal formulation of the present application further comprises other active pharmacological ingredients (APIs). As used herein, API may include active molecules derived from natural, synthetic or semi-synthetic means, as well as other active ingredients.

In some embodiments, the formulation further comprises an effective amount of one or more statins, for example, selected from atorvastatin, fluvastatin, lovastatin, pitavastatin, pravastatin, rosuvastatin and simvastatin.

In some embodiments, the formulation further comprises an effective amount of one or more glucose regulating compounds, for example, selected from metformin and glyburide.

In some embodiments, the further active pharmaceutical ingredient (API) is solubilised or dispersed in an effective amount of a suitable vehicle (e.g. solvent(s) or diluent(s)). A skilled person can readily determine which solvents or diluents will be appropriate for a particular API. In some embodiments, the further API is included in an amount of about 0.01 wt % to about 1 wt %, about 0.05 wt % to about 0.5 wt %, or about 0.075 wt %.

(g) Penetration Enhancer

In some embodiments the transdermal formulation of the present application further comprises penetration enhancers known in the art, for example, ethoxydiglycol (transcutanol) and mixtures thereof.

In some embodiments, the penetration enhancer is present in the formulation in an amount of about 0.5 wt % to about 5 wt %, or about 1 wt % to about 2 wt %.

In some embodiments, the transdermal formulation comprises:

(a) an aqueous phase comprising water, at least one emulsion stabilizer (such as xanthan gum) and a humectant (such as glycerine);
(b) an oil phase comprising at least one emulsifier (such as cetearyl olivate, sorbitan olivate), at least one emulsion stabilizer (such as beeswax), at least one emollient comprising at least one flavonoid (such as natural oil or extract of Ribes Nigrum (Black Currant) Seed Oil and/or Rubus Idaeus (Raspberry) Seed Oil), and at least one other emollient (such as isopropyl palmitate);
wherein the oil and aqueous phase form an emulsion;
(c) an external phase comprising at least one flavonoid containing-extract (such as Peucedanum ostruthium leaf extract or Calendula Officinalis Flower Extract), at least one berberine containing extract, at least one phospholipid-complexed flavonoid (such as lecithin and rutin); and optionally

(d) a preservative phase (such as benzoic acid and caprylyl glycol), a solubilizer phase (such as inulin lauryl carbamate), an anti-oxidant phase (such as non-essential amino acids) and thickening phase (glycerine).

In some embodiments, the berberine analog in the transdermal base formulation is dihydroberberine, which upon transdermal absorption through the skin, is re-oxidized to berberine (in vivo). In some embodiments, the dihydroberberine is stable in the transdermal formulations of the disclosure. In one embodiment, dihydroberberine is more hydrophobic than berberine and has increased transdermal absorption in the transdermal formulations. In some embodiments, the transdermal formulation comprises:

(a) an aqueous phase comprising water, at least one emulsion stabilizer (such as xanthan gum) and a humectant (such as glycerine);

(b) an oil phase comprising at least one emulsifier (such as cetearyl olivate, sorbitan olivate), at least one emulsion stabilizer (such as beeswax), at least one emollient comprising at least one flavonoid (such as natural oil or extract of Ribes Nigrum (Black Currant) Seed Oil and/or Rubus Idaeus (Raspberry) Seed Oil), and at least one other emollient (such as isopropyl palmitate);

wherein the oil and aqueous phase form an emulsion;

(c) an external phase comprising at least one flavonoid containing-extract (such as Peucedanum ostruthium leaf extract or Calendula Officinalis Flower Extract), at least one phospholipid-complexed flavonoid (such as lecithin and rutin); and optionally (d) a preservative phase (such as benzoic acid and caprylyl glycol), a solubilizer phase (such as inulin lauryl carbamate), an anti-oxidant phase (such as non-essential amino acids) and thickening phase (glycerine); and

(e) a dihydroberberine phase containing an emulsifier (such as isopropyl myristate) and a surfactant (such as polysorbate 20) and dihydroberberine.

In some embodiments, the formulations of the present application are prepared using a process that comprises:

a) heating an aqueous phase comprising water and at least one water soluble emulsion stabilizer to a first temperature;
(b) heating an oil phase comprising at least one emulsifier, at least one oil soluble emulsion stabilizer, at least one emollient comprising at least one flavonoid, and at least one other emollient to the first temperature;
(c) adding the aqueous phase to the oil phase with stirring at the first temperature and continuing to stir at the first temperature until an emulsion is formed;
(d) cooling the emulsion in (c) to a second temperature; and, in any order:
(e) adding one or more external phases comprising at least one flavonoid containing-extract, at least one phospholipid-complexed flavonoid and at least one source of berberine or analog or derivative thereof to the emulsion at the second temperature; and optionally
(f) adding one or more preservative phases to the emulsion.

In some embodiments, the first temperature is about 65° C. to about 85° C., about 70° C. to about 80° C., or about 75° C.

In some embodiments, the second temperature is about 30° C. to about 50° C., about 35° C. to about 45° C., or about 40° C.

In some embodiments, the formulations of the present application are prepared using a process that comprises:

a) heating an aqueous phase comprising water and at least one water soluble emulsion stabilizer to a first temperature;
(b) heating an oil phase comprising at least one emulsifier, at least one oil soluble emulsion stabilizer, at least one emollient comprising at least one flavonoid, and at least one other emollient to the first temperature;
(c) adding the aqueous phase to the oil phase with stirring at the first temperature and continuing to stir at the first temperature until an emulsion is formed;
(d) cooling the emulsion in (c) to a second temperature; and, in any order:
(e) adding one or more external phases comprising at least one flavonoid containing-extract, at least one phospholipid-complexed flavonoid to the emulsion at the second temperature; and optionally (f) adding one or more preservative phases to the emulsion;
(g) adding to the emulsion a dihydroberberine phase comprising a homogeneous suspension of an emulsifier, a surfactant and dihydroberberine.

In some embodiments, the process further comprises preparing the external phase wherein the at least one phospholipid-complexed flavonoid is stirred with water for a sufficient amount of time to become hydrated prior to being combined with the remaining ingredients for the external phase.

In some embodiments the at least one source of berberine or analog or derivative thereof is combined with at least one antioxidant in a suitable solvent, such as water, propylene glycol and/or an alcohol based solvent prior to being combined with the remaining ingredients for the external phase.

In some embodiments, the phases and emulsions are mixed with a homogenizer prior to combining with other phases.

In some embodiments, the transdermal formulations further comprises an effective amount of one or more statins and/or an effective amount of one or more glucose regulating compounds.

In some embodiments, the phases and emulsions are mixed with a homogenizer prior to combining with other phases.

In some embodiments of the application the formulations described herein are in the form of a cream, gel, liquid suspension, ointment, solution, patch or any other form for transdermal administration and the contents of the formulation adjusted accordingly. In some embodiments, the formulations are in the form of a cream. In some embodiments the cream has a viscosity of about 50000 cps to about 500000 cps, or about 85000 cps to about 200000 cps as measured using a Brookfield RVT T4 2 RPM instrument at room temperature.

The transdermal formulation base can be any such formulation currently used for the topical or transdermal delivery of active agents. Non-limiting examples of such base formulations include, Glaxal base, pluronic lethicin organogel (PLO, Murdan, Sudaxshina in Hospital Pharmacist, July/August 2005, Vol. 12, pp/267-270) etc.

### III. Methods of the Application

In some embodiments, the present application includes a method for transdermal administration of one or more berberine or analog or derivative thereof comprising administering an effective amount of one or more of the formulations of the present application to a subject in need thereof, wherein the one or more formulations comprise the one or more sources of berberine or analog or derivative thereof. In further embodiments, the present application includes a use of one or more formulations of the present application for the administration of one or more berberine or analog or derivative thereof to a subject, wherein the one or more formulations comprise one or more of berberine or analog or derivative thereof.

The present application includes therapeutic methods and uses of the formulations described herein. In some embodiments, the formulations are used in methods to treat one or more berberine-responsive diseases and conditions.

Accordingly, the present application includes methods for treating one or more berberine-responsive diseases and conditions, comprising administering an effective amount of a transdermal formulation of the application to a subject in need thereof. Also included is a use of a transdermal formulation of the application to treat one or more berberine-responsive conditions. In some embodiments the berberine-responsive diseases and conditions are selected from type 1 diabetes, pre-type 1 diabetes, type 2 diabetes, pre-type 2 diabetes, hyperlipidemia, pre-hyperlipidemia, dyslipidemia, heart disease, inflammatory disease, skin disease, metabolic disease, neurological disease and cancer. In some embodiments, the cancer is selected from hepatoma, colon cancer, lung cancer, breast cancer and leukemia. In some embodiments, the transdermal formulations of the application are to treat hyperlipidemia or pre-hyperlipidemia. In some embodiments, the transdermal formulations of the application are to treat type 2 diabetes or pre-type 2 diabetes.

In some embodiments, the present application includes methods for treating hyperlipidemia or pre-hyperlipidemia, comprising administering an effective amount of one or more statins and one or more transdermal formulations of the application to a subject in need thereof.

In some embodiments, the present application includes methods for treating type 2 diabetes or pre-type 2 diabetes comprising administering an effective amount of one or more glucose regulating compounds and one or more transdermal formulations of the application to a subject in need thereof. In some embodiments, the one or more glucose regulating compounds are selected from metformin and glyburide.

In some embodiments, the formulations of the application are used in conjunction with other therapies to treat diseases, conditions or disorders.

### Examples

The following non-limiting examples are illustrative of the present application:

### Example 1: Berberine Partition Coefficient Procedure

The berberine partition coefficient procedure was used to assess the properties of berberine, which was determined to be an overall hydrophilic molecule.

(1) Sample Preparation

10 mg of berberine (Sigma-Aldrich, 663-65-8) was added into a scintillation vial (VWR, VW74504-11). A solution of water saturated octanol was prepared by adding 0.5 mL of distilled water (Biocel Milli-Q water purification system) to 10.5 mL of 1-octanol (Sigma-Aldrich, 297887-1L). The solution was shaken by hand for 30 seconds and allowed to stand for 5 minutes. 10 mL of the water saturated octanol solution was measured and added into the scintillation vial containing berberine and sonicated for two minutes to yield a completely dissolved solution having a homogenous concentration of 1.0 mg/mL. Next, a solution of octanol saturated water solution was prepared by adding 0.5 mL of 1-octanol to 10.5 mL of distilled water. The solution was shaken by hand for 30 seconds and allowed to stand for 5 minutes.

0.7 mL of the 1 mg/mL solution of berberine in water saturated octanol was measured into an HPLC vial (Agilent, 5182-0716). 0.7 mL of the octanol saturated water solution was added into the HPLC vial with berberine in octanol. The solution was mixed for one hour using a rotating tube inverter (VWR, 13916-822). A blank sample containing water and octanol (0.7 mL of each) was prepared.

The samples were centrifuged at 500 rpm for two minutes.

Three control samples were prepared including a blank water sample, blank octanol sample and a berberine stock solution in octanol (1 mg/mL) to be used as negative and positive controls.

(2) Sample Analysis and Instrumentation

The measurement of berberine within the octanol and water layers is completed by reversed-phase chromatography (Agilent 1200 HPLC) with a SB phenyl column (3.5 jam, 2.1×100 mm, Agilent USYF00191) with a guard column. The isocratic running conditions are 30:70 H₂O:MeOH (0.4% AcOH) for ten minutes at a flow rate of 0.25 mL/min with a 10 μL injection volume. The sample was monitored using UV/V is at wavelengths 230/600 nm and 280/600 nm. Autosampler temperature was maintained at 24° C. and column was at 30° C.

The two layers of the sample were injected by adjusting the injection needle height. 0 mm was used for the water layer and +11 mm was used for the octanol layer.

The retention time for berberine elution is 1.4 minutes and peak areas were assigned using the onboard Agilent software based upon absorbance at 230 nm.

(3) Results

A HPLC-UV chromatogram was recorded for each layer of the partition according the procedure described above. The area under the peak between 1.2-2.2 minutes in the chromatogram was used as a measure of concentration of berberine. A linear relationship was assumed to exist between absorbance and concentration of berberine at the concentrations evaluated. The blank water and octanol control samples were injected and the area under the curve between 1.2-2.2 minutes was subtracted from the partition peak area of the same solvent. The area-blank (cps) was used directly in the equation below.

| | Water area | Octanol area |
|---|---|---|
| Sample | (cps) 230 nm | (cps) 230 nm |
| Blank | 354 | 956 |
| Partition | 34696 | 1763 |
| Partition minus blank | 34342 | 807 |

log D=log [(concentration of berberine in octanol layer)/concentration of berberine in water layer)]
log D=log (0.235)
log D=−0.63

Based upon these results, the vast majority of berberine (97%) remains within the water layer as opposed to the lipophilic octanol solvent (˜3%). Furthermore, a log D value of −0.63 was derived, indicative of a hydrophilic compound. Based upon these results, berberine can be considered a hydrophilic compound with relatively high solubility in aqueous solutions in comparison to organic solutions.

### Example 2: Quantification, Purification and Solubility of Berberine from Berberine Containing Extracts

### Quantification of Berberine

Standards

A stock solution of berberine chloride was prepared in water/methanol/acetic acid (50:50:0.1) at a concentration of 1 mg/mL. The stock solution was sonicated for 60 seconds at room temperature until dissolved. This 1 mg/mL stock solution was used to prepare 125 μg/mL solution of berberine by adding 125 μL of 1 mg/mL solution to 875 μL of water/methanol/acetic acid (50:50:0.1). This stock solution was serially diluted to give concentrations of 125 μg/mL, 62.5 μg/mL, 31.2 μg/mL, 15.6 μg/mL, 7.8 μg/mL and 3.9 g/mL. The samples were injected at 10 μL into the HPLC and they were monitored by UV absorbance at 280.20 nm. Peak areas were plotted against berberine concentrations and standard curves in the form of y=Ax+B were calculated using weighted least squares linear regression.

HPLC-UV Instrumentation and Conditions

Isocratic chromatographic separation was performed on a Zorbax Eclipse XDB-C18 column (150×4.6 mm I.D., 5 um particle size, Agilent, S/N USKH009316) using a mobile phase of 68% 30 mM NH₄OAc and 14 mM Et₃N—adjusted to pH 4.85 with glacial acetic acid, 32% Acetonitrile. The flow rate was 1 mL/min, run time was 8 minutes and the injection volume was 10 μL. The column temperature was 30° C. The berberine was analyzed using retention time and absorbance at 280.20 nm.

Sample Preparation for Quantification

The sample of interest was weighed into a scintillation vial in approximately 15 mg and the mass recorded. 1 mL of acetonitrile/water/H₃PO₄ (70/30/0.1) was added per 5 mg of sample. The mixtures were sonicated at room temperature for 5 min in a VWR ultra sonicating cleaner and shaken with a wrist action shaker for 10 min. 100 μL of the solution was transferred by pipette to a 1.7 mL eppendorf tube and centrifuged at 7000 rpm for 2 minutes. The extracts were diluted with 500 μL of a buffered aqueous solution (30 mM NH₄OAc and 14 mM Et₃N; pH—4.85). The diluted extracts were analyzed by LC UV-vis at 280.20 nm using a Zorbax Eclipse XDB-C18 column (150×4.6 mm I.D., 5 m particle size, Agilent, S/N USKH009316) with a C18 guard column (12.5×4.6 mm I.D., Agilent) using a mobile phase of 68% 30 mM NH₄OAc and 14 mM Et₃N; pH—4.85, 32% ACN at a flow rate of 1.00 mL/min over 8.00 min. The sample injection volume was 10 μL and the column temperature was 30° C. Analytical data were acquired and quantification processing was performed by using Analyst software.

Purification of Berberine from Berberine Extracts

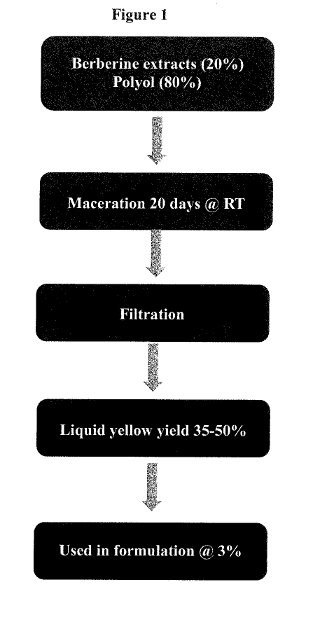

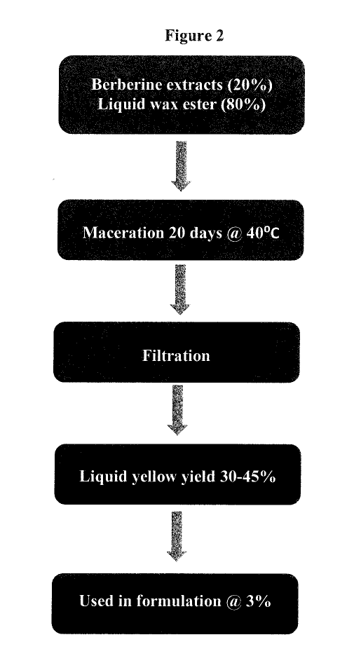

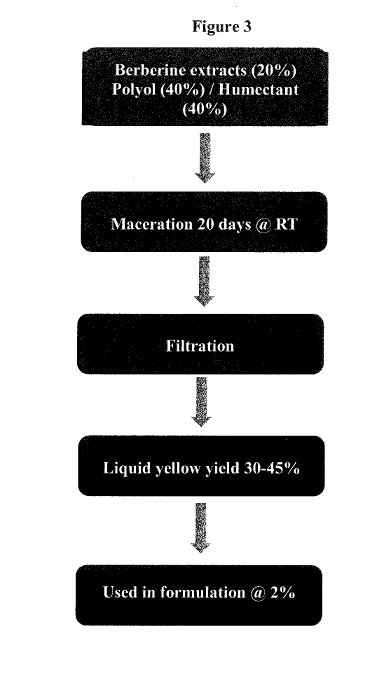

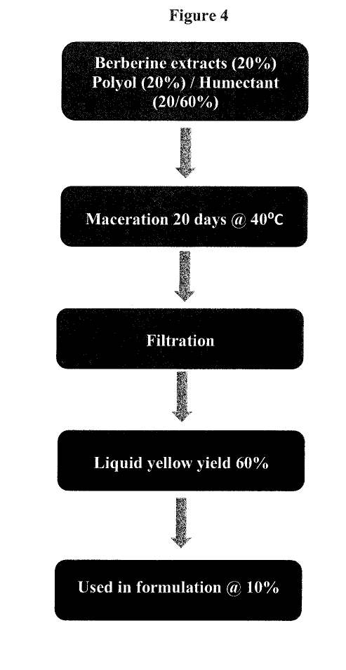

20.0 g of a berberine extract was weighed and placed in a 1 L round bottom flask (RBF) equipped with a stir bar. 5925 mL of 95% ethanol was added to the flask and the mixture was stirred for 20 minutes at room temperature under air. The solution was then filtered, and the solvent removed in vacuo. The sample residue was then weighed. The various solubilization and purification procedures used to isolate berberine from berberine-containing extracts are illustrates from FIGS. 1-4.

### Solubilization of Berberine

Solubility testing of commercial berberine 4:1 and the ethanol extracted berberine (approximately 87% w/w pure).

Exact masses of commercial and purified berberine extract were weighted into labelled scintillation vials and the mass was recorded (Table 1). The indicated solvent was added 200 μL at a time and stirring and sonication were used to assist dissolution. The approximate volumes required to dissolve the sample are summarized in Table 1.

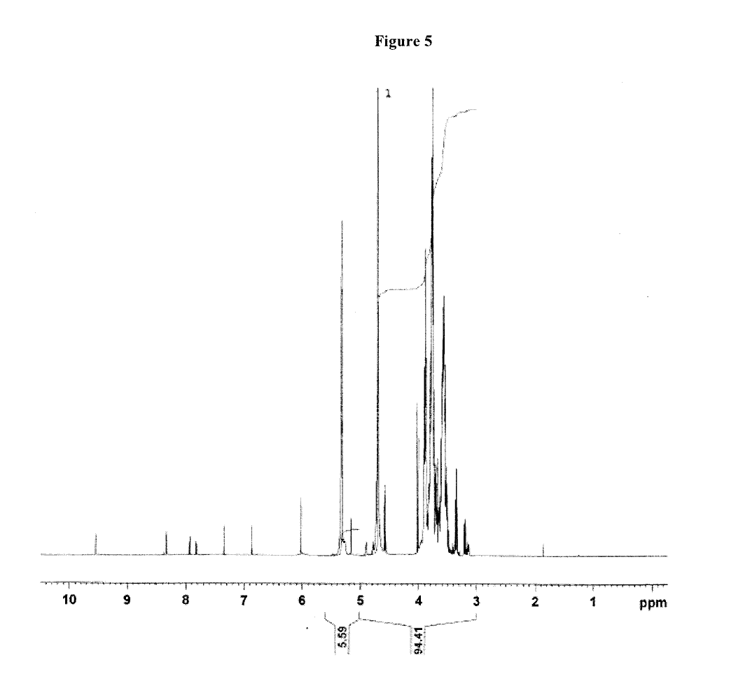

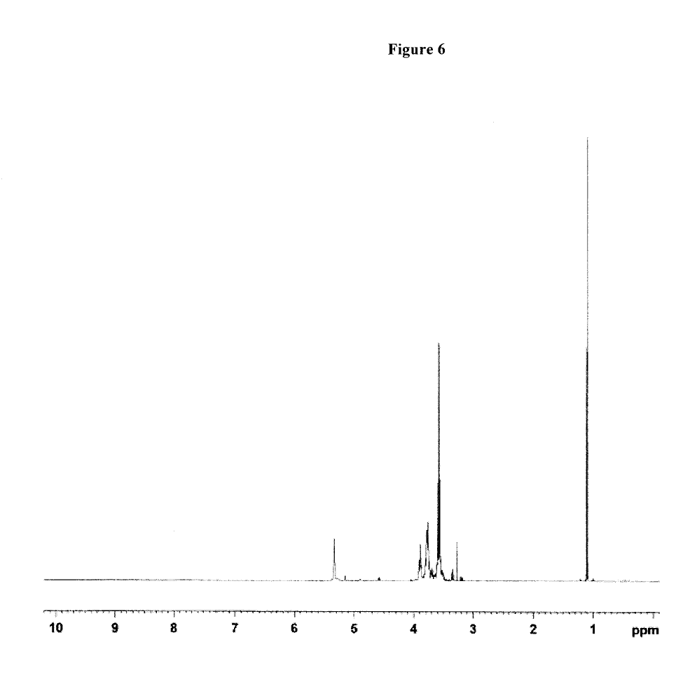

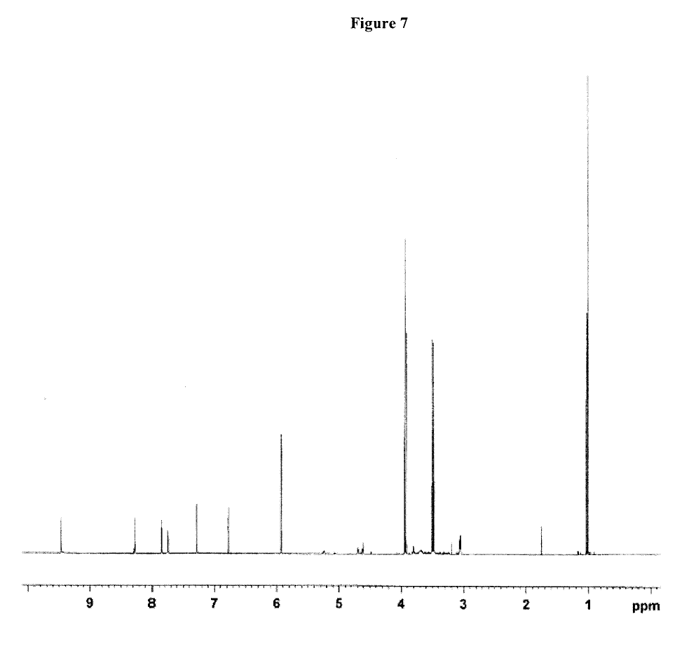

The berberine extracts underwent a maceration procedure prior to formulation development. The maceration procedure may be 20 to 24 days in length and may be performed at room temperature to about 40° C. The yellow liquid that is obtained following a filtration step was used directly in the formulation. ¹H NMR spectrum of the starting material comprising the berberine extract is illustrated in FIG. 5. FIG. 6 shows the ¹H NMR spectra of the berberine extract with impurities removed. FIG. 7 shows the ¹H NMR spectra of the final extract is ˜87% pure berberine.

### Example 3: A Topical Formulation 1 Comprising Berberine Extract

A topical formulation comprising berberine was prepared using the ingredients listed in Table 2. All steps were performed at room temperature.

### Procedure for Making Formulation 1

Step A: In a stainless steel container, the ingredients of Phase A.

Step B: In the main tank, ingredients of Phase B were combined, ensuring the berberine was well dispersed.

Step C: In a stainless steel container, ingredients of Phase C were combined, ensuring the alkaloid was well dispersed.

Step D: In a stainless steel container, the flavonoid-containing ingredients of Phase D were combined.

Step E: In a stainless steel container, preservatives of Phase E were combined.

Step F: In a stainless steel container, ingredients of Phase F were combined.

Step G: In a stainless steel container, ingredients of Phase G were combined.

Step H: In a stainless steel container, ingredients of Phase H were combined.

Step I: Mixtures from steps C-H were added to the mixture from step B. The combined solution mixtures were stirred until homogenous.

Step J: While stirring, thickening agent was added to the solution mixture from step I. The solution mixture was stirred until homogenous.

Step K: While stirring, the mixture of step A was slowly added to the mixture of step J. The solution mixture was stirred until homogeneous.

Step L: While stirring, a surfactant was added from Phase J to the solution mixture from step K. The solution mixture was stirred until homogenous.

### Example 4: A Topical Formulation 2 Comprising Berberine Extract

A topical formulation comprising berberine was prepared using the ingredients listed in Table 3. All steps were performed at room temperature.

### Procedure for Making Formulation 2

Step A: In a stainless steel container, the ingredients of Phase A were combined.

Step B: In the main tank, ingredients of Phase B were combined.

Step C: In a stainless steel container, ingredients of Phase C were combined.

Step D: In a stainless steel container, the flavonoid-containing ingredients of Phase D were combined.

Step E: In a stainless steel container, preservatives of Phase E were combined.

Step F: In a stainless steel container, ingredients of Phase F were combined.

Step G: In a stainless steel container, ingredients of Phase G were combined.

Step H: In a stainless steel container, ingredients of Phase H were combined.

Step I: While stirring, mixtures from steps C-H were added to the mixture from step B. The combined solution mixtures were stirred until homogenous.

Step J: While stirring, thickening agent was added to the solution mixture from step I. The solution mixture was stirred until homogenous.

Step K: While stirring, the mixture of step A was slowly added to the mixture of step J. The solution mixture was stirred until homogeneous.

Step L: While stirring, a surfactant was added from Phase J to the solution mixture from step K. The solution mixture was stirred until homogenous.

### Example 5: A Topical Formulation 3 Comprising Berberine Extract

A topical formulation comprising berberine was prepared using the ingredients listed in Table 4. All steps were performed at room temperature.

### Procedure for Making Formulation 3

Step A: In a stainless steel container, the ingredients of Phase A were combined.

Step B: In the main tank, ingredients of Phase B were combined, ensuring the berberine was well dispersed.

Step C: In a stainless steel container, the flavonoid-containing ingredients of Phase C were combined, ensuring the flavonoid was well dispersed.

Step D: In a stainless steel container, preservatives of Phase D were combined.

Step E: In a stainless steel container, ingredients of Phase E were combined.

Step F: In a stainless steel container, ingredients of Phase F were combined.

Step G: In a stainless steel container, ingredients of Phase G were combined.

Step H: While stirring, mixtures from steps C-G were added to the mixture from step B. The combined solution mixtures were stirred until homogenous.

Step I: While stirring, thickening agent was added to the solution mixture from step H. The solution mixture was stirred until homogenous.

Step J: While stirring, the mixture of step A was slowly added to the mixture of step I. The solution mixture was stirred until homogeneous.

Step K: While stirring, a surfactant was added from Phase J to the solution mixture from step J. The solution mixture was stirred until homogenous.

### Storage Stability of Formulation 3.

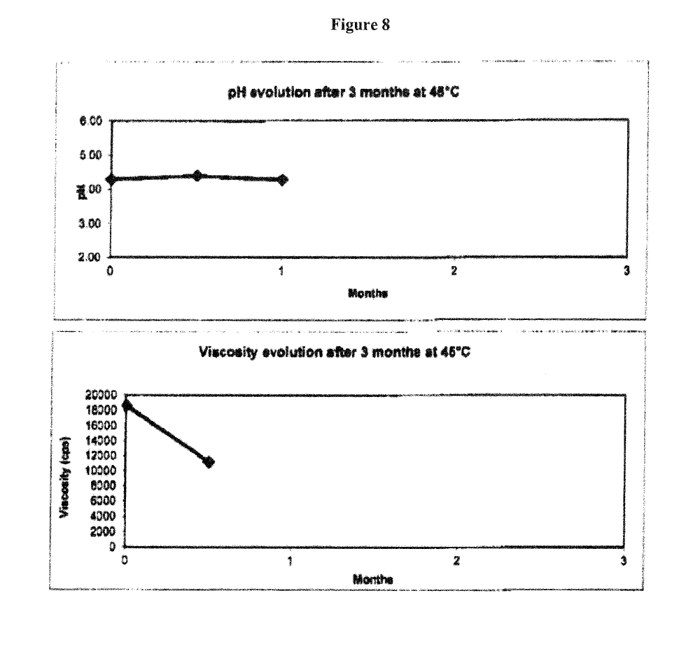

Formulation 3 was evaluated for its stability using four parameter measurements which included pH, texture, color and odour over a period of 3 months at 45° C. Formulation 3 was stable for less than 1 month whereby the formulation provided an average pH evolution of 4.31±0.06 with a consistent viscosity evolution averaging 14,910 cps±5218 as illustrated in FIG. 8. Furthermore, the appearance of the cream produced a yellow color. All measured parameters are illustrated in Table 5.

### Storage Stability of Formulation 3a.

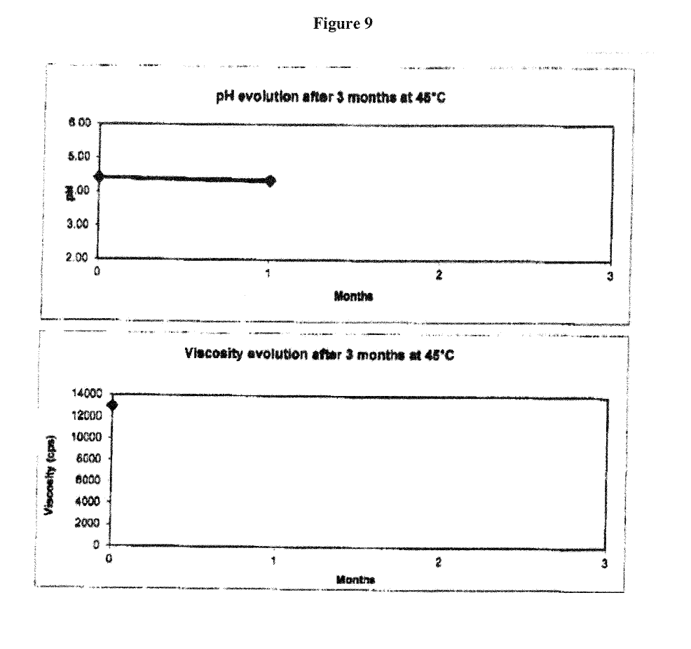

Similarly, a formulation 3a comprising berberine from a different alkaloid extract was evaluated for its stability using four parameter measurements which included pH, texture, color and odour over a period of 3 months at 45° C. Formulation 3a was stable for 1 month whereby the formulation provided an average pH evolution of 4.37±0.04 with a consistent viscosity evolution averaging 12,990 cps as illustrated in FIG. 9. Furthermore, the appearance of the cream produced a yellow color. All measured parameters are illustrated in Table 6.

### Example 6: A Topical Formulation 4 Comprising Berberine Extract

A topical formulation comprising berberine was prepared using the ingredients listed in Table 7. All steps were performed at room temperature.

### Procedure for Making Formulation 4

Step A: In a stainless steel container, the ingredients of Phase A were combined.

Step B: In the main tank, ingredients of Phase B were combined, ensuring the berberine was well dispersed.

Step C: In a stainless steel container, ingredients of Phase C were combined.

Step D: In a stainless steel container, the flavonoid-containing ingredients of Phase D were combined.

Step E: In a stainless steel container, preservatives of Phase E were combined.

Step F: In a stainless steel container, ingredients of Phase F were combined.

Step G: In a stainless steel container, ingredients of Phase G were combined.

Step H: In a stainless steel container, ingredients of Phase H were combined.

Step I: While stirring, mixtures from steps C-H were added to the mixture from step B. The combined solution mixtures were stirred until homogenous.

Step J: While stirring, thickening agent was added to the solution mixture from step I. The solution mixture was stirred until homogenous.

Step K: While stirring, the mixture of step A was slowly added to the mixture of step J. The solution mixture was stirred until homogeneous.

Step L: While stirring, a surfactant was added from Phase J to the solution mixture from step K. The solution mixture was stirred until homogenous.

### Storage Stability of Formulation 4.

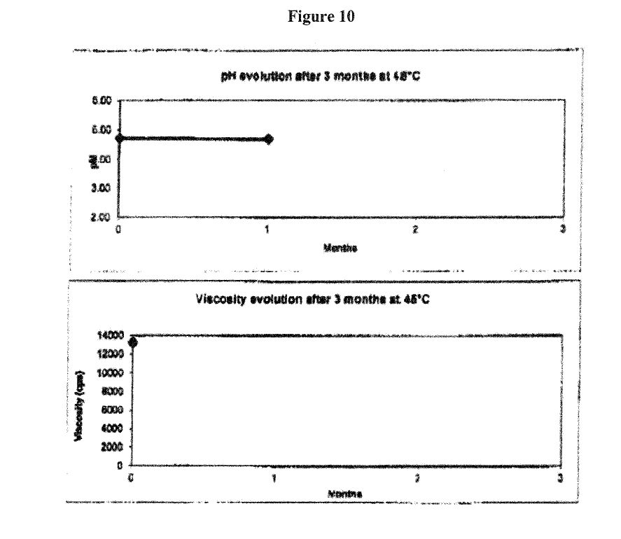

Formulation 4 was evaluated for its stability using four parameter measurements which included pH, texture, color and odour over a period of 3 months at 45° C. Formulation 4 was stable for less than 1 month whereby the formulation provided an average pH evolution of 4.70±0.01 with a consistent viscosity evolution averaging 13,260 cps as illustrated in FIG. 10. Furthermore, the appearance of the cream produced a greenish beige color. All measured parameters are illustrated in Table 8.

### Example 7: A Topical Formulation 5 Comprising Berberine Extract

A topical formulation comprising berberine was prepared using the ingredients listed in Table 9. All steps were performed at room temperature.

### Procedure for Making Formulation 5

Step A: In a stainless steel container, the ingredients of Phase A were combined.

Step B: In the main tank, ingredients of Phase B were combined, ensuring the thickening agent was well dispersed.

Step C: In a stainless steel container, ingredients of Phase C were combined, ensuring the antioxidant and berberine were well dispersed.

Step D: In a stainless steel container, the flavonoid-containing ingredients of Phase D were combined.

Step E: In a stainless steel container, preservatives of Phase E were combined.

Step F: In a stainless steel container, ingredients of Phase F were combined.

Step G: In a stainless steel container, ingredients of Phase G were combined.

Step H: In a stainless steel container, ingredients of Phase H were combined.

Step I: While stirring, mixtures from steps C-H were added to the mixture from step B. The combined solution mixtures were stirred until homogenous.

Step J: While stirring, the thickening agent was added to the solution mixture from step I. The solution mixture was stirred until homogenous.

Step K: While stirring, the mixture of step A was slowly added to the mixture of step J. The solution mixture was stirred until homogeneous.

### Example 8: A Topical Formulation 6 Comprising Berberine Extract

A topical formulation comprising berberine was prepared using the ingredients listed in Table 10.

### Procedure for Making Formulation 6

Step A: In a stainless steel container, the ingredients of Phase A were combined and heated to 75° C.

Step B: In the main tank, ingredients of Phase B were combined and heated to 75° C., ensuring the thickening agent was well dispersed. Once a homogenous solution was achieved, the solution mixture from Step A was added into the main tank, followed by rapid stirring until complete emulsification, about 2-3 minutes. The solution mixture in the main tank was gradually cooled to a reaction temperature of 35-40° C., while stirring.

Step C: In a stainless steel container, ingredients of Phase C were combined, ensuring the antioxidant and berberine were well dispersed.

Step D: In a stainless steel container, the flavonoid-containing ingredients of Phase D were combined.

Step E: In a stainless steel container, preservatives of Phase E were combined.

Step F: In a stainless steel container, ingredients of Phase F were combined.

Step G: In a stainless steel container, ingredients of Phase G were combined.

Step H: In a stainless steel container, ingredients of Phase H were combined.

Step I: While stirring, mixtures from steps C-H were added to the mixture from step B. The combined solution mixtures were stirred until homogenous.

Step J: While stirring, the thickening agent in Phase I was added to the solution mixture from step I. The resulting solution mixture was stirred until homogenous and then cooled to room temperature.

### Storage Stability of Formulation 6.

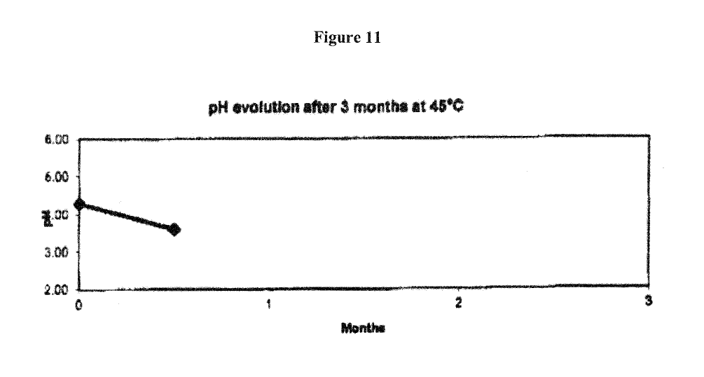

Formulation 6 was evaluated for its stability using four parameter measurements which included pH, texture, color and odour over a period of 3 months at 45° C. Formulation 6 was unstable obtaining only one reading of the parameters at the “0” and “0.5” months mark. The formulation provided an average pH evolution of 3.9±0.49 with an unmeasurable viscosity evolution, as illustrated in FIG. 11. Furthermore, the appearance of the cream was unstable and produced a yellow color. All measured parameters are illustrated in Table 11.

### Example 9: A Topical Formulation 7 Comprising Berberine Extract

A topical formulation comprising berberine was prepared using the ingredients listed in Table 12.

### Procedure for Making Formulation 7

Step A: In a stainless steel container, the ingredients of Phase A were combined and heated to 75° C.

Step B: In the main tank, ingredients of Phase B were combined, ensuring the thickening agent was well dispersed. Once a homogenous solution was achieved, the solution mixture from Step A was added into the main tank, followed by rapid stirring until complete emulsification, about 2-3 minutes. The solution mixture in the main tank was gradually cooled to a reaction temperature of 35-40° C., while stirring.

Step C: In a stainless steel container, ingredients of Phase C were combined, ensuring the berberine was well dispersed.

Step D: In a stainless steel container, the flavonoid-containing ingredients of Phase D were combined.

Step E: In a stainless steel container, preservatives of Phase E were combined.

Step F: In a stainless steel container, ingredients of Phase F were combined.

Step G: In a stainless steel container, ingredients of Phase G were combined.

Step H: In a stainless steel container, ingredients of Phase H were combined.

Step I: While stirring, mixtures from steps C-H were added to the mixture from step B. The combined solution mixtures were stirred until homogenous.

Step J: While stirring, the thickening agent in Phase I was added to the solution mixture from step I. The resulting solution mixture was stirred until homogenous and then cooled to room temperature.

### Storage Stability of Formulation 7.

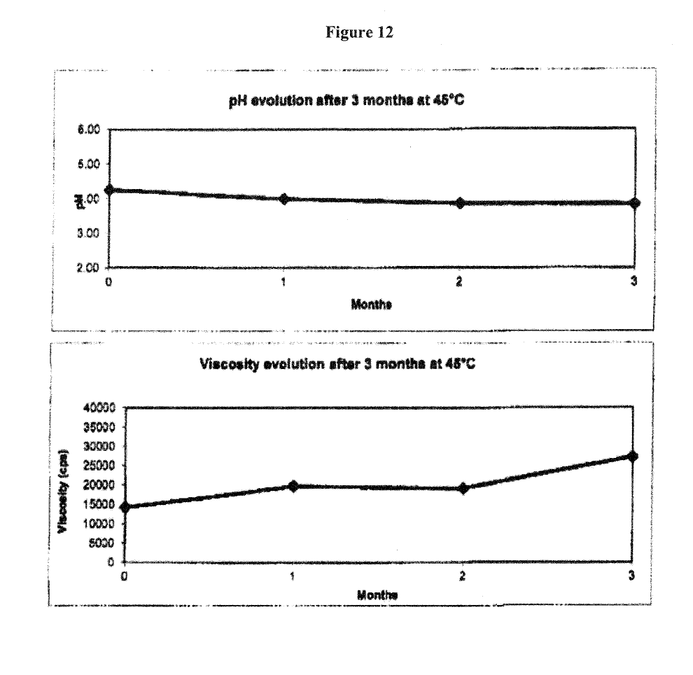

The formulation 7 was evaluated for its stability using four parameter measurements which included pH, texture, color and odour. Formulation 7 maintained its stability in all four parameters measured providing for an average pH evolution of 3.98±0.19 with a consistent viscosity evolution averaging at 20060 cps±5334 as depicted in both FIG. 12 and Table 13.

### Example 10: A Topical Formulation 8 Comprising Tetrahydroberberine Extract

A topical formulation comprising berberine was prepared using the ingredients listed in Table 14.

### Procedure for Making Formulation 8

Step A: In a stainless steel container, the ingredients of Phase A were combined and heated to 75° C.

Step B: In the main tank, ingredients of Phase B were combined, ensuring the thickening agent was well dispersed. Once a homogenous solution was achieved, the solution mixture from Step A was added into the main tank, followed by rapid stirring until complete emulsification, about 2-3 minutes. The solution mixture in the main tank was gradually cooled to a reaction temperature of 35-40° C., while stirring.

Step C: In a stainless steel container, ingredients of Phase C were combined, ensuring the antioxidant and berberine were well dispersed.

Step D: In a stainless steel container, the flavonoid-containing ingredients of Phase D were combined.

Step E: In a stainless steel container, preservatives of Phase E were combined.

Step F: In a stainless steel container, ingredients of Phase F were combined.

Step G: In a stainless steel container, ingredients of Phase G were combined.

Step H: In a stainless steel container, ingredients of Phase H were combined.

Step I: While stirring, mixtures from steps C-H were added to the mixture from step B. The combined solution mixtures were stirred until homogenous.

Step J: While stirring, the thickening agent in Phase I was added to the solution mixture from step I. The resulting solution mixture was stirred until homogenous and then cooled to room temperature.

### Storage Stability of Formulation 8.

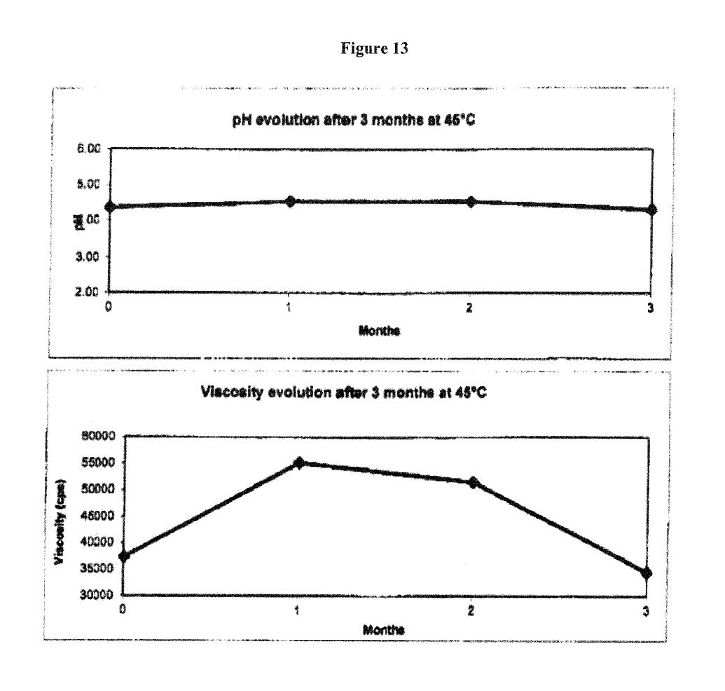

The formulation 8 was evaluated for its stability using four parameter measurements which included pH, texture, color and odour. Formulation 8 maintained its stability in all four parameters measured providing for an average pH evolution of 4.43±0.12 with a consistent viscosity evolution averaging at 44610 cps±10249 as depicted in both FIG. 13 and Table 15.

### Example 11: A Topical Formulation 9 Comprising Berberine Chloride

A topical formulation comprising berberine was prepared using the ingredients listed in Table 16.

### Procedure for Making Formulation 9

Step A: In a stainless steel container, the ingredients of Phase A were combined and heated to 75° C.

Step B: In the main tank, ingredients of Phase B were combined, ensuring the thickening agent was well dispersed. Once a homogenous solution was achieved, the solution mixture from Step A was added into the main tank, followed by rapid stirring until complete emulsification, about 2-3 minutes. The solution mixture in the main tank was gradually cooled to a reaction temperature of 35-40° C., while stirring.

Step C: In a stainless steel container, ingredients of Phase C were combined, ensuring berberine was well dispersed.

Step D: In a stainless steel container, the flavonoid-containing ingredients of Phase D were combined.

Step E: In a stainless steel container, preservatives of Phase E were combined.

Step F: In a stainless steel container, ingredients of Phase F were combined.

Step G: In a stainless steel container, ingredients of Phase G were combined.

Step H: In a stainless steel container, ingredients of Phase H were combined.

Step I: While stirring, mixtures from steps C-H were added to the mixture from step B. The combined solution mixtures were stirred until homogenous.

Step J: While stirring, the thickening agent in Phase I was added to the solution mixture from step I. The resulting solution mixture was stirred until homogenous and then cooled to room temperature.

### Example 12: Berberine Signaling in HEPG2

HEPG2 cells were cultured in 24 well plates to confluence in DMEM supplemented with 10% FBS. After reaching confluence, cells were incubated overnight with 0.1% FBS OPTIMEM for serum starvation. After serum starvation the cells were incubated with berberine, DHB, or THB in 0.1% FBS OPTIMEM using the following dose course: 25 ug/ml, 12.5 ug/ml 6.25 ug/ml 3.125 ug/ml for 24 hours. Supernatant was collected after stimulation and western blotted as follows:

### (1) Resolved on 12.5% gel.

### (2) Transferred to PVDF.

(3) Blocked with 3% BSA TBST
(4) Rabbit anti PCSK9 (Cayman chemicals cat 10007185) at 1/600 in TTBS
(5) Wash 3×5 min with TTBS
(6) HRP-Goat anti-Rabbit 1/1000 (Cayman chemicals)
(7) Wash 3×5 min with TTBS
(8) Detection with ECL

### Example 13: Assessment of Transdermal Delivery of Berberine Formulation 3 in Humans

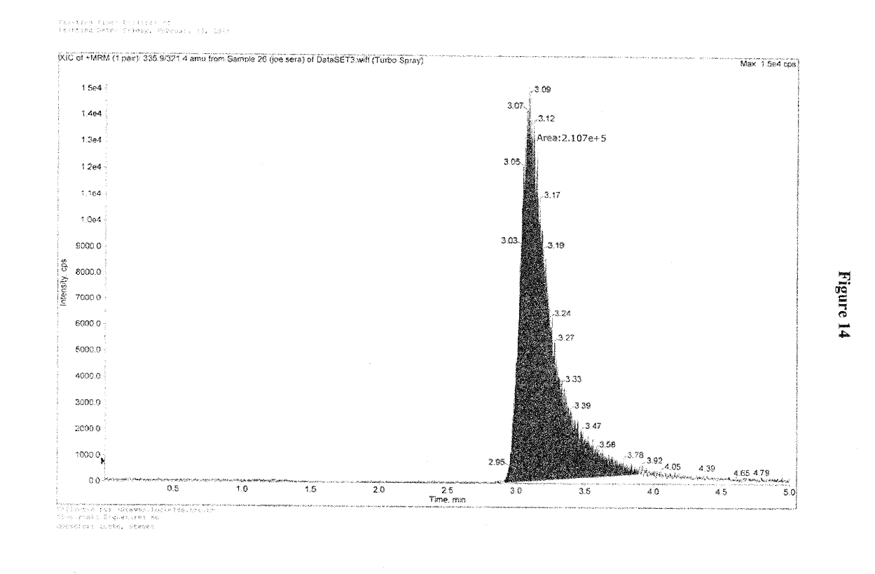

A topical formulation comprising berberine from an alkaloid extract was prepared according to Table 3. This formulation was applied to the forearm of a subject and blood samples were analyzed for berberine content. FIG. 14 shows a chromatogram of a serum blood sample demonstrating the presence of berberine within the circulation of the individual following application of the formulation of Table 3. As FIG. 14 illustrates, berberine was identified in the blood within 15 minutes of topical administration.

Procedure for the Extraction of Berberine from Human Sera

Materials

Berberine chloride (product number CAS 633-65-8) was purchased from Sigma-Aldrich and stored at 10° C. Acetonitrile (HPLC grade, UN1648) was purchased from EMD Millipore. Water (HPLC grade, 8801-7-40) was purchases from Caledon. Glacial acetic acid (reagent grade) was purchased from BioShop. Methanol (reagent grade) was purchased from Omnisolv. Human sera (product number S7023) was purchased from Sigma Aldrich, stored at −80° C. in 1 ml aliquots and thawed immediately prior to use.

LC-MS/MS Instrumentation and Conditions

Gradient chromatographic separation was performed on a Zorbax SB Phenyl column (100×2.1 mm I.D., 3.5 m particle size, Agilent, S/N USYF001191) using a mobile phase of 0.4% acetic acid in water (A) and 0.2% acetic acid in methanol (B) at a flow rate of 0.500 mL/min. The solvent ratio was 30% A/70% B over 5 min and the post run time is 0.1 min. The column temperature was 30° C. and the autosampler temperature was maintained at 4° C. The sample injection volume was 10 μL. A 4000 Q trap from AB Sciex Instruments equipped with electrospray ionization (ESI) was used in the positive ion mode with multiple reaction monitoring (MRM) for the quantitative analysis. Nitrogen was used as the collision gas and the curtain gas. The curtain gas was 10.00 psi, the collision gas was 6 torr, and the ion spray voltage was 4500 volts, the temperature was 350° C., and gas sources 1 and 2 were 14 psi. The declustering potential was 40 volts, the exit potential was 10.00 volts, the focusing lens 1 was −10.50 volts, collision energy was 37.00 volts and the cell exit potential was 4.00 volts. Quantification was performed using the transitions m/z 335.9→321.40 for berberine with a scan time of 100 msec per transition. Analytical data was acquired and quantification processing was performed by using Analyst software.

Unknown Human Serum and Plasma Samples

Separate individuals were given oral berberine (100 mg) or transdermal berberine in formulation 3 (1 g of the formulation containing 100 mg berberine) and blood samples collected 60 min post-administration. For serum processing, filled vacutainers blood collection tubes sat upright after the blood was drawn at room temperature for a minimum of 30 min to allow the clot to form. Samples were centrifuged for 20 min at 1300×g at room temperature. The upper serum was carefully removed and aliquoted in 1.0 mL volumes in eppendorf tubes and frozen at −80° C. All samples were maintained at −80° C. prior to analysis. For plasma processing, filled K-EDTA tubes were gently mixed by inverting the tube 8 to 10 times. Plasma vacutainer tubes were stored upright at 4° C. until centrifugation. Samples were centrifuged for 20 min at 1300×g at room temperature. The upper plasma was carefully removed and aliquoted in 1.0 mL volumes in eppendorf tubes and frozen at −80° C. All samples were maintained at −80° C. prior to analysis.

Preparation of Standard and Quality Control Samples

A stock solution of berberine was prepared in water/methanol/acetic acid (50:50:0.1) at concentration of 1 mg/mL. The stock solution was placed in a VWR ultra sonicating cleaner (model 97049-972) at room temperature for 60 sec at room temperature. This 1 mg/mL stock solution was used to prepared 50 μg/mL solution of berberine by adding 50 μL of 1 mg/mL solution to 950 μL of water/methanol/acetic acid (50:50:0.1). A solution of 1 μg/mL of berberine was prepared by adding 20 μL of a 50 μg/mL solution of meloxicam to 980 μL of water/methanol/acetic acid (50:50:0.1) to give a 50 ng/mL solution of berberine. This stock solution was serially diluted to give concentrations of 50 ng/mL, 25 ng/mL, 12.5 ng/mL, 6.25 ng/mL, 3.125 ng/mL, 1.56 ng/ml, 0.78 ng/mL and 0.39 ng/mL. Peak areas (CPS) were plotted against berberine concentrations and standard curves in the form of y=A+Bx were calculated using weighted least squares linear regression.

Doped Control Samples

95 μL of human sera was spoked with 5 μL of a 1 μg/mL of berberine. 3×100 L duplicates of this sera was aliquotted into 2 mL polypropylene microtubes (MCT-200-C; Catalog no. 311-10-051, Axygen Scientific, Union City, Calif.) and treated with 100 μL of methanol and 100 μL of acetonitrile. The samples were vortexed for 30 seconds (VWR analog vortex mixer) and then centrifuged for 5 minutes at 4000 rpm at room temperature using Eppendorf centrifuge 5430. The organic layer was removed, placed into 1.5 mL polypropylene microtubes (MCT-150-C; Catalog no. 311-08-051, Axygen) and evaporated to dryness (35 minutes) in a Genevac EZ-2 Plus (Fischer Scientific) at 35° C. on the HPLC fraction setting. The residues were re-suspended into a solution of 100 μL of water/methanol/acetic acid (50:50:0.1). The samples were then transferred to HPLC vials (Agilent, product number 5182-0716) and capped with HPLC caps and 10 μL were injected into the LC-MS/MS for analysis.

Blank Control Samples

3×100 μL of Human sera was aliquoted into 2 mL polypropylene microtubes and treated with 100 μL of methanol and 100 μL of acetonitrile. The samples were vortexed for 30 sec and then centrifuged for 5 minutes at 4000 rpm at room temperature using Eppendorf centrifuge 5430. The organic layer was removed, placed into 1.5 mL polypropylene microtubes and evaporated to dryness (35 min) in a Genevac EZ-2 Plus at 35° C. on the HPLC fraction setting. The residues were re-suspended into a solution of 100 μL of water/methanol/acetic acid (50:50:0.1). The samples were then transferred to HPLC vials and capped with HPLC caps and 10 μL was injected into the LC-MS/MS for analysis.

Spiked Control Samples

3×100 μL of Human sera was aliquoted into 2 mL polypropylene microtubes and treated with 100 μL of methanol and 100 μL of acetonitrile. The samples were vortexed for 30 sec and then centrifuged for 5 minutes at 4000 rpm at room temperature using Eppendorf centrifuge 5430. The organic layer was removed, placed into 1.5 mL polypropylene microtubes and evaporated to dryness (35 min) in a Genevac EZ-2 Plus at 35° C. on the HPLC fraction setting. The residues were re-suspended into a solution of 95 μL of water/methanol/acetic acid (50:50:0.1) and 5 μL of 1 μg/mL of berberine. The samples were then transferred to HPLC vials and capped with HPLC caps and 10 μL was injected into the LC-MS/MS for analysis.

Unknown Human Serum and Plasma Samples

100 uL of human serum or plasma was treated with 100 μL of methanol and 100 μL of acetonitrile in 1.7 mL polypropylene microtubes (MCT-175-C, Catalog no. 311-04-051, Axygen Scientific, Union City, Calif.). The samples were vortexed for 30 sec (VWR analog vortex mixer) and then centrifuged for 5 minutes at 4000 rpm at room temperature using Eppendorf centrifuge 5430. The organic layer was removed, placed into 1.7 mL polypropylene microtubes (MCT-175-C, Catalog no. 311-04-051, Axygen Scientific, Union City, Calif.) and evaporated to dryness (35 minutes) in a Genevac EZ-2 Plus (Fischer Scientific) at 35° C. on the HPLC fraction setting. The residues were re-suspended into a solution of 100 μL of water/methanol/acetic acid (50:50:0.1). The samples were then transferred to HPLC vials (product number 5182-0716, Agilent) with inserts (product number 5181-1270, Agilent) and capped with HPLC caps (product number 5182-0720, Agilent) and 10 μL were injected into the LC-MS/MS.

### Example 14: Assessment of Transdermal Delivery of Berberine Formulations in Rodents

Formulations: Three cream based formulations (formulations 3, 3a and 4 of various berberine derived alkaloid extracts were prepared at a final concentration of 10 wt %. All alkaloid extracts underwent a 21-day maceration procedure prior to formulation development.

Materials and Equipment

Berberine chloride (product number CAS 633-65-8) was purchased from Sigma-Aldrich and stored at 10° C. Acetonitrile (HPLC grade, UN1648) was purchased from EMD Millipore. Water (HPLC grade, 8801-7-40) was purchases from Caledon. Glacial acetic acid (reagent grade) was purchased from BioShop. Methanol (reagent grade) was purchased from Omnisolv. Human sera (product number S7023) was purchased from Sigma Aldrich, stored at −80° C. in 1 ml aliquots and thawed immediately prior to use.

Gradient chromatographic separation was performed on a Zorbax SB Phenyl column (100×2.1 mm I.D., 3.5 m particle size, Agilent, S/N USYF001191) using a mobile phase of 0.4% acetic acid in water (A) and 0.2% acetic acid in methanol (B) at a flow rate of 0.500 mL/min. The solvent ratio was 30% A/70% B over 5 min and the post run time is 0.1 min. The column temperature was 30° C. and the autosampler temperature was maintained at 4° C. The sample injection volume was 10 μL. A 4000 Q trap from AB Sciex Instruments equipped with electrospray ionization (ESI) was used in the positive ion mode with multiple reaction monitoring (MRM) for the quantitative analysis. Nitrogen was used as the collision gas and the curtain gas. The curtain gas was 10.00 psi, the collision gas was 6 torr, and the ion spray voltage was 4500 volts, the temperature was 350° C., and gas sources 1 and 2 were 14 psi. The declustering potential was 40 volts, the exit potential was 10.00 volts, the focusing lens 1 was −10.50 volts, collision energy was 37.00 volts and the cell exit potential was 4.00 volts. Quantification was performed using the transitions m/z 335.9-321.40 for berberine with a scan time of 100 msec per transition. Analytical data was acquired and quantification processing was performed by using Analyst software.

Standards and Quality Control Samples

A stock solution of berberine was prepared in water/methanol/acetic acid (50:50:0.1) at concentration of 1 mg/mL. The stock solution was placed in a VWR ultra sonicating cleaner (model 97049-972) at room temperature for 60 sec at room temperature. This 1 mg/mL stock solution was used to prepared 50 μg/mL solution of berberine by adding 50 μL of 1 mg/mL solution to 950 μL of water/methanol/acetic acid (50:50:0.1). A solution of 1 μg/mL of berberine was prepared by adding 20 μL of a 50 μg/mL solution of meloxicam to 980 μL of water/methanol/acetic acid (50:50:0.1). 50 μL of 1 μg/mL was added to 950 μL of water/methanol/acetic acid (50:50:0.1) to give a 50 ng/mL solution of berberine. This stock solution was serially diluted to give concentrations of 50 ng/mL, 25 ng/mL, 12.5 ng/mL, 6.25 ng/mL, 3.125 ng/mL, 1.56 ng/ml, 0.78 ng/mL and 0.39 ng/mL. Peak areas (CPS) were plotted against berberine concentrations and standard curves in the form of y=A+Bx were calculated using weighted least squares linear regression.

Doped Samples

95 μL of human sera was spiked with 5 μL of a 1 μg/mL of berberine. 3×100 L duplicates of this sera was aliquoted into 2 mL polypropylene microtubes and treated with 100 μL of methanol and 100 μL of acetonitrile. The samples were vortexed for 30 seconds (VWR analog vortex mixer) and then centrifuged for 5 minutes at 4000 rpm at room temperature using Eppendorf centrifuge 5430. The organic layer was removed, placed into 1.5 mL polypropylene microtubes (MCT-150-C; Catalog no. 311-08-051, Axygen) and evaporated to dryness (35 minutes) in a Genevac EZ-2 Plus (Fischer Scientific) at 35° C. on the HPLC fraction setting. The residues were re-suspended into a solution of 100 μL of water/methanol/acetic acid (50:50:0.1). The samples were then transferred to HPLC vials (Agilent, product number 5182-0716) and capped with HPLC caps and 10 μL were injected into the LC-MS/MS for analysis.

Blank Samples

3×100 μL of Human sera was aliquoted into 2 mL polypropylene microtubes and treated with 100 μL of methanol and 100 μL of acetonitrile. The samples were vortexed for 30 sec and then centrifuged for 5 minutes at 4000 rpm at room temperature using Eppendorf centrifuge 5430. The organic layer was removed, placed into 1.5 mL polypropylene microtubes and evaporated to dryness (35 min) in a Genevac EZ-2 Plus at 35° C. on the HPLC fraction setting. The residues were re-suspended into a solution of 100 μL of water/methanol/acetic acid (50:50:0.1). The samples were then transferred to HPLC vials and capped with HPLC caps and 10 μL was injected into the LC-MS/MS for analysis.

Spiked Samples

3×100 μL of Human sera was aliquoted into 2 mL polypropylene microtubes and treated with 100 μL of methanol and 100 μL of acetonitrile. The samples were vortexed for 30 sec and then centrifuged for 5 minutes at 4000 rpm at room temperature using Eppendorf centrifuge 5430. The organic layer was removed, placed into 1.5 mL polypropylene microtubes and evaporated to dryness (35 min) in a Genevac EZ-2 Plus at 35° C. on the HPLC fraction setting. The residues were re-suspended into a solution of 95 μL of water/methanol/acetic acid (50:50:0.1) and 5 μL of 1 μg/mL of berberine. The samples were then transferred to HPLC vials and capped with HPLC caps and 10 μL was injected into the LC-MS/MS for analysis.

Unknown Rodent Serum Samples

Three Sprague Dawley rats (each weighing ˜400 grams) each received a different berberine cream and subjects were randomly selected for a treatment condition. Following the collection of a baseline blood sample, each rate received 0.5 grams of the designated cream to an area of the skin of the back, following shaving. Blood samples were collected 2 hours post administration. An additional 0.5 gram dose of the designated cream was re-applied to each subject immediately following the 2 hour blood collection. Subsequent blood collections were conducted at the 4 hour and 6 hour time points following the first dose. At each collection time point, blood was collected into SST tubes and processed using standing operation procedures to yield ˜200 μL of serum. On completion of the blood collection procedures, all three were euthanized according to standard operating procedures. Serum samples from each animal and time point were catalogued and maintained on dry ice or at −80° C. until processed for berberine concentrations.

For each time point and animal, 100 μL of serum was treated with 100 μL of methanol and 100 μL of acetonitrile. The samples were vortexed for 30 sec and then centrifuged for 5 minutes at 4000 rpm at room temperature using Eppendorf centrifuge 5430. The organic layer was removed, placed into 1.5 mL polypropylene microtubes and evaporated to dryness (35 minutes) in a Genevac EZ-2 Plus at 35° C. on the HPLC fraction setting. The residues were re-suspended into a solution of 100 μL of water/methanol/acetic acid (50:50:0.1). The samples were then transferred to HPLC vials and capped with HPLC caps and 10 μL was injected into the LC-MS/MS for analysis.

### Example 15: Oral and Transdermal Berberine Bioavailability in Rat Serum Using Exemplary Formulation 9 and PLO

Animal Administration and Manipulations

All in vivo animal husbandry, treatment regiments and sample collections were completed by InterVivo, a contract research organization. The protocol used in the study was reviewed by an internal animal ethics review board. The study was completed using Sprague-Dawley rats (Charles River) at three weeks of age. A rectangle of hair (that totaled approximately 10% of the skin surface area) was marked on the mid to lower back. The hair from this area was clipped using peanut clippers when animals were anaesthetized for catheter placement. All blood samples were collected from the carotid artery catheter. The application area was marked with permanent marker as a guide. At the time of dosing, the test articles were applied in a thin, uniform layer covering a target 10% of body surface area. Following application, the test article was held in contact with the skin and protected from removal by the animal with a Vet Wrap bandage. On the day of dosing, 1 gram aliquots of the test article were weighed out. Test article was applied to the shaved area using a spatula. Vetrap (VWR) was then placed at the site of dermal application and wrapped around the torso of the animal. This “harness” limited any transfer of the product and prevented oral ingestion.

A total of three animals were orally administered a 50 mg bolus of berberine hydrochloride (Sigma Aldrich) as a suspension in 0.1% (w/w) methylcellulose. A total of eight animals were transdermally administered berberine that was formulated in an exemplary formulation as described in Table 16, at a concentration of 5% (w/w) with a final exposure level of 50 mg. A total of eight animals were transdermally administered berberine that was formulated in commercial poly-lecithin organogel (PLO) at concentrations of 5% (w/w) with a final exposure level of 50 mg.

From each animal, blood samples were collected at the 0, 0.5, 1, 1.5, 2, 3, 4 and 5 hour time points post-administration via carotid artery catheter. Whole blood was collected into serum separator tubes with 100 μL serum per time point frozen at −80° C. until quantified.

Preparation of Formulations

The berberine hydrochloride used in this study was purchased from Sigma-Aldrich and the same lot was used for oral administration and preparation of the transdermal formulas. The 5% (w/w) berberine in an exemplary formulation 9 of table 16 and 5% (w/w) berberine in PLO was prepared by MNK Recherches (Montreal, QC).

Blinding

Serum samples provided by the contract research organization were labelled with 4-5 character unique identifiers by the research supervisor. Technical researchers completed the quantification procedure using the unique identifiers and submitted data using the unique identifiers. Subsequent to the complete quantification of all samples, the data was unblended and results generated.

Preparation of Standard Series

2.0-5.0 mg of powdered berberine hydrochloride (Sigma Aldrich B3251) was weighed into a scintillation vial and the mass was recorded. The recorded mass was then used to calculate the volume of methanol/water (50:50) needed to generate a final concentration of 1 mg/mL and that amount was accurately added to the vial using a pipette. The solution was vortexed for 30 seconds or until the berberine was completely dissolved.

A 50 μg/mL solution of berberine hydrochloride was prepared by addition 50 μL of 1 mg/mL stock solution to 950 μL of methanol/water (50:50) and the resulting solution was vortexed for 10 seconds. A 1 μg/mL solution of berberine hydrochloride was prepared by adding 20 μL of 50 μg/mL solution to 980 μL of methanol/water (50:50) and the resulting solution was vortexed for 10 seconds.

A 1 mg/mL solution of chelerythrine chloride (internal standard—Sigma Aldrich C2932) was prepared in 50:50 methanol/water. A 20 μg/mL solution of chelerythrine chloride was prepared by adding 20 μL of the 1 mg/mL solution of chelerythrine chloride to 980 μL 50:50 methanol/water and the resulting solution was vortexed for 10 seconds. A 1 μg/mL solution of chelerythrine chloride was prepared by adding 50 μL of 20 μg/mL solution of chelerythrine chloride to 950 μL 50:50 methanol/water and the resulting solution was vortexed for 10 seconds.

A 200 ng/mL solution of berberine hydrochloride+5 ng/mL solution of chelerythrine chloride was prepared by adding 200 μL of 1 μg/mL solution of berberine hydrochloride and 5 μL of 1 μg/mL solution of chelerythrine chloride to 795 μL of methanol/water (50:50) (Solution 1) and the resulting solution was vortexed for 10 seconds. A 5 ng/mL solution of chelerythrine chloride was prepared by adding 40 μL of 1 μg/mL solution of chelerythrine chloride to 7.960 mL of methanol/water (50:50) and the resulting solution was vortexed for 10 seconds (Solution 2). Solution 1 was serial diluted with solution 2 (100 μL) in HPLC vials with inserts to give a standard series with concentrations of 200, 100, 50, 25, 12.5, 6.25, 3.125, 1.56, 0.78, and 0.39 ng/mL of berberine and a constant concentration of 5 ng/mL solution of chelerythrine.

Ratio of peak area of berberine to peak area of chelerythrine chloride was plotted against berberine concentrations and used to produce a set of standard curves in the form of y=A+Bx using weighted least squares linear regression.

Doped Samples

Doped Serum—250 ng/mL

500 μL of the 50 μg/mL berberine hydrochloride solution was added to 500 μL of methanol/water to give a 25 μg/mL solution, and the resulting solution was vortexed for 10 seconds. 5 μL of the 25 μg/mL solution of berberine was added to 495 μL of human sera in a 1.7 mL of polypropylene microtube and the tube was vortexed for 10 seconds.

40 μL of the doped serum was pipetted into each of 3, 1.7 mL polypropylene microtubes. 200 μL of acetonitrile (3% acetic acid) was added to each Eppendorf tube containing doped serum and the samples were then vortexed for 2 minutes and then centrifuged for 10 min at 11,000 rpm at room temperature using Eppendorf centrifuge.

The supernatant was transferred into a clean labelled 1.7 mL polypropylene microtube and evaporated to dryness (three hours) in a Genevac EZ-2 Plus at 35° C. on the medium boiling point setting. The samples were placed in the freezer (−20° C.) overnight. The following morning, the samples were allowed to come to room temperature before proceeding with the next step. The residues were re-suspended in 80 μL solution 2 and vortexed for 2 minutes then centrifuged for 2 minutes at 11,000 rpm at room temperature using Eppendorf centrifuge.

The solution was carefully transferred into HPLC vials with inserts and capped with HPLC caps wherein 25 μL was injected to the LCMS for analysis.

Dope −25 ng/mL

50 μL of the 50 μg/mL berberine hydrochloride solution was added to 950 μL of methanol:water to give a 2.5 μg/mL solution, and the resulting solution was vortexed for 10 seconds. 5 μL of the 2.5 μg/mL solution of berberine was added to 495 μL of human sera in a 1.7 mL polypropylene microtube and the tube was vortexed for 10 seconds.

40 μL of the doped serum was pipetted into each of 3, 1.7 mL polypropylene microtube. 200 μL of acetonitrile (3% acetic acid) was added to each Eppendorf tube containing doped serum and the samples were then vortexed for 2 minutes and then centrifuged for 10 minutes at 11,000 rpm at room temperature using Eppendorf centrifuge.

The supernatant was transferred into a clean labelled 1.7 mL polypropylene microtube and evaporated to dryness (three hours) in a Genevac EZ-2 Plus at 35° C. on the medium boiling point setting. The samples were placed in the freezer (−20° C.) overnight. The following morning, the samples were allowed to come to room temperature before proceeding with the next step.

The residues were re-suspended in 80 μL solution 2 and vortexed for 2 minutes then centrifuged for 2 minutes at 11,000 rpm at room temperature using Eppendorf centrifuge.

The solution was carefully transferred into HPLC vials with inserts and capped with HPLC caps and 25 μL was injected to the LCMS for analysis.

Dope −2.5 ng/mL

5 μL of a 50 μg/mL berberine hydrochloride solution was added to 995 μL of methanol/water to give a 0.25 μg/mL solution, and the resulting solution was vortexed for 10 seconds. 5 μL of the 0.25 μg/mL solution of berberine was added to 495 μL of human sera in a 1.7 mL polypropylene microtube and the tube was vortexed for 10 seconds.

40 μL of the doped serum was pipetted into each of 3, 1.7 mL polypropylene microtubes. 200 μL of acetonitrile (3% acetic acid) was added to each Eppendorf tube containing doped serum and the samples were then vortexed for 2 minutes and then centrifuged for 10 minutes at 11,000 rpm at room temperature using an Eppendorf centrifuge.

The supernatant was transferred from the Eppendorf tube and placed into a clean labelled 1.7 mL polypropylene microtube and evaporated for dryness (three hours) in a Genevac EZ-2 Plus at 35° C. on the medium boiling point setting. The samples were placed in the freezer (−20° C.) overnight. The following morning, the samples were allowed to come to room temperature before proceeding with the next step.

The residues were re-suspended in 80 μL of solution 2 and vortexed for 2 minutes then centrifuged for 2 minutes at 11,000 rpm at room temperature using Eppendorf centrifuge.

The solution was carefully transferred to HPLC vials with inserts and capped with HPLC caps and 25 μL was injected to the LCMS for analysis.

Spiked Samples

Spiked Samples—25 μg/mL

40 μL of blank serum was pipetted into each of 3, 1.7 mL polypropylene microtube. 200 μL of acetonitrile (3% acetic acid) was added to each Eppendorf tube containing serum and the samples were then vortexed for 2 minutes and then centrifuged for 10 minutes at 11,000 rpm at room temperature using an Eppendorf centrifuge.

The supernatant was transferred from the Eppendorf tube and placed into a clean labelled 1.7 mL polypropylene microtube and evaporated for dryness (three hours) in a Genevac EZ-2 Plus at 35° C. on the medium boiling point setting. The samples were placed in the freezer (−20° C.) overnight. The following morning, the samples were allowed to come to room temperature before proceeding with the next step.

The residues were re-suspended in 80 μL of solution 3 and vortexed for 2 minutes then centrifuged for 2 minutes at 11,000 rpm at room temperature using Eppendorf centrifuge. Solution 3—a 25 ng/mL solution of berberine+5 ng/mL solution of internal standard (made by adding 25 μL of 1 μg/mL solution of berberine chloride and 5 μL of 1 μg/mL solution of internal standard to 970 μL 50:50 methanol/water, and vortexed for 10 seconds.

The solution was carefully transferred to HPLC vials with inserts and capped with HPLC caps and 25 μL was injected to the LCMS for analysis.

Blank Samples

40 μL of blank serum was pipetted into each of 3, 1.7 mL polypropylene microtube. 200 μL of acetonitrile (3% acetic acid) was added to each Eppendorf tube containing serum and the samples were then vortexed for 2 minutes and then centrifuged for 10 minutes at 11,000 rpm at room temperature using an Eppendorf centrifuge.

The supernatant was transferred from the Eppendorf tube and placed into a clean labelled 1.7 mL polypropylene microtube and evaporated for dryness (three hours) in a Genevac EZ-2 Plus at 35° C. on the medium boiling point setting. The samples were placed in the freezer (−20° C.) overnight. The following morning, the samples were allowed to come to room temperature before proceeding with the next step.

The residues were re-suspended in 80 μL of solution 2 and vortexed for 2 minutes then centrifuged for 2 minutes at 11,000 rpm at room temperature using Eppendorf centrifuge.

The solution was carefully transferred to HPLC vials with inserts and capped with HPLC caps and 25 μL was injected to the LCMS for analysis.

Unknown Samples

Unknown serum was thawed and vortexed for 10 seconds. 40 μL of the unknown serum was pipetted into each of 3, 1.7 mL of polypropylene microtubes. 200 μL of acetonitrile (3% acetic acid) was added to each Eppendorf tube containing serum and the samples were then vortexed for 2 minutes and then centrifuged for 10 minutes at 11,000 rpm at room temperature using an Eppendorf centrifuge.

The supernatant was transferred from the Eppendorf tube and placed into a clean labelled 1.7 mL polypropylene microtube and evaporated for dryness (three hours) in a Genevac EZ-2 Plus at 35° C. on the medium boiling point setting. The samples were placed in the freezer (−20° C.) overnight. The following morning, the samples were allowed to come to room temperature before proceeding with the next step.

The residues were re-suspended in 80 μL of solution 2 and vortexed for 2 minutes then centrifuged for 2 minutes at 11,000 rpm at room temperature using Eppendorf centrifuge.

The solution was carefully transferred to HPLC vials with inserts and capped with HPLC caps and 25 μL was injected to the LCMS for analysis.

HPLC-UV Instrumentation and Conditions

The following methods were used for the LCMS analysis:

Isocratic chromatographic separation was performed on a C18 column (Zorbax eclipse XDB C18 column, 4.6×150 nm, 5 micron particle size Agilent USKH009316) with guard using a mobile phase of methanol (0.2% formic acid): water (0.4% formic acid), 50:50, at a flow rate of 0.75 mL/min for 6 minutes. The first two minutes was sent to the waste and berberine elutes between 3-4 minutes and chelerythrine elutes between 4.5-5.5 minutes. Their post time was 0.1 min. The column temperature was 40° C. and the autosampler temperature was maintained at 4° C. the sample injection volume was 25 μL and the injector is set to −10 mm with bottom sensing enabled. A 4000 Q trap from AB Sciex Instruments equipped with an electrospray ionization (ESI) was used in the positive ion mode with multiple reaction monitoring (MRM) for the quantitative analysis. Nitrogen was used as the collision gas and the curtain gas. The curtain gas was 10.00 psi, the collision gas was 10, and the ion spray voltage was 5500 volts, the temperature was 600° C., and gas sources 1 and 2 were 30 psi. The declustering potential was 110 volts, the exit potential was 10.00 volts, the focusing lens 1 was −10.50 volts and the cell exit potential was 4.00 volts. Quantification was performed using the transitions m/z 336.08→292.1 (CE=45 V, 100 msec) for berberine and 348.4→304.4 (CE=45 V, 100 msec) for chelerythrine chloride with low resolution. Analytical data was acquired and quantification processing was performed by using Analyst software.

### Example 16: Quantification of Berberine in Exemplary Formulation 9 from Example 15

Preparation of Formulations

The berberine hydrochloride used in this study was purchased from Sigma-Aldrich and the same lot was used for oral administration and preparation of the transdermal formulas. The 5% (w/w) berberine in an exemplary formulation 9 of table 16 and 5% (w/w) berberine in PLO was prepared by MNK Recherches (Montreal, QC).

Preparation of Standard Series

2.0-5.0 mg of powdered berberine hydrochloride (Sigma Aldrich B3251) was weighed into a scintillation vial and the mass was recorded. The recorded mass was then used to calculate the volume of methanol/water (50:50) needed to generate a final concentration of 1 mg/mL and that amount was accurately added to the vial using a pipette. The solution was vortexed for 30 seconds or until the berberine was completely dissolved.

A 50 μg/mL solution of berberine hydrochloride was prepared by addition 50 μL of 1 mg/mL stock solution to 950 μL of methanol/water (50:50) and the resulting solution was vortexed for 10 seconds. A 1 μg/mL solution of berberine hydrochloride was prepared by adding 20 μL of 50 μg/mL solution to 980 μL of methanol/water (50:50) and the resulting solution was vortexed for 10 seconds.

A 1 mg/mL solution of chelerythrine chloride (internal standard—Sigma Aldrich C2932) was prepared in 50:50 methanol/water. A 20 μg/mL solution of chelerythrine chloride was prepared by adding 20 μL of the 1 mg/mL solution of chelerythrine chloride to 980 μL 50:50 methanol/water and the resulting solution was vortexed for 10 seconds. A 1 g/mL solution of chelerythrine chloride was prepared by adding 50 μL of 20 μg/mL solution of chelerythrine chloride to 950 μL 50:50 methanol/water and the resulting solution was vortexed for 10 seconds.

A 1000 ng/mL solution of berberine hydrochloride+125 ng/mL solution of chelerythrine chloride was prepared by adding 40 μL of 50 μg/mL solution of berberine hydrochloride and 5 μL of 50 μg/mL solution of chelerythrine chloride to 1.955 mL of methanol/water (50:50) (Solution 1) and the resulting solution was vortexed for 10 seconds. A 125 ng/mL solution of chelerythrine chloride was prepared by adding 5 μL of 50 μg/mL solution of chelerythrine chloride to 1.995 mL of methanol/water (50:50) and the resulting solution was vortexed for 10 seconds (Solution 2). Solution 1 was serial diluted with solution 2 (100 μL) in HPLC vials with inserts to give a standard series with concentrations of 1000, 500, 250, 125, 62.5, 31.25, 15.62, 7.8, 3.9 ng/mL of berberine and a constant concentration of 125 ng/mL solution of chelerythrine.

Ratio of peak area of berberine to peak area of chelerythrine chloride was plotted against berberine concentrations and used to produce a standard curves in the form of y=A+Bx using weighted least squares linear regression.

Control Samples

Preparation of Standards

Doped Cream—500 ng/mL

5-10 mgs of blank exemplary formulation 9 cream was weighed into a scintillation vial in duplicate. The mass was recorded and used to calculate the amount of 50 μg/mL solution of berberine to be added (mass of base formulation cream in mg % 100 gives μL of 50 μg/mL stock solution to add). Then enough methanol/water (50:50) was added to make up to 1 mg/mL (mass of base cream in mg subtract mass of base cream in mg % 100). The resulting solution was subjected to sonication for 30 minutes at room temperature. One mL of this solution was then transferred to a microcentrifuge tube by pipette and was centrifuged at 11000 rpm for 10 minutes. 10 μL of this solution was then added to 90 μL of a 137.5 ng/mL solution of chelerythrine chloride in methanol/water (50:50) (made by using 5.5 μL of 50 μg/mL solution to 1.9945 methanol/water (50:50)) in a HPLC vial with insert and the solution was mixed by pipette.

Spiked Cream—1250 ng/mL

5-10 mgs of blank exemplary formulation 9 cream was weighed into a scintillation vial in duplicate. The mass was recorded and used to calculate the amount of methanol/water (50:50) to add to make a 1 mg/mL solution and that was added by pipette. The resulting solution was subjected to sonication for 30 minutes at room temperature. One mL of this solution was then transferred to a microcentrifuge tube by pipette and was centrifuged at 11000 rpm for 10 minutes. 10 μL of this solution was then added to 90 μL of a 137.5 ng/mL solution of berberine chloride and 137.5 ng/mL solution of chelerythrine chloride in methanol/water (50:50) (made by using 5.5 μL of each of 50 μg/mL solutions of berberine chloride and chelerythrine chloride to 1.989 methanol/water (50:50)) in a HPLC vial with insert and the solution was mixed by pipette.

Blank Cream

5-10 mgs of blank exemplary formulation 9 cream was weighed into a scintillation vial in duplicate. The mass was recorded and used to calculate the amount of methanol/water (50:50) to add to make a 1 mg/mL solution and that was added by pipette. The resulting solution was subjected to sonication for 30 minutes at room temperature. One mL of this solution was then transferred to a microcentrifuge tube by pipette and was centrifuged at 11000 rpm for 10 minutes. 10 μL of this solution was then added to 90 μL of a 137.5 ng/mL solution of chelerythrine chloride in methanol/water (50:50) (made by using 5.5 μL of a 50 μg/mL solutions of chelerythrine chloride to 1.9945 methanol/water (50:50)) in a HPLC vial with insert and the solution was mixed by pipette.

Preparation of Unknown Samples

Unknown Samples

5-10 mgs of cream to be analyzed was weighed into a scintillation vial in duplicate. Samples were taken from several positions in the cream bottle, top, side and bottom. The mass was recorded and used to calculate the amount of methanol/water (50:50) to add to make a 1 mg/mL solution and that was added by pipette. The resulting solution was subjected to sonication for 30 minutes at room temperature. One mL of this solution was then transferred to a microcentrifuge tube by pipette and was centrifuged at 11000 rpm for 10 minutes. 10 μL of this solution was then added to 90 μL of a 137.5 ng/mL solution of chelerythrine chloride in methanol/water (50:50) (made by using 5.5 μL of a 50 μg/mL solutions of chelerythrine chloride to 1.9945 methanol/water (50:50)) in a HPLC vial with insert and the solution was mixed by pipette. Further dilution was performed by adding 10 μL of this solution to 90 μL of 125 ng/mL solution of chelerythrine chloride (made by adding 5 μL of a 50 μg/mL solution of chelerythrine chloride to 1.995 methanol/water (50:50)) in an HPLC vial.

HPLC-UV Instrumentation and Conditions

The following methods were used for the LCMS analysis:

Isocratic chromatographic separation was performed on a C18 column (Zorbax eclipse XDB C18 column, 4.6×150 nm, 5 micron particle size Agilent USKH009316) with guard using a mobile phase of methanol (0.2% formic acid): water (0.4% formic acid), (50:50), at a flow rate of 0.75 mL/min for 6 minutes. The first two minutes was sent to the waste and berberine elutes between 3-4 minutes and chelerythrine elutes between 4.5-5.5 minutes. Their post time was 0.1 min. The column temperature was 40° C. and the autosampler temperature was maintained at 4° C. The sample injection volume was 25 μL and the injector is set to −10 mm with bottom sensing enabled. A 4000 Q trap from AB Sciex Instruments equipped with an electrospray ionization (ESI) was used in the positive ion mode with multiple reaction monitoring (MRM) for the quantitative analysis. Nitrogen was used as the collision gas and the curtain gas. The curtain gas was 10.00 psi, the collision gas was 10, and the ion spray voltage was 5500 volts, the temperature was 600° C., and gas sources 1 and 2 were 30 psi. The declustering potential was 110 volts, the exit potential was 10.00 volts, the focusing lens 1 was −10.50 volts and the cell exit potential was 4.00 volts. Quantification was performed using the transitions m/z 336.08→292.1 (CE=45 V, 100 msec) for berberine and 348.4→304.4 (CE=45 V, 100 msec) for chelerythrine chloride with low resolution. Analytical data was acquired and quantification processing was performed by using Analyst software.

### Example 17: Determination of PCSK9 in Berberine-Treated Rat Serum

Samples

14 Zucker fatty rats (InterVivo) were employed in seven groups (two in each group). A total of 2 animals were orally-administered simvastatin (6 mg/kg/dose; Group A), 2 rats were orally-administered berberine (180 mg/kg/dose; Group B), 2 rats were administered metformin (200 mg/kg/dose; Group C), 2 rats were orally-administered only the vehicle (Group D), 2 rats were transdermally-treated with exemplary formulation 9 (Example 15) (3.6 g/kg/dose; Group E), 2 rats were transdermally-treated with exemplary formulation 9 (3.6 g/kg/dose) and orally-administered simvastatin (6 mg/kg/dose; Group F), and 2 rats were transdermally-treated with exemplary formulation 9 (3.6 g/kg/dose) and orally-administered metformin (200 mg/kg/dose; Group G).

The serum samples obtained were dense and cloudy, and most samples were red in color. The serum samples were centrifuged at 13,500 rpm for 30 minutes at 4° C. Floating fat was observed in the centrifuge tube. By avoiding the fat, only supernatant was used in the ELISA to determine PCSK9 protein.

Preparation of Formulations

The presence of PCSK9 protein in serum samples was determined by sandwich enzyme-linked immunosorbent assay (ELISA). The ELISA Strips were obtained from Greiner bio-one. The blocking agent was 10% Skim Milk powder, obtained from BioShop, in PBS-Tween (PBS containing 0.05% Tween 20). The capture antibody was rabbit polyclonal antibody to PCSK9 (aa 1-692) protein obtained from Sino Biological Inc (Rb pAb to PCSK9). The human PCSK9 recombinant protein: (Human PCSK9/NARC1 protein [His-tag], 1-692 amino acids) was obtained from Sino Biological Inc. The PCSK9 mouse monoclonal IgG1 was obtained from Santa Cruz Biotechnology. The detection antibody was peroxidase conjugated affinity pure goat anti-mouse IgG, Fcy subclass 1 specific obtained from Jackson Immuno Research Inc. DPBS is Dulbecco's phosphate buffered saline, or Gibco 14200-075, obtained from Life Technologies). PBS-Tween (PBST) is PBST buffer obtained from Bio Basic Inc. Substrate: TMB one component HRP microwell substrate was obtained from Bethyl. Stop Solution was ELISA stop solution obtained from Bethyl.

ELISA Procedure

The capture antibody was diluted to 1 μg/mL using DPBS, and then 100 μL of this diluted solution was added to each well of the ELISA strips. The wells were sealed by adhesive film, and kept in wet box at 4° C. for about 12 hours. The wells were then flipped, and 200 μL of blocking agent was added to each well. The plate was shaken vigorously at room temperature (RT) (250 rpm) for 2 hours. The wells were washed 5 times with PBS-T. PCSK9 recombinant protein was diluted by using PBST to get 200 ng/mL, 150 ng/mL, 100 ng/mL, 75 ng/mL, 50 ng/mL, 25 ng/mL, and 10 ng/mL PCSK9. The serum supernatant was diluted by a factor of 25 with PBST, and then the sample was added in triplicate. In absence of Zucker fatty rat normal serum (as negative control), the DPBS coated sandwich ELISA (instead of coating RbPAb to PCSK9, DPBS was coated to ELISA wells) was used as negative control, and the absorbance value of negative control was subtracted from the RbPAb to PCSK9 coated sandwich ELISA absorbance value. The plate was incubated at RT for 90 minutes, then washed 5 times with PBS-T. 100 μL of 1 μg/mL of mouse monoclonal antibody PCSK9 was added to the well, and the sample was incubated at RT for 1 hour. Detection antibody was diluted by a factor of 5000 in PBST, and 100 μL was added to each well. The plate was incubated at RT for 1 hour, then washed 5 times by using PBS-T. 100 μL of substrate solution was then added to well, and incubated at RT for color development. 100 μL of stop solution was later added to each well, and absorbance at 450 nm wavelength was measured by using SpectraMaxM2 spectrophotometer. The concentration of serum sample was calculated from the standard curve equation. The calculation was done on the average absorbance value for the diluted sample (after subtracting the negative control), and these values were plotted in the standard curve equation. From this, the concentration of the log 10 value was obtained. The log 10 value was converted into integer value, and this value was multiplied by dilution factor (25).

### Example 18: Effect of Berberine, Metformin and Simvastatin on Body Mass and Lipid Biomarkers

Samples

All in vivo animal husbandry, treatment regiments, and sample collections were completed by InterVivo. The study was completed using obese male Zucker fa/fa rats (Charles River) at 10 weeks of age. Animals receiving topical ointment applications had an area, approximately 2-inch square, shaved on the middle of the back between the shoulder blades. Shaving was performed under anaesthesia one day prior to initiation of test article administration. The area was further shaven, as needed, over the course of the dosing period to ensure accurate drug application and absorption. For dosing procedures, animals were restrained and the transdermal test articles applied in a uniform layer over the entire shaved area. The shaved administration area was cleaned daily with paper towel before the subsequent transdermal dose was administered. Animals receiving test articles via oral gavage were restrained, and a ball-tipped gavage needle (18G) attached to a syringe containing the dosing solution was first inserted into the mouth, and then into the stomach. To determine the appropriate depth of insertion of the needle, the position corresponding to the last rib was measured prior to insertion. Test articles formulated for oral dosing were dissolved in vehicle 0.5% (w/v) methylcellulose and 0.2% (v/v) Tween 80 in physiological saline.

Four control samples were run: oral administration of simvastatin (6 mg/kg/dose; Group A), oral administration of berberine (180 mg/kg/dose; Group B), oral administration of metformin (200 mg/kg/dose; Group C), and oral administration of the vehicle (Group D) (Table 19). Transdermally-administered exemplary formulation 9 (Example 15) was also used (3.6 g/kg/dose), both alone (Group E), and in combination with either orally-administered simvastatin (6 mg/kg/dose; Group F), or with orally-administered metformin (200 mg/kg/dose; Group G).

Procedure

Daily administration took place between the hours of 08:00 to 10:00 and 16:00 to 18:00 from Days 0 to 27. On Day 28, the animals were dosed in the morning only, prior to euthanasia and tissue collection.

Animal health observations were made once daily from Day −34 to −1 and twice daily from Day 0 through study conclusion.

Animal body weights were performed once weekly from Day −34 to −2 and daily from Day −1 to 28. Rats were weighed, and the scales were operated and maintained, according to standard operating procedures.

Food and water consumption measurements were taken over a 24 hour period, once weekly, from Days 0 to 28.

Blood was collected via tail nick on Day-1 to determine unfasted blood glucose level for group allocation. Whole blood was collected via the saphenous vein or other appropriate route on Days 0, 7, 14, 21 and 28 immediately prior to test article administration. For each collection, a maximum of 400 μL of blood was collected into heparin vials and stored at 4° C. until sent for analysis (Antech Diagnostics) of cholesterol and triglycerides. Non-fasting blood glucose was measured at the same time points using a glucometer (Accu-Chek Aviva), and glycated hemoglobin A1C levels were measured with the A1C Now+ Analyzer (PTS Diagnostics).

On Day 28, subjects were anaesthetized with isoflurane, and whole blood was collected via cardiac puncture 2.5 hours (+/−5 minutes) following treatment administration on this day. Blood was transferred into 2 green top heparin vials (˜600 μL), and stored at 4° C. until sent for the clinical chemistry test (Antech Diagnostics). The clinical chemistry test measures included alkaline phosphatase (ALP), alanine transaminase (ALT), blood urea nitrogen (BUN), calcium, creatinine, glucose, phosphorus, total bilirubin, and total protein. The remainder of blood was transferred into serum separator tubes and centrifuged at 3,500 g for 10 minutes at 4° C. This provided three 500 μL aliquots of serum for each subject. Immediately following this collection, animals were sacrificed by decapitation. Whole liver samples were collected from each animal, weighed, and immediately snap frozen in liquid nitrogen. Serum and frozen tissue samples were stored at approximately −80° C. until shipped to the Sponsor for analysis.

### IV. Results and Discussion

(1) Chemical Modification of Berberine

As expected, initial formulations of berberine with the transdermal base formulations produced an intense yellow color, causing staining on clothing and other materials. Due to the unsuitable color of berberine for this application, congeners with reduced spectroscopic properties have been considered as alternatives. Berberine can be converted to derivatives with muted spectral qualities by reduction of the double bonds in the berberine skeleton resulting in decreased conjugation. Reduction of one double bond results in the production of DHB, and the reduction of two double bonds produced THB (Liu et al., 2014). Interestingly, DHB displayed increased bioavailability compared to berberine (Respiratory et al., 2008). It has been reported that DHB converts to berberine in circulation following oral administration in rats.

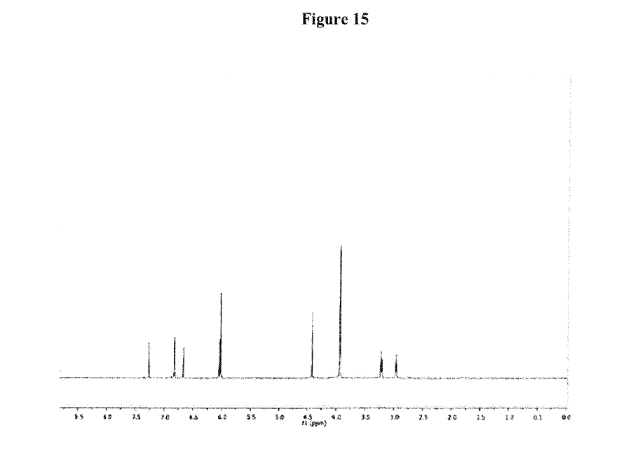

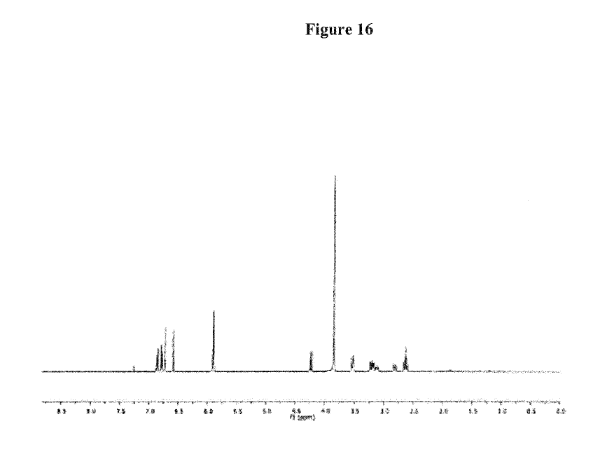

Berberine (87% purity, w/w) was obtained using its solubility in ethanol through the recrystallization of a commercial berberine extract (10% purity, w/w). This has been quantified using LC-UV instrumentation. Berberine can then be readily reduced to DHB and THB using readily available inexpensive reduction reagents. The reactions and purifications were performed under 8 hours producing the compounds in unoptimized yields of 50%. FIG. 15 illustrates the ¹H NMR spectrum of the isolated DHB having 90% purity. In separate experiments, it has been illustrated that DHB converts to berberine in solution and the impurities visible in the ¹HNMR spectrum of DHB is largely a result of this conversion. THB is ˜99% pure according to the ¹HNMR spectrum in FIG. 16. Elemental analysis indicated the presence of boron (potential contaminant from reagent) was below the limit of detection of both products (<0.3%).

(2) Stability of Synthetic Derivative, DHB

The stability of the synthetic derivatives was evaluated over time and although DHB was prone to oxidation to provide berberine after a short time, THB displayed excellent stability in several solvents.

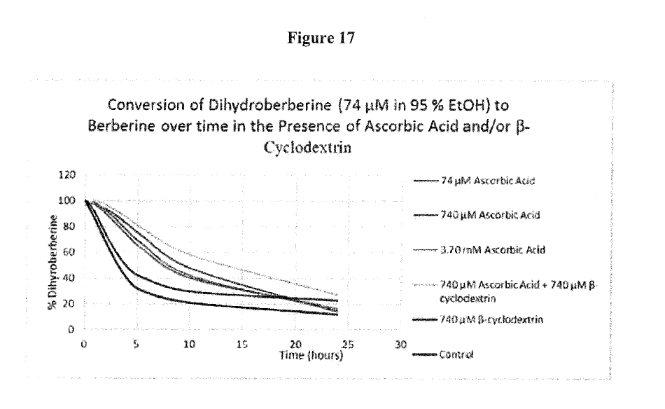

UV/VIS spectrometry is a useful tool for monitoring the conversion of DHB to berberine in solution. Several anti-oxidants and encapsulating agents were combined with DHB in solution to increase stability. Addition of ascorbic acid or cyclodextrin significantly slowed the oxidation of DHB to berberine. When both excipients were used, this effect was compounded as demonstrated in FIG. 17.

(3) In Vitro Testing of Berberine, DHB and THB

Proprotein convertase subtilisin/kexin type 9 (PCSK9) post-transcriptionally downregulates the low-density lipoprotein receptor (LDLR) by binding to the receptor's epidermal growth factor repeat A on the cell surface and shuttling the LDLR to the lysosomes for degradation. Mutations in the PCSK9 gene have been shown to cause either hypo- or hypercholesterolemia. Previous reports indicate that berberine has lipid lowering effects in both animal models and human trials (Arrigo F. G., Cicero, L., Rovati C. et al., 2007). The signalling effects of berberine, DHB, and THB were evaluated using the human liver cell line HEPG2. PCSK9, an enzyme that acts in cholesterol homeostasis, is expressed in HEPG2 cells and these cells have previously been used as a model of dyslipidemia. PCSK9 binds to LDL receptor causing uptake of LDL and the LDL receptor and targets both molecules for degradation upon internalization. In the absence of PCSK9, LDL and LDL receptor are still internalized, however, LDL receptor is not degraded but instead recycled to the cell surface resulting in more efficient uptake of circulating LDL. The effect is the reduction of circulating LDL levels and hence, reduction of cholesterol and triglyceride levels (Brown, M. S., 2006).

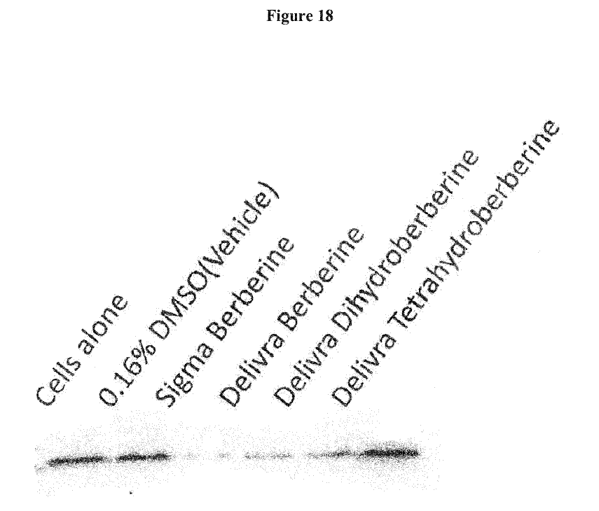

Testing of PCSK9 expression in supernatant from HEPG2 cells treated with berberine, DHB or THB confirmed that both berberine and DHB do indeed reduce the expression of PCSK9 down to a concentration of 6.25 μg/mL. THB did not appear to reduce expression of PCSK9 in comparison with the negative control, as illustrated on FIG. 18. This finding indicates that like berberine, DHB can be used for lowering PCSK9 and also would likely lower cholesterol levels.

(4) Transdermal Delivery of Berberine in Humans and Rodents

A transdermal delivery study was first carried out in humans using a topical formulation 3 comprising berberine. The formulation was applied to the forearm of a subject and blood samples were analyzed for berberine content. FIG. 14 shows a chromatogram of a serum blood sample demonstrating the presence of berberine within the circulation of the individual following application of the formulation of Table 4. As FIG. 14 illustrates, berberine was identified in the blood within 15 minutes of topical administration. In addition, berberine was only detected in the sera where the individual has received treatment transdermally with formulation 3. Taking the matrix effect into consideration, the concentration of berberine was determined to be 2.3 ng/mL.

This proof-of concept principle experiment demonstrated that transdermal formulations of commercially available berberine extracts within the exemplary formulations of the present application, are capable of introducing the bioactive berberine into systemic circulation.

Similarly, a transdermal delivery study was carried out in rodents wherein formulations 3, 3a and 4 of the present invention were compared in Sprague-Dawley rats. For each formulation two Sprague-Dawley rats were shaved and treated with 0.5 grams of transdermal product on the dorsal midline, posterior to shoulder blades. For each time point the concentration of berberine was determined in serum, as illustrated in Table 16.

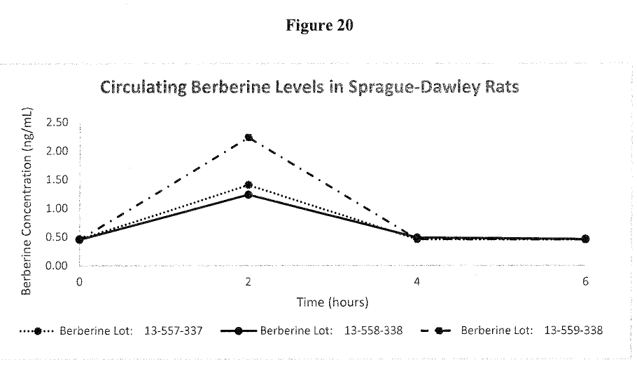

For each formulation, the levels of berberine were averaged (N=2) for each time point. Control concentrations of berberine were within acceptable ranges with a spiked berberine concentration of 97% (+/−5.5% S.E.M.) indicating negligible ionization matrix effects between standards and unknown samples (data not shown). Control concentrations of doped berberine demonstrated an extraction efficiency of 72% (+/−3.3% S.E.M.) indicating an acceptable and consistence extraction procedure (data not shown). With respect to berberine concentrations in rat serum, all treated animals demonstrated an increase two hours post-treatment with slight or negligible concentrations at four and six hours. A graphical representation of the tabulated data in Table 16 is shown in FIG. 20.

The formulations as disclosed herein have been shown to deliver berberine transdermally into the bloodstream in humans and in animals.

As described above in the introduction, berberine has been shown to be useful in the treatment of diabetes, particularly type II diabetes, hyperlipidemia, heart diseases, inflammatory diseases, skin disorders, metabolic disorders, neurological disease, infection resistance, and cancers including hepatoma, colon cancer, lung cancer, breast cancer and leukemia. In addition, berberine is thought to be useful as an anti-microbial agent for the treatment of disorders such as contact dermatitis, eczema and rosacea. The primary issues with the use of berberine as a therapeutic has been the poor bioavailability of the compound with only a small fraction of an oral dose entering the circulatory system. Furthermore, first-pass metabolism is suspected to quickly modify and excrete berberine.

The transdermal delivery of berberine using the formulations of the present application may result in increased total bioavailability of berberine and effectively avoids first-pass biotransformation of berberine. Thus, the formulations disclosed herein may be useful for the treatment of diseases and/or disorders that are responsive to berberine. It follows that the compositions disclosed herein may be used for the treatment of diabetes, particularly type II diabetes, hyperlipidemia, heart diseases, inflammatory diseases, skin disorders, metabolic disorders, neurological disease, infection resistance, and cancers including hepatoma, colon cancer, lung cancer, breast cancer and leukemia.

(5) Oral and Transdermal Bioavailability of Berberine in Rat Serum

The pharmacokinetics of berberine in an in vivo rat model were investigated by comparing the use of formulation 9 of the present application to a PLO formulation (commonly used vehicle for drugs) containing the same concentration of berberine, and an oral treatment group (Example 15). The purpose of this investigation was to provide a proof of concept for a proposed topical berberine containing product and aims to determine whether the exemplary formulation 9 of the present invention is superior to the PLO formulation in its ability to introduce berberine into systemic circulation.

The inabilities to identify a clear increase of berberine in serum with the oral treatment group is not surprising given the published pharmacokinetics for this route demonstrating a Tₘₐₓ at ˜15 minutes and almost complete clearance of the compound within 30 minutes of administration. Note, the timepoints included were chosen in order to investigate a transdermal application, which typically have slower penetrance and therefore longer Tₘₐₓ values. However it is hypothesized that formulation 9 would yield higher serum concentrations of berberine as peer-reviewed publications demonstrate a maximum observed concentration between 6 ng/mL to 20 ng/mL.

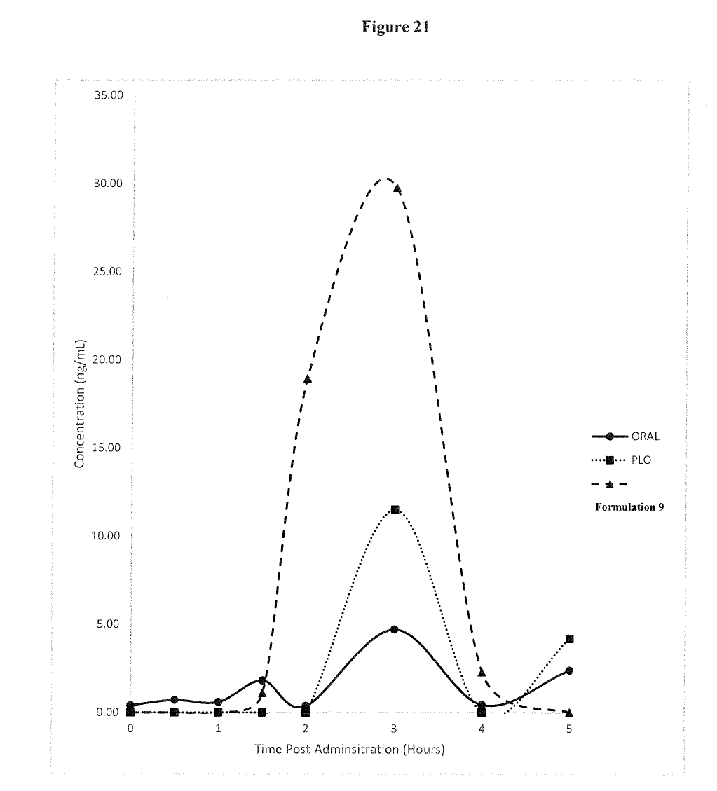

The average data (FIG. 21) suggests a strong distinction and superiority of formulation 9 and one-way ANOVA of all timepoints yields statistical significance (p<0.05). However, there is no distinct timepoint that yields statistical significance due to the high level of variation encountered in this study. However the level of accuracy and scrutiny employed in the quantification procedure suggests the variance is not introduced during sample processing or analysis.

This investigation successfully demonstrated that formulation 9 of the present application can introduce berberine into systemic circulation and yields an overall exposure level that is superior to PLO.

(6) Quantification of Berberine in Exemplary Formulation 9 Base Cream

A study was conducted to extract and quantify the berberine in exemplary formulation 9 and PLO cream formulations to establish the stability and consistency of berberine in creams used in the rodent study of Example 15. There was a high level of variability measured in the berberine rat serum concentrations as detailed above, and the aim was to confirm or deny the association between the variability observed in the blood samples and variability in the concentration of berberine in the cream formulations.

The standard series obtained was not linear over the concentrations tested. Trendlines were generated and used based on the partial standard series in the concentration range where the unknowns were observed. The limit of detection and limit of quantification was <1.9 ng/mL. Control concentrations of berberine were within acceptable ranged with spiked berberine concentrations of 91.3% for cream samples indicating matrix effects are negligible between standards and unknown samples (data not shown). Control concentrations of doped berberine demonstrated an extraction efficiency of 103.2% from cream samples indicating an acceptable extraction procedure (data not shown).

The berberine containing creams to be analyzed were those used in the study summarized in Example 15 with the goal of determining the stability and consistency of the analyte within and throughout the cream.

The exemplary formulation 9 cream bottle was sampled from three positions (top, bottom, side) in duplicate and the PLO cream was sampled at the top and bottom of the container in duplicate. The average concentration of exemplary formulation 9—berberine cream (Lot #VRI3-15082-CV), was 4.85% w/w +/−0.11 (mean+/−SEM) and 4.60% w/w +/−0.12 (mean+/−SEM) for the PLO-berberine cream (Lot #VRI3-15080-CV). Using the two-tailed P value equals 0.1693, by conventional criteria this difference is considered to be not statistically significant.

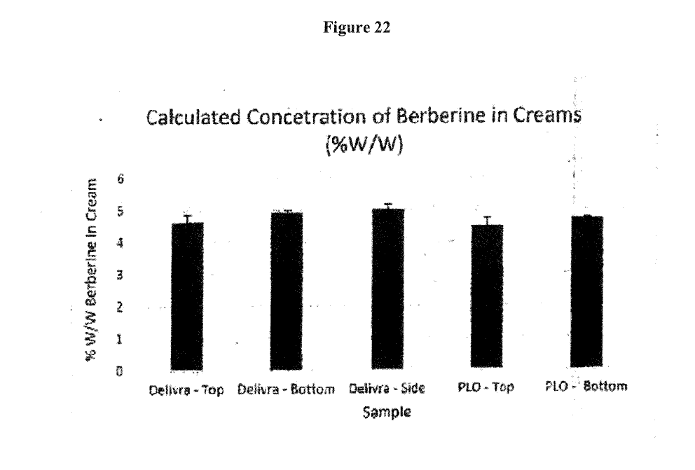

The three positions of the exemplary formulation 9 cream (top, bottom, side) sampled has calculated % w/w of 4.62% w/w +/−0.24 (mean+/−SEM), 4.92% w/w +/−0.06 (mean+/−SEM) and 5.01% w/w +/−0.16 (mean+/−SEM), respectively, as illustrated in FIG. 15. The PLO-berberine cream was sampled at the top and bottom of the bottle and had calculated concentrations of 4.49% w/w +/−0.25 (mean+/−SEM) and 4.71% w/w +/−0.03 (mean+/−SEM), respectively, as illustrated in FIG. 22. The differences between all of the creams/positions tested were determined to be not statistically significant.

This data suggests that the large variation in berberine serum concentration observed in Example 15 (Table 18, FIG. 21) is not the result of variation in the cream berberine concentrations. The data suggests that the cream retains its integrity of both in terms of stability and consistency over the time period studied.

(7) Determination of PCSK9 in Berberine-Treated Rat Serum

Proprotein convertase subtilisin kexin 9 (PCSK9), also named neural apoptosis-regulated convertase 1 (NARC-1), is a member of the proteinase K subfamily of subtilisin-related serine endoproteases. PCSK9 is produced predominately by the liver, secreted into plasma, and circulates at concentrations ranging from 100-1000 ng/mL (Konard et al., 2011). The full-length PCSK9 protein has 692 amino acids, including a signal peptide, a pro-domain, and a catalytic domain. It is initially synthesized as a soluble 74 kDa precursor protein. In the endoplasmic reticulum, it undergoes autocatalytic intramolecular cleavage to generate a 14 kDa pro-domain and a 60 kDa catalytic domain. These two domains remain associated when PCSK9 is secreted outside the cells (Seidah et al., 2003). The function of PCSK9 (as a secreted serine protease) is degradation of low density lipoprotein receptor (LDLR) on the surface of liver cells, which is directly correlated with its tight association with plasma cholesterol levels and a new therapeutic target to combat hypercholesterolemia coronary artery disease (Zhang et al., 2007). In addition, PCSK9 is an important regulator of plasma low-density lipoprotein (LDL)-cholesterol (LDL-C) concentrations (Lakoski et al., 2009).

Many lipid lowering oral drugs are now commercially available in the market, such as statins (simvastatin). Statins has been shown to enhance the expression of PCSK9 gene through SREBP pathway (Attie and Seidah, 2005), and also to cause an increase in the concentration of serum PCSK9 (Liu et al., 2013). Berberine, an isoquinoline plant alkaloid, has been demonstrated to lower fasting triglyceride levels in a clinical trial, and to reduce body weight as well as improve dyslipidemia in high fat diet-fed rats (Lee et al., 2006). Therefore, the effect of berberine (cream form as transdermally-administered exemplary formulation 9 (Example 15), and powder form as oral delivery) on plasma PCSK9 circulation concentration in rats, and the mechanism involved, was evaluated (Example 17). In the present investigation, an effort was made to determine the concentration of PCSK9 protein in the barberine treated rat serum by enzyme-linked immunosorbent assay (ELISA).

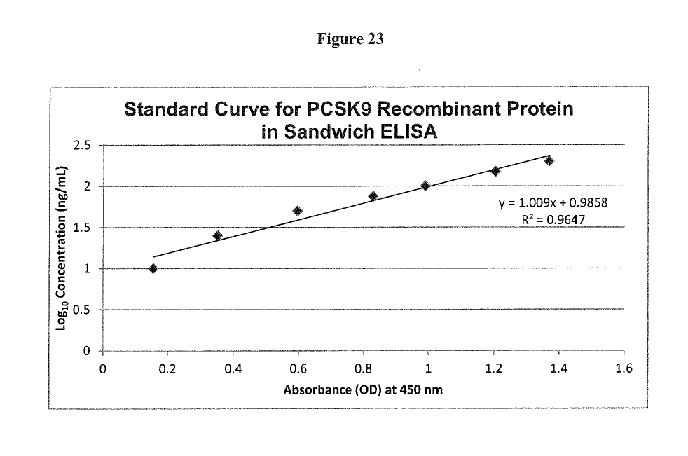

The standard curve for the PCSK9 recombinant protein (FIG. 23) was obtained by plotting the average of the duplicate samples. Based on the equation obtained in the standard curve, the PCSK9 protein concentration was determined (Table 20).

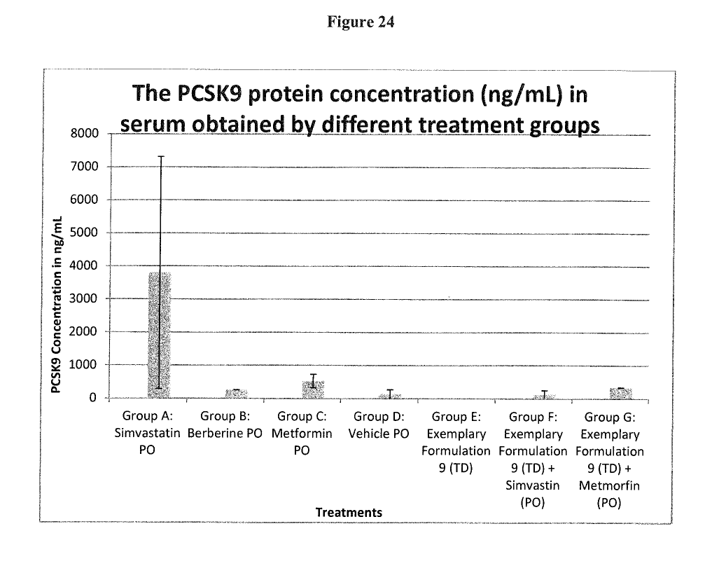

The PCSK9 concentration (Table 20, FIG. 24) was 3799 ng/mL (mean+/−2483 ng/mL) for Group A, 259 ng/mL (mean+/−2 ng/mL) for Group B, 525 ng/mL (mean+/−286 ng/mL) for Group C, and 132 ng/mL (mean+/−187 ng/mL) for Group D. Group E, transdermally-administered exemplary formulation 9, had a PCSK9 concentration of 0 ng/mL. The PSCK9 concentration of Group F was 124 ng/mL (mean+/−175 ng/mL), and Group G was 332 ng/mL (mean+/−7 ng/mL).

This data suggests that the PCSK9 concentration varied in some groups. However, this variation may be explained as sample preparation error or treatment error, or an insufficient sample size. The simvastatin treatment (Group A) showed higher amounts of PCSK9 in serum. In comparison to vehicle control (Group D), the oral berberine (Group B) and oral metformin (Group C) treatments also generated more PCSK9 protein in serum. The transdermally-administered exemplary formulation 9 group (Group E) showed a decrease in PCSK9 concentration as compared to the other groups, especially the simvastatin treatment (Group A).

Although these results may not be conclusive due to the small sample size, in comparison to vehicle control (Group D), the oral delivery of simvastatin (Group A), metformin (Group C), or berberine (Group B) in Zucker rats may increase the levels of circulating PCSK9 in serum. In contrast, in animals treated with transdermally-administered exemplary formulation 9 (Group E), the levels of PCSK9 were below the limits of detection. Furthermore, the combinatorial use of transdermally-administered exemplary formulation 9 and oral simvastatin (Group F) yielded lower PCSK9 levels as compared to statin alone

(8) Effect of Berberine, Metformin and Simvastatin on Body Mass and Lipid Biomarkers.

The purpose of this investigation was to examine the effect of a test article, berberine, delivered by two routes, on body weight and blood lipid biomarkers in the male Zucker rat, a model of type-2 diabetes and metabolic disorder (Example 18). The primary measures were effect of the test article on daily body weight, and weekly measures of blood biomarkers (non-fasting whole blood glucose, glycated hemoglobin A1C, cholesterol, triglycerides). The test article was compared to positive controls metformin and simvastatin.

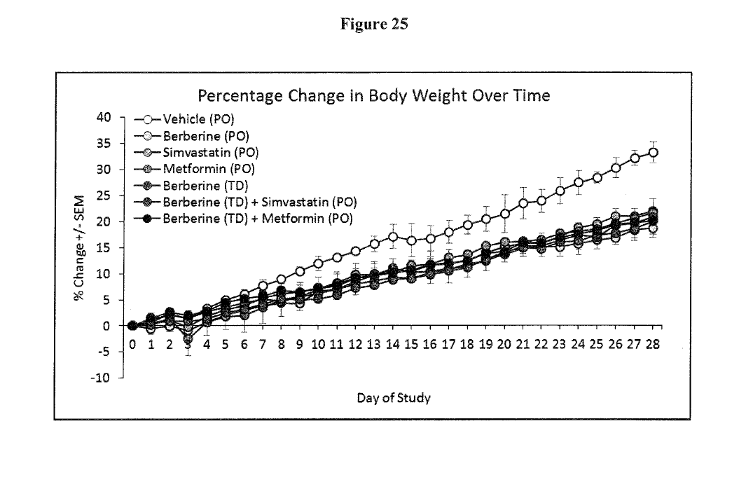

Percentage change in body weight was highest in the negative vehicle group compared to all other treatment conditions (FIG. 25; Tables 21-23). Berberine, either alone or in combination with simvastatin or metformin, was associated with reduced cholesterol and triglyceride blood counts. All treatments improved clinical chemistry measures associated with reduced liver and kidney function, as well as measures associated with low muscle mass or muscle wasting.

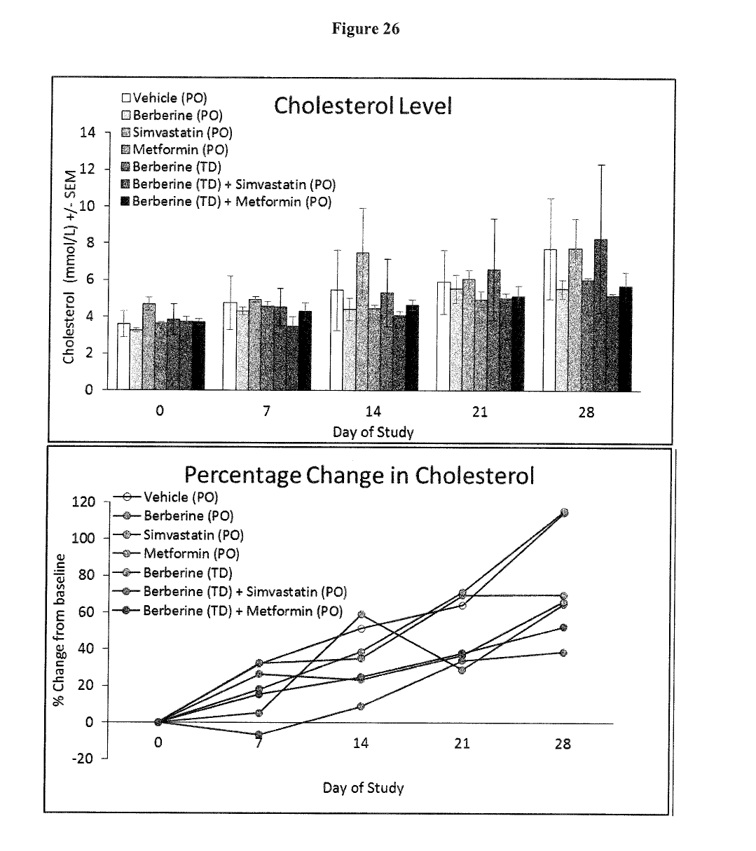

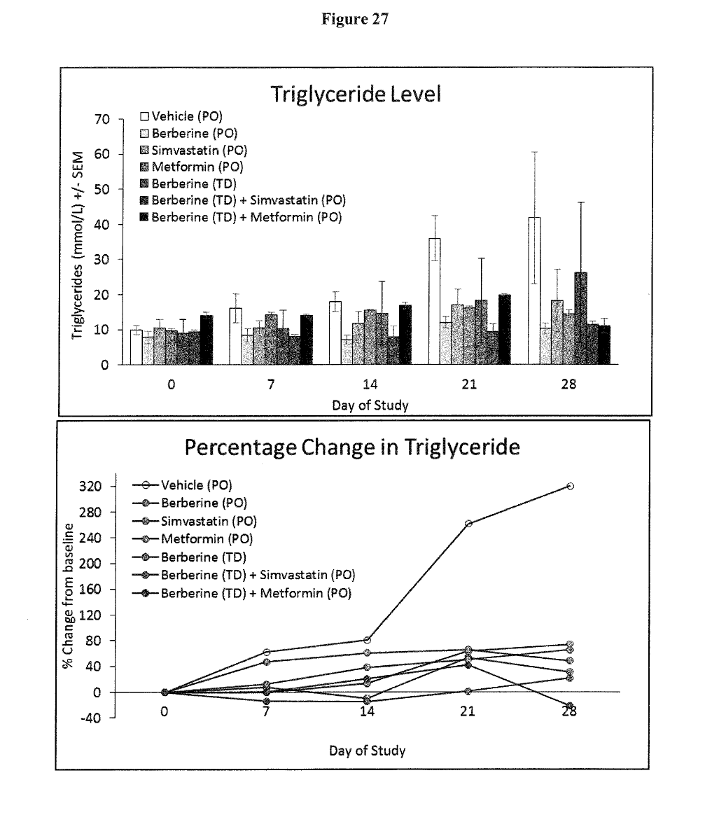

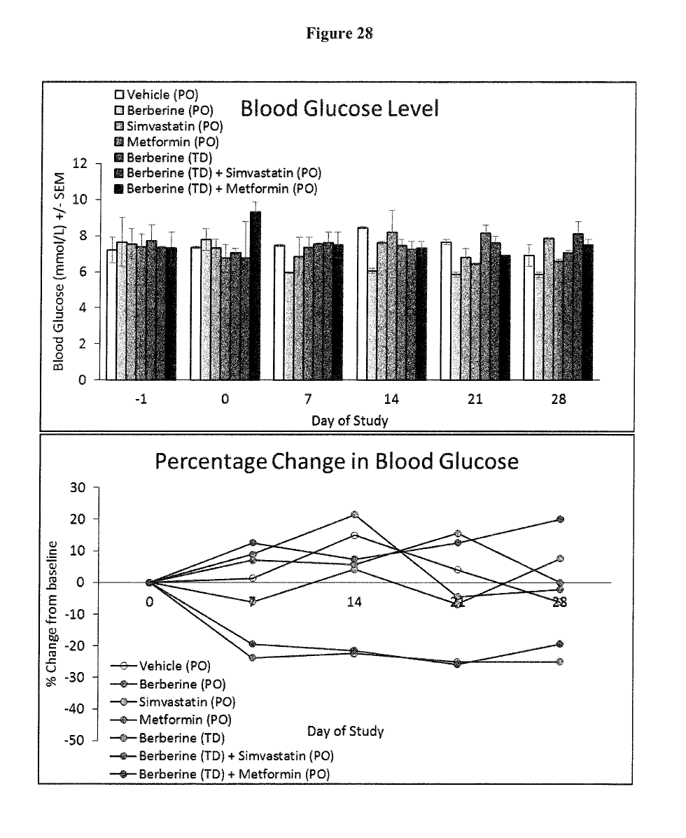

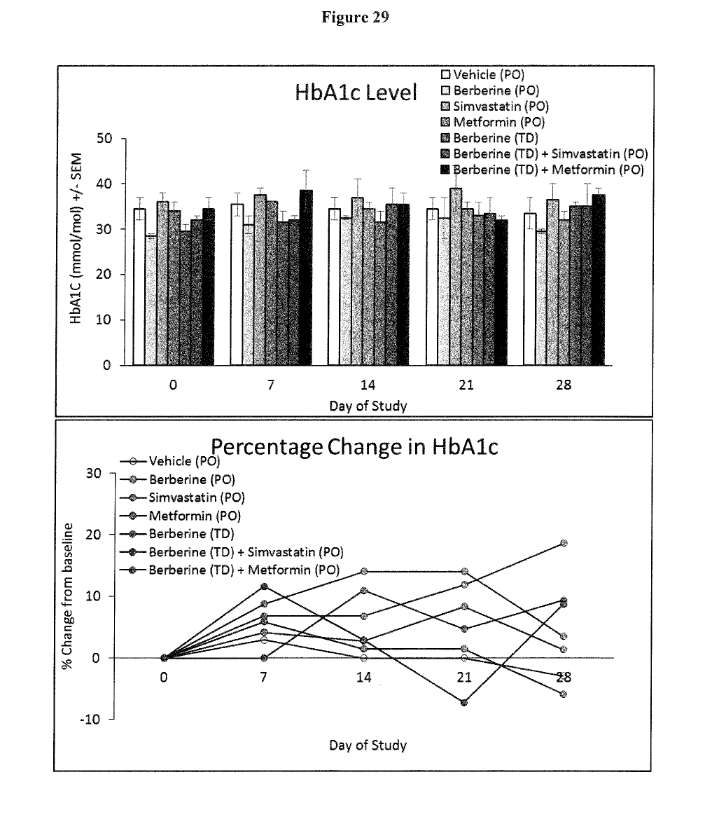

The placebo group demonstrated the largest percent baseline increase in triglycerides (FIG. 27; Table 29), and the second highest percent baseline increase in cholesterol (FIG. 26; Table 28). While all test articles showed association with reduction in the rate of triglyceride increase over time, transdermally-administered exemplary formulation 9 (Example 15) combined with metformin and simvastatin were associated with the lowest triglyceride levels. Similarly, the combination of transdermally-administered exemplary formulation 9 with metformin, and transdermally-administered exemplary formulation 9 with simvastatin, were associated with the lowest levels of cholesterol. Transdermally-administered exemplary formulation 9 alone was associated with the highest baseline increase in cholesterol. Oral berberine and the combination of transdermally-administered exemplary formulation 9 with metformin, but neither compound alone, were associated with the largest reduction in glucose levels (FIG. 28; Table 26), and metformin alone was associated with the largest reduction in HbA1c (FIG. 29; Table 27).

This investigation successfully demonstrated that berberine, alone or in combination with metformin or simvastatin, may reduce glucose and cholesterol levels.

(9) Effect of Berberine, Metformin and Simvastatin on Food and Water Intake.

The purpose of this study was to examine the effect of a test article, berberine, delivered by two routes, on food and water intake in the male Zucker rat, a model of type-2 diabetes and metabolic disorder (Example 18). The primary measures were effect of the test article once weekly food and water intake. The test article was compared to positive controls metformin and simvastatin.

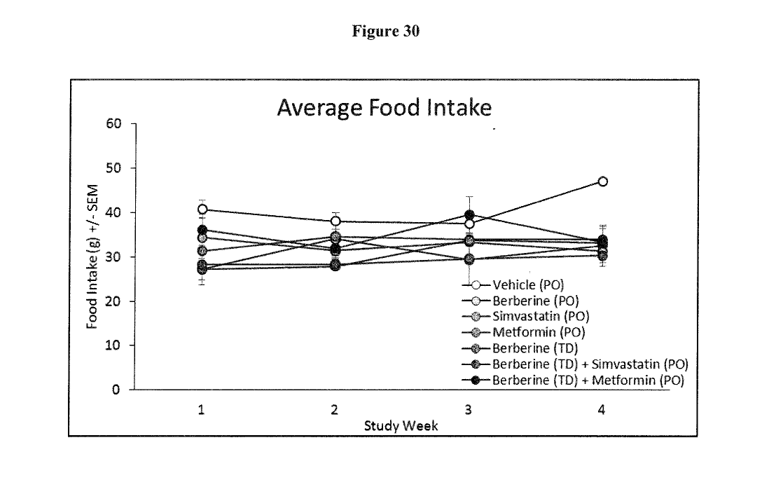

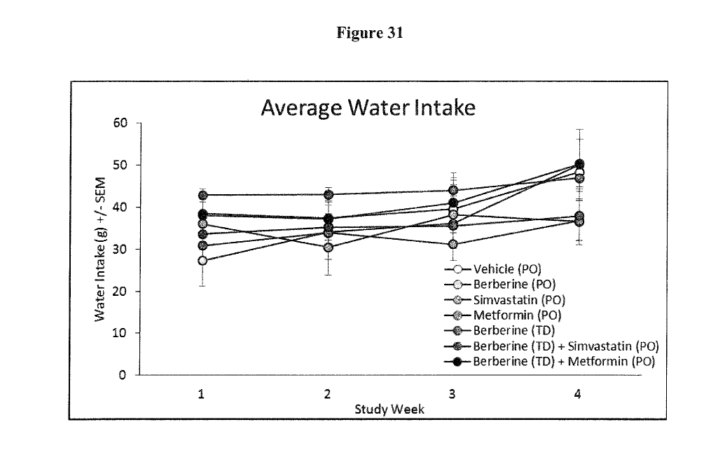

The placebo group was associated with the largest food intake at each time-point across the study, which was correlated to highest weight gain in the placebo group (FIG. 30; Tables 24-25). By contrast, transdermally-administered exemplary formulation 9 (Example 15), both alone and combined with simvastatin, was associated with the lowest food intake levels. Generally, water intake was most closely associated to the oral berberine and transdermally-administered exemplary formulation 9 groups, which may support a berberine-induced shift to increasing water consumption while consuming less food (FIG. 31). However, the association with increased water consumption while exposed to berberine may reflect increased nausea, which may be correlated to lower food consumption and higher water consumption. Combining transdermally-administered exemplary formulation 9 with simvastatin and metformin offered different results as compared to administration of simvastatin and metformin alone.

This investigation successfully demonstrated that transdermally-administered exemplary formulation 9 of the present application was associated with decreased food intake and increased water intake.

(10) Effect of Berberine, Metformin and Simvastatin on Clinical Chemistry Levels.

The purpose of this investigation was to examine the effect of a test article, berberine, delivered by two routes, on clinical chemistry levels in the male Zucker rat, a model of type-2 diabetes and metabolic disorder (Example 18).

High ALP and AST can be indicators of reduced liver function. The highest levels of ALP and AST were associated with the placebo group (vehicle only; Group D), suggesting liver function was most impacted in placebo controls (Table 30). With the exception of one animal in the transdermally-administered exemplary formulation 9 (Example 15) group, the highest bilirubin levels were associated with controls, which is consistent with lower levels of liver function. Similarly, the highest BUN and phosphorous levels were associated with the controls, which is suggestive of lower kidney function. The lowest protein and creatine levels were also associated with the control group, which can suggest reduced muscle mass or muscle wasting. Large liver weights were associated with the control group as well (Table 31).

Collectively, these results suggest that the treatments provided some level of rescue to the liver impairment expected in obesity of this nature.

While the present application has been described with reference to examples, it is to be understood that the scope of the claims should not be limited by the embodiments set forth in the examples, but should be given the broadest interpretation consistent with the description as a whole.

### Example 19: Efficacy of Transdermal Berberine Alone and in Combination with Pharmaceuticals in a Model of Metabolic Syndrome

Protocol Objectives:

This study examined the effect of a transdermal Berberine (formulation 9) compared with oral Berberine on body weight, food and water intake and blood lipid biomarkers in the male Zucker fa/fa rat, a model of type 2 diabetes and metabolic disorder. The primary measures are effect on daily body weight, once weekly food and water intake, and weekly measures of blood biomarkers (non-fasting whole blood glucose, hemoglobin A1C, cholesterol, triglycerides).

Study Design:

Fourteen obese male Zucker fa/fa rats at 5 weeks of age were housed for five weeks to the age of 10 weeks. During these five weeks, body weight measurements were taken once a week and once daily health observations were conducted for all animals.

One day prior to administration (T-1), non-fasting blood glucose was determined for each animal (theoretical range 150-350 mg/dL) and animals were randomized into groups based upon blood glucose level and body weight. The study design consisted of 7 groups (n=2/group) of Zucker rats at 10 weeks of age at commencement of the study. Animals received Berberine by oral gavage (PO) or transdermally (TD) twice a day, 7 days a week for 29 days (T0 to T28). Test articles formulated for oral dosing were dissolved in vehicle 0.5% (w/v) methylcellulose and 0.2% (v/v) Tween 80 in physiological saline or another similar method based on preliminary tests. Rats receiving transdermal berberine had an area shaved on the back between the shoulder blades which exposed the skin for application.

Immediately prior to initial treatment on Day 0 and on Days 7, 14, 21 and 28, blood was collected for measurement of triglycerides, cholesterol, Hemoglobin A1C, and non-fasting glucose. On Day 28, serum and whole liver was collected. Animal body weights were taken once weekly from Day −34 to −2, and daily from Day −1 to Day 28. Food and water intake measurements were taken once a week during the study period of Day 0 to Day 28. Measurements were taken 24 hours apart (+/−30 minutes) and the time of day was recorded with intake measures. There were seven groups of n=2 as shown in Table 32.

Test Article

A sufficient amount of test article was supplied and formulated. Test article product was administered at a dose of 3.6 g/kg twice a day (e.g. Simvastatin dosed at 6 mg/kg in AM and 6 mg/kg in PM). Stock solutions of Simvastatin and Metformin were made fresh twice a week. Berberine cream was weighed fresh daily at each AM and PM dosing to avoid desiccation of the test article.

Dose Frequency, Route and Duration of Administration:

Daily PO and TD dosing of the test articles took place between the hours of 08:00 to 10:00 and 16:00 to 18:00 for 7 days a week between Day 0 to 27. On Day 28, animals were dosed with test article between 08:00 to 10:00 only. The exact time of dosing (up to the minute) was recorded for each animal on the daily dosing sheets. The dose volume, 5 mL/kg for PO and 3.6 g/kg/dose for TD, was determined by each animal's body weight, as measured on the day of dosing. Remaining dosing solutions were stored at 4° C. between dosing periods.

Test Article Administration:

Animals which received topical transdermal application (according to Table 32) had an area of approximately 2″ square shaved on the middle of the back between the shoulder blades using clippers one day prior to test article administration. The animals shaved underwemt gaseous anaesthetic according to SOP ROD28.01 to ensure accuracy and avoid undue stress of the animal. The area was further shaven as needed over the course of the dosing period to ensure accurate drug application and absorption. At dosing, the animals were restrained and the transdermal test articles were applied in a uniform layer over the entire shaved area.

### I Test Article

| | | |
|---|---|---|
| i | Name | Berberine (oral) |
| ii | Dosage form | Oral Liquid |
| iii | Doses tested | 180 mg/kg PO (per os = oral) |
| iv | Manufacturing site | Delivra Inc. & MNK Recherche |
| v | Lot No. | Sigma-Aldrich (Cat. B3251; Lot. |
| | | SLBM9643V) from berberis asiatica |

### I Test Article

| | | |
|---|---|---|
| i | Name | 5% (w/w) transdermal-berberine |
| ii | Dosage form | Transdermal (TD) cream |
| iii | Doses tested | 3.6 g/kg TD |
| iv | Manufacturing site | MNK Recherche |
| v | Lot No. | Utilized berberine from Sigma-Aldrich |
| | | (Cat. B3251; Lot. SLBM9643V) from berberis |
| | | asiatica to produce MNK Recherche |
| | | formulation lot 15-360-17 |

### II Positive Control 1

| | | |
|---|---|---|
| i | Name | Simvastatin |
| ii | Dosage form | Oral Liquid |
| iii | Doses tested | 6 mg/kg BID |
| iv | Manufacturing site | Delivra Inc. |
| v | Lot No. | SA0150514 |

### III Positive Control 2

| | | |
|---|---|---|
| i | Name | Metformin |
| ii | Dosage form | Oral Liquid |
| iii | Doses tested | 200 mg/kg BID |
| iv | Manufacturing site | Delivra Inc. |
| v | Lot No. | J24Z046 |

Animals receiving test articles via oral gavage will be restrained and a ball-tipped gavage needle (18G) attached to a syringe containing the dosing solution will be inserted into its mouth and then into the stomach. To determine the appropriate depth of insertion of the needle, the position corresponding to the last rib will be measured prior to insertion of the needle.

### Blood Collection and Glucose Determination:

Whole blood was collected into green top heparin vials via the saphenous vein or another appropriate route on Days 0, 7, 14, 21, and 28 immediately prior to the test article treatment, and the exact time (up to the minute) of blood collection will be recorded for each animal. A maximum of 400 μL of whole blood was collected and stored at 4° C. until sent to Antech for analysis of cholesterol and triglycerides. Non-fasting blood glucose was measured using a glucometer (Accu-Chek Aviva, catalogue #0353231003) and hemoglobin A1C levels was measured with the A1C Now+Analyzer (PTS Diagnostics, product no. PTS3028).

### Health Observations and Moribundity.

General health observations were made once daily until 10 weeks of age then twice daily once test article administration had begun. Animals were observed for health abnormalities, decreased grooming, and signs of pain or distress. Animals underwent the euthanasia and tissue collection procedures if their body weight loss exceeded 20% of their peak body weight.

### Euthanasia and Tissue Collection

On Day 28, the animals were anaesthetized with isoflurane and whole blood was collected via cardiac puncture into red top blood collection tubes 2.5 hours (+30 min) following treatment. The animals were immediately sacrificed by decapitation and the time of euthanasia was noted to the minute for each animal. The whole liver was collected from each animal and immediately snap frozen in liquid nitrogen. Blood samples were centrifuged at 3,500×g for 10 minutes at 4° C., and 3×500 uL aliquots of serum was collected into 1.5 ml eppendorf tubes. Serum and frozen tissue samples was stored at −80° C. (+/−4° C.) until shipment on dry ice. Carcasses and unused tissues from the euthanized animals were disposed of according to standard operating procedures.

### Results and Discussion

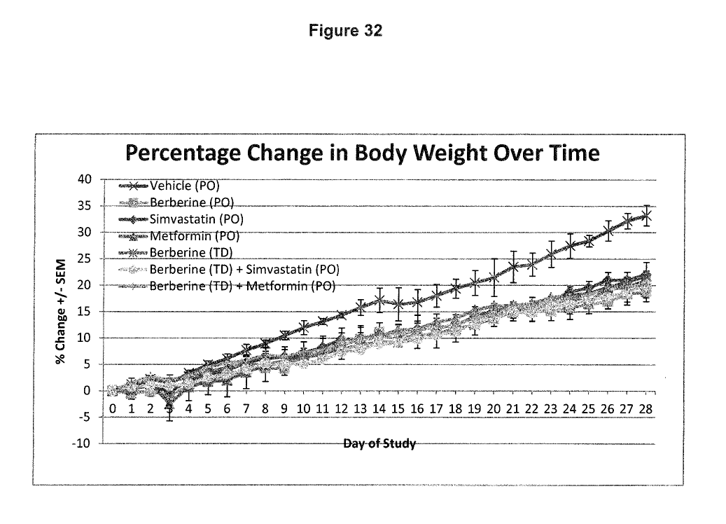

Overall, body animal body weights were measured once daily over the course of the experiment with control (Vehicle) animals yielding an average mass increase of 5.1 grams/day whereas all treatment groups yielded a statistically significant (as compared to vehicle) rate of ˜3.4 grams/day (see FIG. 32). The rate of weight gain (grams/day) for each group was 5.1, 3.3, 3.5, 3.3, 3.2, 3.2, and 3.3 for vehicle, oral berberine, simvastatin, metformin, transdermal-berberine, transdermal-berberine with simvastatin, and transdermal-berberine with metformin, respectively. All treatment groups demonstrated significant weight changes (P<0.01) as compared to vehicle.

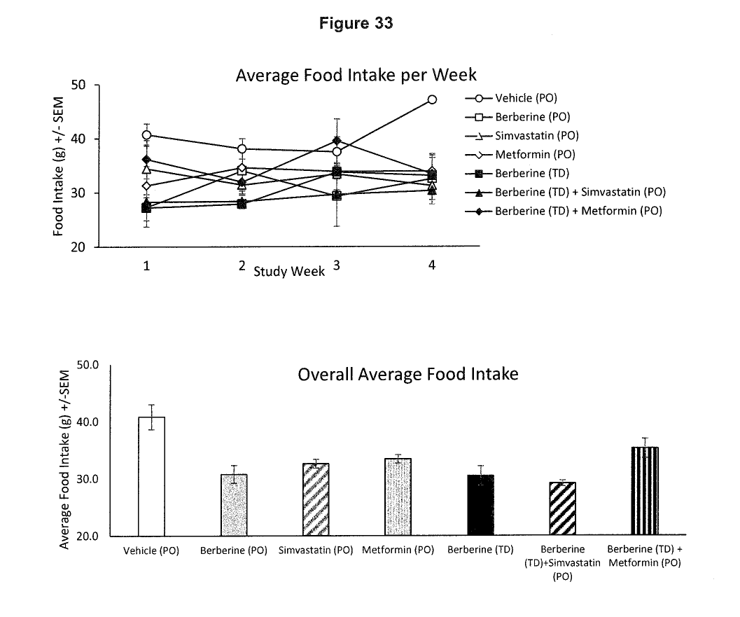

Food intake was measured for each week of the experiment and overall food consumption averaged over the entire experiment yielded a statistically significant decrease as compared to vehicle, except for animals treated with a combination of berberine (transdermal) and metformin (oral) (see FIG. 33). The overall average food intake (grams+/−SEM) for each group was 40.8+/−2.2, 30.8+/−1.5, 32.6+/−0.8, 33.4+/−0.7, 30.5+/−1.7, 29.2+/−0.5, and 35.3+/−1.7 for vehicle, oral berberine, simvastatin, metformin, transdermal-berberine, transdermal-berberine with simvastatin, and transdermal-berberine with metformin, respectively.

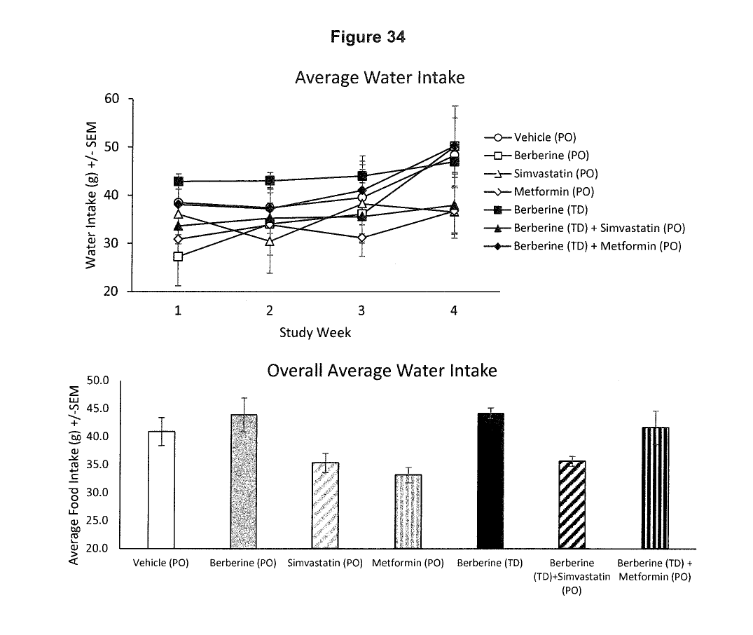

Water intake was measured for each week of the experiment and overall water consumption averaged over the entire experiment yielded a statistically significant decrease as compared to vehicle, except for animals treated with a combination of transdermal-berberine (transdermal) and metformin (oral) (FIG. 34).

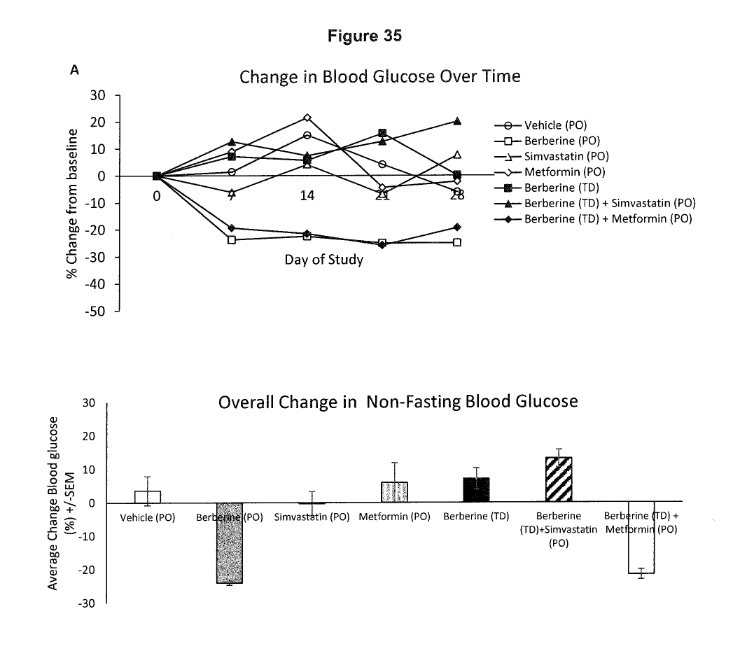

Non-fasting glucose levels were measured for each week of the experiment and overall glucose levels averaged over the entire experiment yielded a statistically significant decrease—as compared to vehicle—for those animals receiving oral berberine or the combination of transdermal-berberine with oral metformin (FIG. 35). The overall average glucose change (%+/−SEM) for each group was 3.5+/−4.3, −24.0+/−0.6, −0.3+/−3.7, 5.9+/−5.9, 7.1+/−3.2, 13.1+/−2.6, and −21.5+/−1.5 for vehicle, oral berberine, simvastatin, metformin, transdermal-berberine, transdermal-berberine with simvastatin, and transdermal—berberine with metformin, respectively. Oral berberine (P=0.001) and the combination of transdermal-berberine with metformin (P=0.002) demonstrated a statistical decrease in non-fasting glucose levels (asterisk).

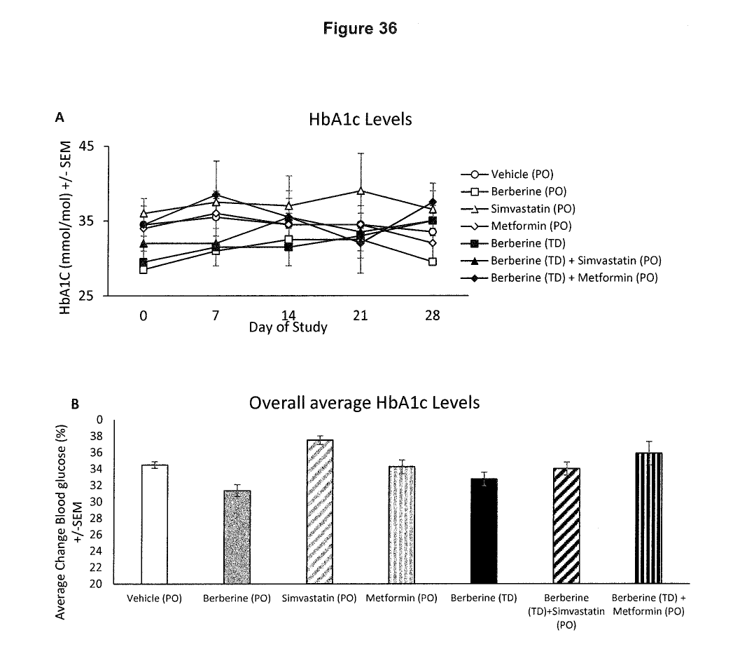

HbA1c is a measure of non-enzymatic hemoglobin glycation, a hallmark of increased and poorly controlled blood glucose levels. HbA1c is expressed as a ratio of glycated hemoglobin to normal hemoglobin. Levels were measured for each week of the experiment and overall HbA1c levels averaged over the entire experiment (see FIG. 36). The treatments indicating statistical significance across the study duration as compared to vehicle control were oral berberine and oral simvastatin, which demonstrated a decrease and increase in HbA1c, respectively. Glycated to non-glycated hemoglobin levels (Hba1C) levels were recorded weekly for each group (A) and overall change in glucose was determined for the duration of the experiment (B). The overall HbA1c level change (mmol/mmol+/−SEM) for each group was 34.5+/−0.4, 31.3+/−0.7, 37.5+/−0.5, 34.3+/−0.4, 32.8+/−0.8, 34.0+/−0.8, and 35.9+/−1.4 for vehicle, oral berberine, simvastatin, metformin, transdermal-berberine, transdermal-berberine with simvastatin, and transdermal-berberine with metformin, respectively. Oral berberine (P=0.001) treatment resulted in a decrease whereas oral simvastatin cause an increase.

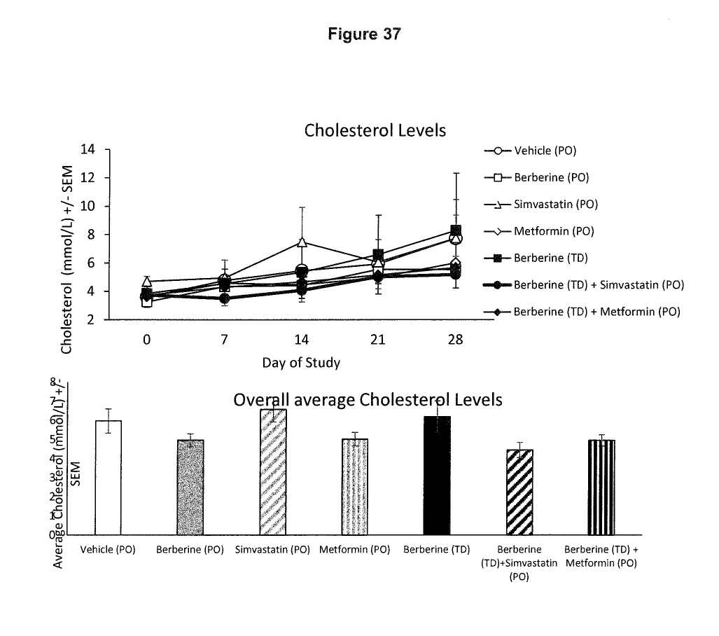

Cholesterol levels were measured for each week of the experiment and overall cholesterol levels averaged over the entire experiment (FIG. 37). No specific treatment group yielded an overall change (increase or decrease) in blood cholesterol, however the combinatorial effects of transdermal-berberine and oral simvastatin trend towards a decrease. Cholesterol levels were recorded weekly for each group (A) and overall average in cholesterol was determined for the duration of the experiment (B). The overall cholesterol level change (mmol/L+/−SEM) for each group was 6.0+/−0.8, 5.0+/−0.3, 6.6+/−0.6, 5.0+/−0.4, 6.2+/−0.8, 4.4+/−0.4, and 4.9+/−0.3 for vehicle, oral berberine, simvastatin, metformin, transdermal-berberine, transdermal-berberine with simvastatin, and transdermal-berberine with metformin, respectively.

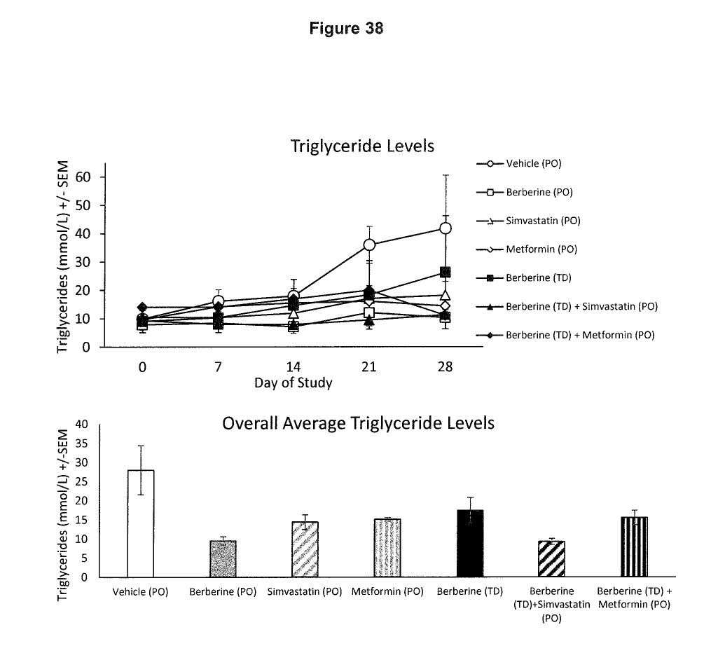

Triglyceride levels were measured for each week of the experiment and overall triglyceride levels averaged for the entire experiment (FIG. 38). All treatments resulted in a general decrease in triglyceride levels, however only oral berberine and the combinatorial treatment of transdermal-berberine with oral simvastatin yielded statistical significance. In addition, the combination of transdermal-berberine and oral simvastatin was statistically lower as compared to either therapeutic alone (FIG. 38). The overall triglyceride levels (mmol/L+/−SEM) for each group was 27.6+/−6.4, 9.5+/−1.0, 14.4+/−1.9, 15.1+/−0.4, 17.4 +/−3.3, 9.2+/−0.8, and 15.5+/−1.9 for vehicle, oral berberine, simvastatin, metformin, transdermal-berberine, transdermal-berberine with simvastatin, and transdermal-berberine with metformin, respectively.

Significant changes related to diabetic (blood glucose) and hyperlipidemic (triglyceride) biomarkers were observed when used in combination with the known prescription drugs metformin and simvastatin.

All treatment groups demonstrated a significantly lower rate of weight gain as compared to vehicle control (FIG. 32). This result can be expected from oral berberine with its known gastrointestinal upset and anti-microbial activity upon the gut microflora. Indeed, oral berberine treated animals demonstrated a significant reduction in food consumption (FIG. 32). However, a similar significant decrease was observed in weight gain and food consumption for transdermal-berberine (FIGS. 32 and 33) that suggests appetite suppression may be related to an additional mechanism. It is suggested that transdermal-berberine yields a decrease in weight gain and appetite suppression in tandem to the avoidance of well-known gastro-intestinal adverse events common to oral berberine consumption. With respect to non-fasting blood glucose levels (FIG. 34) only two treatments yielded a significant change in this biomarker, oral berberine and the combination of transdermal-berberine with metformin. Interestingly, metformin alone had no effect and the observation that this drug in combination with transdermal transdermal-berberine suggests these two treatments function in tandem to yield a decrease in non-fasting blood glucose. In regards to the diabetic biomarker HbA1c (FIG. 35) and the metabolic biomarker cholesterol (FIG. 36), only oral berberine yielded a resulting decrease in both parameters whereas no other treatment (alone or combinatorial) affected a change. In regards to blood triglyceride levels (FIG. 37) all treatments resulted in a general decrease in triglycerides, however only oral berberine and the combinatorial treatment of transdermal-berberine with oral simvastatin yielded statistical significance. In addition, the combination of transdermal-berberine and oral simvastatin was statistically lower as compared to either therapeutic alone, which indicates a combination therapy is more efficacious than either treatment alone.

Overall, transdermal berberine replicated the reduced weight gain and reduced food consumption observed for oral berberine. Moreover, the effects of transdermal-berberine were more pronounced when used in combination with diabetic drugs (metformin) for glucose regulation and lipidemic drugs (simvastatin) for triglyceride regulation.

### Example 20: Oral and Transdermal Berberine Bioavailability in Rat Serum Using Exemplary Formulation 9 and PLO

Rat serum from Example 15 was analyzed for the bioavailability of berberine. The linear range of quantification for this methodology was 0.4 ng/mL (10 pg on-column) to 200 ng/mL (5 ng on-column) with a limit of detection of 0.1 ng/mL and limit of quantification of 0.4 ng/mL. Extraction efficiencies within this range were 106%, 93%, and 93% with inter-assay coefficients of variance of 15%, 15%, and 10% at 2.5 ng/mL, 25 ng/mL, and 250 ng/mL respectively. The results of the berberine, berberine glucuronide, simvastatin and simvastatin hydroxy acid quantification were quantified.

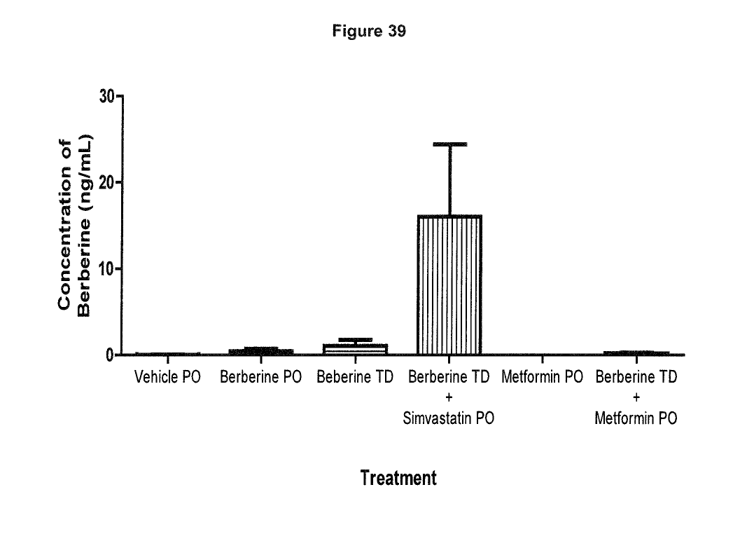

As shown in FIG. 39, plasma concentrations of berberine hydrochloride in rats (n=2) treated; orally [PO] with Vehicle (0.5% w/v methyl cellulose, 0.2% v/v Twee-80), orally with Berberine (180 mg/kg/dose), transdermally [TD] with Beberine (3.6 g/kg/dose), transdermally with Berberine (3.6 g/kg/dose)+orally with Simvastatin (6 mg/kg/dose), orally with Metformin (200 mg/kg/dose), transdermally with Berberine (3.6 g/kg/dose)+orally with Metformin (200 mg/kg/dose), were measured. The determined average concentration of berberine hydrochloride is illustrated. One-way ANOVA analysis demonstrates a significant difference across all treatments when compared to that of orally administered berberine.

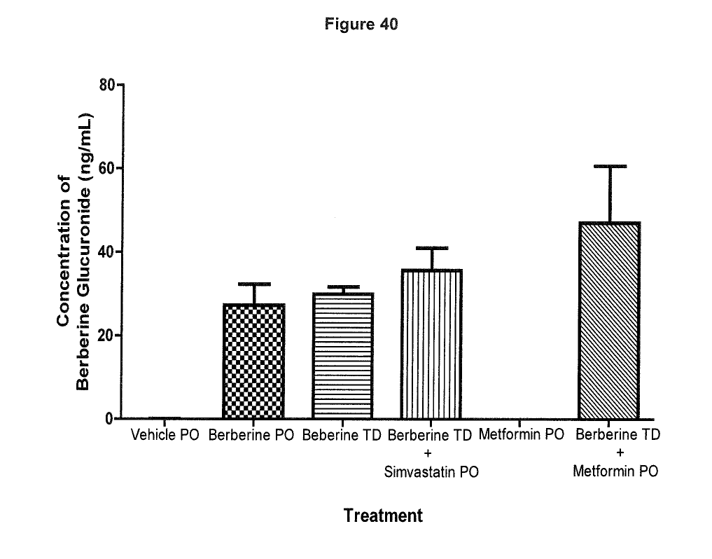

As shown in FIG. 40, plasma concentrations of berberine hydrochloride glucuronide in rats (n=2) treated; orally [PO] with Vehicle (0.5% w/v methyl cellulose, 0.2% v/v Twee-80), orally with Berberine (180 mg/kg/dose), transdermally [TD] with Beberine (3.6 g/kg/dose), transdermally with Berberine (3.6 g/kg/dose)+orally with Simvastatin (6 mg/kg/dose), orally with Metformin (200 mg/kg/dose), transdermally with Berberine (3.6 g/kg/dose)+orally with Metformin (200 mg/kg/dose), were measured. The determined average concentration of berberine hydrochloride glucuronide is illustrated. One-way ANOVA analysis demonstrates a significant difference across all treatments when compared to that of orally administered berberine.

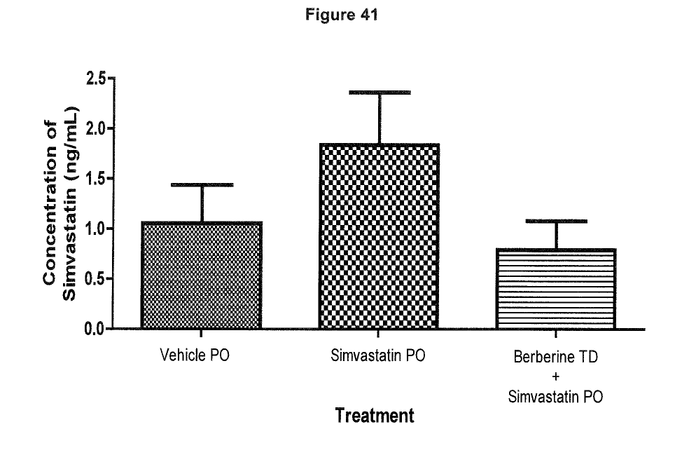

As shown in FIG. 41, plasma concentrations of simvastatin in rats (n=2) treated; orally [PO] with Vehicle (0.5% w/v methyl cellulose, 0.2% v/v Twee-80), orally with Simvastatin (6 mg/kg/dose), transdermally with Berberine (3.6 g/kg/dose)+orally with Simvastatin (6 mg/kg/dose), were measured. The determined average concentration of simvastatin is illustrated. One-way ANOVA analysis demonstrates a significant difference across all treatments when compared to that of orally administered simvastatin.

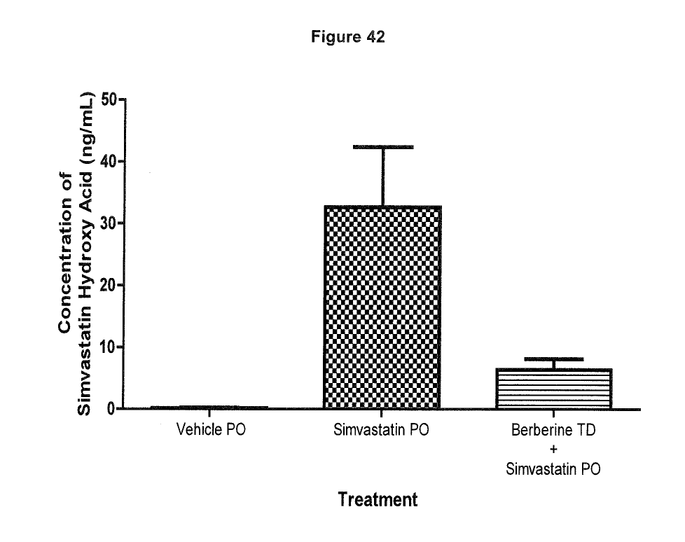

As shown in FIG. 42, plasma concentrations of Simvastatin hydroxy acid in rats (n=2) treated; orally [PO] with Vehicle (0.5% w/v methyl cellulose, 0.2% v/v Twee-80), orally with Simvastatin (6 mg/kg/dose), transdermally with Berberine (3.6 g/kg/dose)+orally with Simvastatin (6 mg/kg/dose), were measured. The determined average concentration of simvastatin hydroxy acid is illustrated. One-way ANOVA analysis demonstrates a significant difference across all treatments when compared to that of orally administered simvastatin.

### Discussion

The pharmacokinetic study quantified berberine, berberine glucuronide, simvastatin and simvastatin hydroxy acid in serum with a linear range that encompasses the concentrations observed in animals following administration of this compound via various routes of administration. The objective of this study was to examine the effect of two routes (oral and transdermal) on the delivery of berberine into systemic circulation and the influence of oral simvastatin on oral and transdermal delivery of berberine into systemic circulation. Oral administration of berberine resulted in low berberine plasma concentration of 0.44 ng/mL. Transdermally administered berberine using formulations of the disclosure increases berberine plasma concentration (1.07 ng/mL) compared to 0.44 mg of berberine observed in orally administered berberine. Orally administered simvastatin increased the plasma concentration of transdermally delivered berberine from 1.07 ng/mL to 16.00 ng/mL. This suggests that oral simvastatin may have effect on pharmacokinetic profile of transdermal berberine. Based on our findings, it is suggested that increased transdermal berberine concentration after oral simvastatin administration is possibly due to the competitive inhibition of CYP3A4 and P-gp by simvastatin, which is a dual inhibitor of both CYP3A4 and P-gp.

Oral Metformin decreased systemic berberine concentration (0.20 ng/mL) and also increased the metabolism of transdermally administered berberine which resulted in increased formation of berberine glucuronide metabolites (47.06 ng/mL). Oral administration of simvastatin resulted in 1.83 ng/mL systemic simvastatin concentration and 32.61 ng/mL simvastatin hydroxyl acid. Transdermal administration of berberine and oral administration of simvastatin resulted in low systemic concentrations of simvastatin (0.79 ng/mL) and 6.39 ng/mL simvastatin hydroxyl acid suggests that transdermal berberine decreases systemic circulation of simvastatin and simvastatin hydroxyl acid in oral simvastatin. This study has successfully demonstrated that transdermal administration can introduce berberine into the systemic circulation that is superior to oral route, and the increase in systemic berberine concentration by transdermal treatment can be further enhanced to about 16 fold by oral simvastatin.

### Example 21: Electron Micrographs of Transdermal Berberine and Dihydroberberine

Two formulations of DHB according to Example 26 and one formulation of BRB (formulation 9) were prepared as described below.

### Formulation a of Test Article 5% W/W DHB in Transdermal Formulation

Each lot was recorded using a work sheet. 0.5 g (+/−0.01 g) of dihydroberberine was weighed and placed into mortar. 8 g (+/−0.1 g) was weighed into a 20 mL syringe. The rest of the procedure was performed a steady flow of nitrogen at room temperature. The dihydroberberine (DHB) was ground by mortar and pestle for 10 minutes at which point it is a very fine powder/dust. 1.7625 mL of isopropyl myristate (IPM) was added to the mortar with the DHB and the resulting suspension was macerated with the pestle for 5 minutes. The weighed 8 g was then added to the mortar and the resulting suspension was macerated for 5 minutes with pestle. The formulation was then transferred to 2×20 mL syringes with a spatula. The syringes were equipped with a connector to another 20 mL syringe to prevent the formulation leaking out. The syringe was inverted and allowed any air to the top. The second syringe was disconnected and the air bubbles were then pushed out of the syringes. The second, empty 20 mL syringe was re-connected to the formulation containing syringe via connector. The formulation was pushed back and forth between the syringes 10× each way. The formulation was then stored at 4° C.

### Formulation B of Test Article 5% W/W DHB

0.5 g (+/−0.01 g) of dihydroberberine was weighed and placed into mortar. 8 g (+/−0.1 g) of transdermal formulation was weighed into two 20 mL syringes. The rest of the procedure was performed a steady flow of nitrogen at room temperature. The dihydroberberine (DHB) was ground by mortar and pestle for 10 minutes at which point it is a very fine powder/dust. 1.676 mL of isopropyl myristate (IPM) and the 0.088 mL of polysorbate 20 was added to the mortar with the DHB and the resulting suspension was macerated with the pestle for 5 minutes. The weighed 8 g was then added to the mortar and the resulting suspension was macerated for 5 minutes with pestle. The formulation was then transferred to 2×20 mL syringes with a spatula. The syringes were equipped with a connector to another 20 mL syringe to prevent the formulation leaking out. The syringe was inverted and allowed any air to the top. The second syringe was disconnected and the air bubbles were then pushed out of the syringes. The second, empty 20 mL syringe was re-connected to the formulation containing syringe via connector. The formulation was pushed back and forth between the syringes 10× each way. The formulation was then stored at 4° C.

Berberine formulations were prepared as in the earlier examples (Formulation 9).

For SEM, cream was placed in a tube cap and incubated in a sealed chamber with OsO4 for 4 hours vapour fixation. The fixed cream was placed directly on the imaging stub for imaging or washed with water and captured on a carbonate filter and then imaged. Images were collected using compositional backscatter mode under VP vacuum (BS) or secondary electron mode under variable pressure vacuum (UV).

### Results and Discussion

The images displayed patterns of liposomes however, the fixation in Osmium results in the material becoming impossible to dissociate, thereby suggesting that the material, although originally fluid, becomes completely crosslinked.

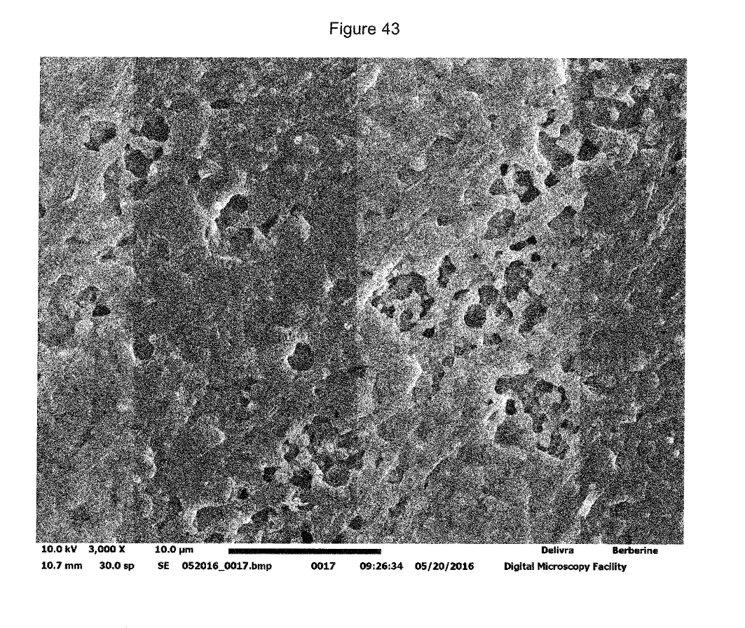

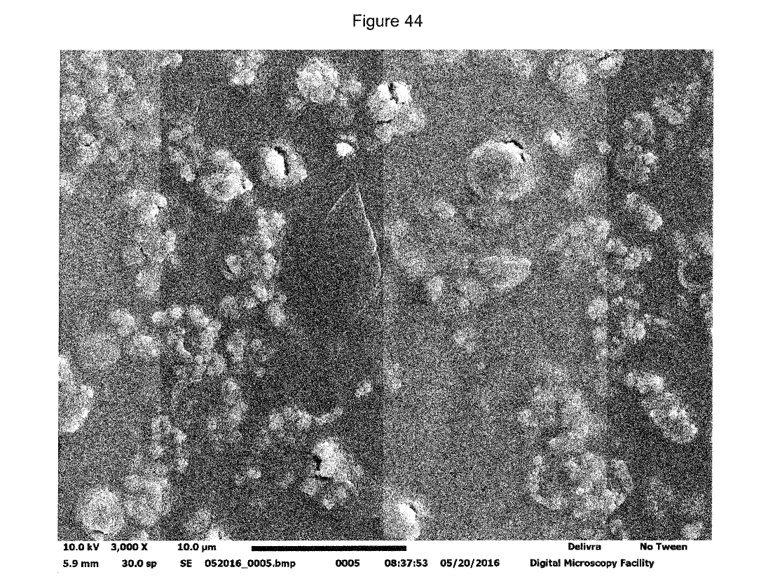

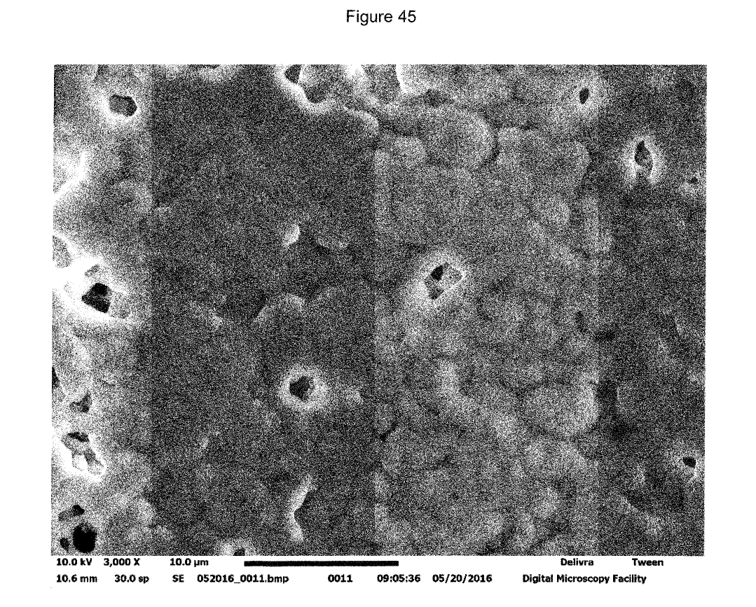

The formulations were spread in a thin layer on the surface of an aluminum SEM stub and imaged in variable pressure (VP) mode. The particles appeared to be whole liposomes or small groups of liposomes. FIG. 43-45 show the electron micrographs of the formulations.

### Example 22: Efficacy of Administration Routes and a Derivative of Berberine in a Model of Metabolic Syndrome

This study examined the efficacy of administration of a derivative of berberine (dihydroberberine) in transdermal formulations in 24 male Zucker rats (fa/fa), similar to the study protocol in Example 19. The treatment groups are shown in Table 33. The following formulations were prepared:

### Test Article 1

| | |
|---|---|
| Name | Berberine Transdermal (TD) - Formulation 9 |
| Dosage Form | Topical Cream, 5% (w/w) berberine |
| Doses Tested | 3.6 g/kg, BID |
| Lot # | 200416-01 |
| Manufacturer | Delivra Inc. |
| Drug storage during study | Refrigerated 2-4° C. |

### Test Article 2

| | |
|---|---|
| Name | Dihydroberberine Transdermal |
| | (TD) Transdermal-Example 26 |
| Dosage Form | Topical Cream, 5% (w/w) DHB |
| Doses Tested | 3.6 g/kg, BID |
| Lot # | 04152016/04082016 |
| Manufacturer | Delivra Inc. |
| Drug storage during study | Refrigerated 2-4° C. |

### Positive Control 1

| | |
|---|---|
| Name | Berberine Oral |
| Dosage Form | Powder in Liquid Vehicle |
| Vehicle | 0.5% (w/v) methylcellulose, 0.2% (v/v) |
| | Tween 80 in physiological saline |
| Dose Tested | 180 mg/kg, BID (5 ml/kg) |
| Lot # | BCBL6393V |
| Manufacturer | Sigma Aldrich |
| Drug storage during study | Refrigerated 2-4° C. |

### Negative Control 1

| | |
|---|---|
| Name | Transdermal Base |
| Dosage Form | Topical Cream |
| Dose Tested | 3.6 g/kg, BID |
| Lot # | 04152016P |
| Manufacturer | Delivra Inc. |
| Drug storage during study | Refrigerated 2-4° C. |

Results and Discussion

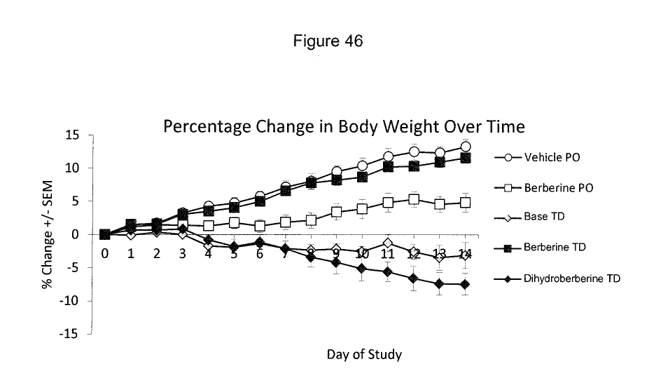

Overall, body animal body weights were measured once daily over the course of the experiment with control (Vehicle) animals yielding an average mass increase of 4.6 grams/day whereas treatment groups yielded varying results depending on the route of administration and the active ingredient (FIG. 46). Animals were maintained without treatment until ten weeks of age at which time groups were randomized by weight and blood glucose. Animal weights were recorded daily and expressed here as percent change from Day zero. The rate of weight gain (grams/day) for each group was 4.6, 1.7, 4.0, −1.1, and −3.1 for vehicle, oral berberine, transdermal-berberine, transdermal-base cream, and transdermal-dihydroberberine, respectively.

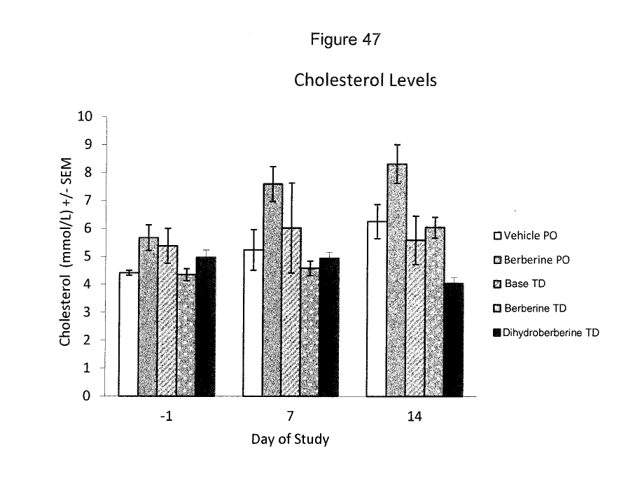

Cholesterol levels were measured for each week of the experiment (FIG. 47). Transdermal-dihydroberberine yielded an overall increase in blood cholesterol. Animals were maintained without treatment until ten weeks of age at which time groups were randomized by weight. Cholesterol levels were recorded weekly for each group. The specific cholesterol levels on Day 14 (mmol/L+/−SEM) were 6.3+/−0.6, 8.3+/−0.7, 5.6+/−0.9, 6.0+/−0.4, and 4.1 +/−0.2 for oral berberine, transdermal-berberine, transdermal-base cream, and transdermal-dihydroberberine, respectively.

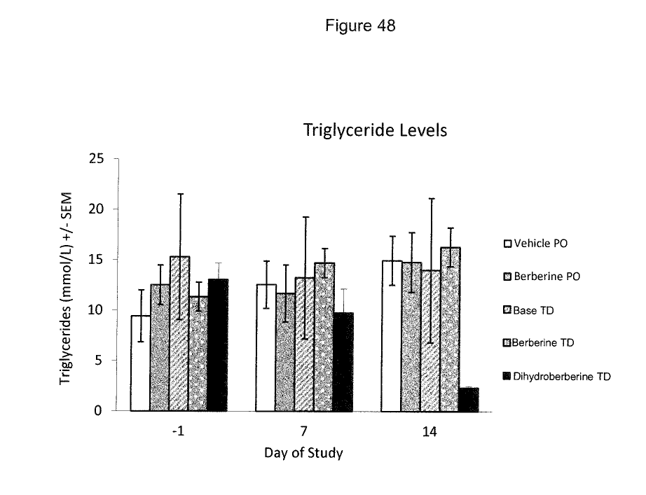

Triglyceride levels were measured for each week of the experiment (FIG. 48). Transdermal-dihydroberberine yielded an overall increase in blood triglyceride levels. Animals were maintained without treatment until ten weeks of age at which time groups were randomized by weight. Cholesterol levels were recorded weekly for each group. The specific triglyceride levels on Day 14 (mmol/L+/−SEM) were 15.0+/−2.4, 14.8+/−3.0, 14.0+/−7.0, 16.3+/−1.9, and 2.3+/−0.2 for oral berberine, transdermal-berberine, transdermal-base cream, and transdermal-dihydroberberine, respectively.

Changes in weight, cholesterol levels and triglycerides levels were observed for the formulation of transdermal-dihydroberberine. This compound and its formulation are prepared with an excipient system that includes isopropyl myristate and polysorbate-20. This compound, while structurally distinct, quickly converts back to berberine rapidly. Overall, this data indicates that a transdermal formulation of dihydroberberine yields efficacious outcomes in a rodent model of metabolic syndrome.

### Example 23: Circulating Levels of Serum Berberine after Chronic Administration by Various Routes and Formats in a Model of Metabolic Syndrome

This study examined the efficacy of administration of berberine and dihydroberberine in transdermal formulations in 24 male Zucker rats (fa/fa), similar to the study protocol in Example 19. The treatment groups are shown in Table 34. The following formulations were prepared:

### Test Article 1

| | |
|---|---|
| Name | Berberine Transdermal (TD) - Formulation 9 |
| Dosage Form | Topical Cream, 5% (w/w) berberine |
| Doses Tested | 3.6 g/kg, BID |
| Lot # | 200416-01 |
| Manufacturer | Delivra Inc. |
| Drug storage during study | Refrigerated 2-4° C. |

### Test Article 2

| | |
|---|---|
| Name | Dihydroberberine Transdermal (TD) - Example 26 |
| Dosage Form | Topical Cream, 5% (w/w) DHB |
| Doses Tested | 3.6 g/kg, BID |
| Lot # | 04152016/04082016 |
| Manufacturer | Delivra Inc. |
| Drug storage during | Refrigerated 2-4° C. |
| study | |

### Positive Control 1

| | |
|---|---|
| Name | Berberine Oral |
| Dosage Form | Powder in Liquid Vehicle |
| Vehicle | 0.5% (w/v) methylcellulose, 0.2% (v/v) |
| | Tween 80 in physiological saline |
| Dose Tested | 180 mg/kg, BID (5 ml/kg) |
| Lot # | BCBL6393V |
| Manufacturer | Sigma Aldrich |
| Drug storage during study | Refrigerated 2-4° C. |

### Negative Control 1

| | |
|---|---|
| Name | Transdermal Base |
| Dosage Form | Topical Cream |
| Dose Tested | 3.6 g/kg, BID |
| Lot # | 04152016P |
| Manufacturer | Delivra Inc. |
| Drug storage during study | Refrigerated 2-4° C. |

### Vehicle

| | |
|---|---|
| Name | 0.5% (w/v) methylcellulose, 0.2% (v/v) |
| | Tween 80 in physiological saline |

### Results

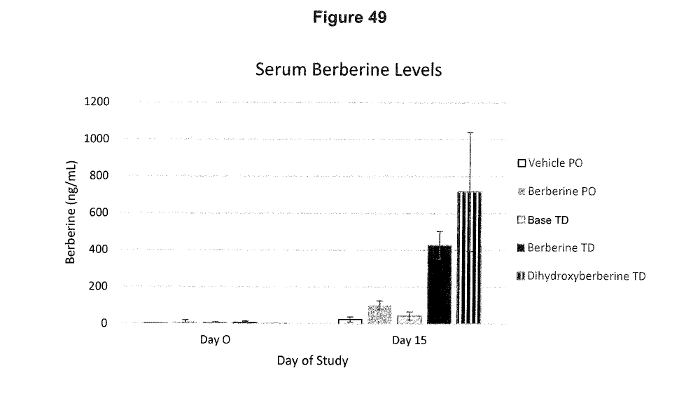

The serum berberine levels of animals involved in the Zucker fa/fa “Steinbeck” experiment were tested at Day zero and fifteen. Concentrations varied from group to group with the highest levels in those animals treated with dihydroberberine. In addition, transdermal berberine outperformed oral berberine as shown in FIG. 49. Plasma concentrations of berberine in rats treated; orally [PO] with Vehicle (0.5% w/v methyl cellulose+0.2% v/v Twee-80 in saline), transdermally [TD] with transdermal Base Cream (3.6 mg/kg/dose), orally with Berberine (180 mg/kg/dose), transdermally with Beberine Cream (3.6 g/kg/dose), transdermally with 5% w/w Dihydroberberine Cream (3.6 g/kg/dose) were analyzed.

The objective of this study was to examine the effect of two routes (oral and transdermal) on the delivery of berberine into systemic circulation and the influence of dihydroberberine transdermal delivery of berberine into systemic circulation. The 15 days oral administration of berberine resulted in berberine plasma concentration of 101 ng/mL, which is lower than that observed for both 15 days transdermally administered berberine and dihydroberberine. The 15 days transdermally administered berberine increases berberine plasma concentration to 426 ng/mL, which is about four times higher when compared to the 101 ng/mL level of berberine observed in the 15 days orally administered berberine.

The 15 days transdermal administration of dihydroberberine resulted in 716 ng/mL systemic concentrations of berberine, which shows that 15 days transdermally administered dihydroberberine increases systemic circulation of berberine to a level higher than that of both the 15 days orally and transdermally administered berberine.

### Example 24: Solubility Testing of Berberine and Dihydroberberine Using Various Solvents

Solubility was determined in a step-wise procedure that involved attempting to dissolve the berberine/DHB in the solvents at relatively high concentrations. If the berberine/DHB did not dissolve, the volume of solvent was increased so as to decrease the concentration of berberine/DHB and repeated in an attempt to solubilize the berberine/DHB at lower concentration. 5.0 mg of berberine/DHB was weighed and placed in an Eppendorf/15 mL conical tube. Then the expected solvent was added to the berberine/DHB. The container was sonicated at RT or at 40C for 30 minutes. The following solvents were tested to dissolve the DHB. Dimethyl sulfoxide (Reagent Grade; BioShop DMS555), Acetone (Caledon laboratory Chemicals 1201-7-40), Anhydrous ethyl alcohol (Commercial Alcohol, Brampton, ON), Water (Ambion), Mineral oil (Sigma-Aldrich M-1180), Oleyl alcohol (Aldrich-369314), Isosorbid-dimethyl ether, 98% (Aldrich 24, 789-8), Hexylene Glycol, Propanediol, Trivalin, Isopropyl myristate (Aldrich 172472; Lot #STBF1206V), and Glycerol tributyrate (Sigma T8626; Lot #BCBQ 7706V). The following solvents were tested for berberine; ethanol, water, Oleyl alcohol, Isosorbid-dimethyl ether, Isopropyl myristate, and Glycerol tributyrate. The solubility was visually checked and state of solubility was recorded.

The solubility of DHB was determined by using various solvents. The results are shown below in Table 35. It was observed that solvent Isosorbid-dimethyl ether has the highest (250 mg/mL) DHB dissolve capability and the lowest was the water.

In case of berberine, six solvents were tested. It was observed that Isosorbid-dimethyl ether, Oley alcohol, Isopropyl myristate, and Glycerol tributyrate has the highest dissolving capability of 250 mg/mL at 40° C., whereas water and ethanol has maximum of 50 mg/mL dissolving capability at 40° C. (Table 36). At room temperature, the dissolving capability of the solvents was less.

The common solvents (those dissolve both berberine and DHB) at high concentration level are listed in Table 37. It was observed that Isosorbid-dimethyl ether has the capability of dissolving both the berberine and dihydroberberine at 250/mL level.

Dissolving capability of the solvents varied on the temperature of the solvents used. Low dissolving was observed at room temperature and the highest was at 40° C. The dihydroberberine was dissolved by solvents at the level from 0.5 to 250 mg/mL, while berberine was dissolved at the level from 25 to 250 mg/mL. Isosorbid-dimethyl ether dissolved both the berberine and dihydroberberine at the maximum dissolving capability level 250 mg/mL. However, this solvent was not the only material demonstrated to produce higher solubility and a more uniform suspension as compared to the typical solubilizer DMSO. Isopropyl myristate, oleoyl alcohol, and glycerol tributyrate yielded homogenous suspensions.

### Example 25: Comparison of BRB Flux Rates Between DHB and BRB Formulations Using PAMPA Assay

The purpose of this study was to establish whether the PAMPA assay is an appropriate in vitro alternative to in vivo animal models to compare formulations of DHB and BRB for their ability to pass berberine through the skin.

Two formulations of DHB and one of BRB were formulated as described below.

Formulation A of Test Article 5% W/W DHB in Transdermal Base Formulation from Example 26

0.5 g (+/−0.01 g) of dihydroberberine was weighed and placed into mortar. 8 g (+/−0.1 g) was weighed into a 20 mL syringe. The rest of the procedure was performed a steady flow of nitrogen at room temperature. The dihydroberberine (DHB) was ground by mortar and pestle for 10 minutes at which point it was a very fine powder/dust. 1.7625 mL of isopropyl myristate (IPM) was added to the mortar with the DHB and the resulting suspension was macerated with the pestle for 5 minutes. The weighed 8 g of the transdermal base formulation was then added to the mortar and the resulting suspension was macerated for 5 minutes with pestle. The formulation was then transferred to 2×20 mL syringes with a spatula. The syringes were equipped with a connector to another 20 mL syringe to prevent the formulation leaking out. The syringe was inverted and allowed any air to the top. The second syringe was disconnected and the air bubbles were then pushed out of the syringes. The second, empty 20 mL syringe was re-connected to the formulation containing syringe via connector. The formulation was pushed back and forth between the syringes 10× each way. The formulation was then stored at 4° C.

Formulation B of Test Article 5% W/W DHB in Transdermal Base Formulation from Example 26

0.5 g (+/−0.01 g) of dihydroberberine was weighed and placed into mortar. 8 g (+/−0.1 g) of transdermal base formulation was weighed into two 20 mL syringes. The rest of the procedure was performed a steady flow of nitrogen at room temperature. The dihydroberberine (DHB) was ground by mortar and pestle for 10 minutes at which point it was a very fine powder/dust. 1.676 mL of isopropyl myristate (IPM) and 0.088 mL of polysorbate 20 was added to the mortar with the DHB and the resulting suspension was macerated with the pestle for 5 minutes. The weighed 8 g of the transdermal base formulation was then added to the mortar and the resulting suspension was macerated for 5 minutes with pestle. The formulation was then transferred to 2×20 mL syringes with a spatula. The syringes were equipped with a connector to another 20 mL syringe to prevent the formulation leaking out. The syringe was inverted and allowed any air to the top. The second syringe was disconnected and the air bubbles were then pushed out of the syringes. The second, empty 20 mL syringe was re-connected to the formulation containing syringe via connector. The formulation was pushed back and forth between the syringes 10× each way. The formulation was then stored at 4° C.

Formulation C: Test Article 5% W/W BRB in Transdermal Base Formulation

The berberine formulation was prepared according to Formulation 9.

Pampa Assay:

The pampa hydration solution was removed from the refrigerator and allowed to come to room temperature for 1 hour. 3.7 mL of the hydration solution then added to each trough in the reservoir plate corresponding to each set of 8 pampa wells to be hydrated. The pampa sandwich was then carefully assembled with the hydration reservoir on the bottom, the pampa plate in the middle and cover on top. The plate was wrapped in parafilm and allowed to hydrate overnight without being moved or disturbed.

Berberine was weighed into a scintillation vial (2-5 mg) and enough MeOH:H₂O (50:50) was added to make a 1 mg/mL solution. The solution was vortexed to aid dissolution. A 50 μg/mL solution of melatonin was prepared by adding 50 μL of the 1 mg/mL solution to 950 μL of MeOH:H₂O (50:50) which was then vortexed for 10 seconds.

A 1 mg/mL solution of chelerythrine chloride (IS) was prepared in 50:50 MeOH:H₂O and the solution was vortexed to aid dissolution. The solution was stored in the freezer (−20° C.) and removed from the freezer and allowed to thaw immediately before use. A g/mL solution of IS was prepared by adding 20 μL of the 1 mg/mL solution to 980 μL of MeOH:H₂O (50:50) which was then vortexed for 10 seconds.

A 125 ng/mL solution of chelerythine chloride was prepared by addition of 625 μL of 20 μg/mL solution of cheleryhtine chloride to approximately 99 mL of 50:50 MeOH:H₂O (measured by graduated cylinder). (Solution A)

A solution of pampa assay buffer was prepared by adding 1.25 mL of Prisma HT buffer to 48.75 mL of distilled water. The pH of the buffer was adjusted to 7.0 with 0.5 M NaOH.

The creams to be tested were first transferred into a 5 mL syringe. As much of the air as possible was pushed from the syringe with the plunger. A second 5 mL syringe was attached to the first syringe via connector. The cream was forced from one syringe to the other until one large bubble containing most of the air was adjacent to the plunger of one of the syringes. Then the cream was pushed into the other syringe leaving the bubble of air in the other syringe. The cream containing syringe was then detached from the 5 mL syringe and attached to a 1 mL syringe. The cream was carefully transferred to the 1 mL syringe until full (overflowing with plunger removed). The plunger was then replaced. A 14 gauge needle was then attached to the end of the syringe, and the plunger was pushed until the cream filled the dead volume of the needle. The plunger was pushed until it reached an even graduation (ex. 1.0 mL). The needle was placed just touching the middle of the bottom of the pampa donor well, and very slowly and carefully not to introduce air pockets, 0.2 mL of the cream was added to the donor well. This was repeated until 8 wells contained the appropriate amount of creams to be tested. The pampa sandwich was then assembled and then 200 μL of prisma buffer was added to each well using a multichannel pipette. 5 μL of the receiver solution was sampled at 1 h, 2 h, 3 h, 4 h an 5 h time points. The 5 μL sample was added to 995 μL of 125 ng/mL solution of chelerythrine chloride (Solution A) in a eppindorf tube. The sample was then vortexed for 15 seconds, before 100 μL of the solution was then transferred to a well in a 96 well plate for HPLC-MS analysis.

HPLC-MS Instrumentation and Conditions:

Isocratic chromatographic separation was performed on a (Zorbax eclipse XDB C18 column (4.6×150 mm, 5 micron particle size Agilent USKH009316) with guard using a mobile phase of MeOH (0.2% formic acid): water (0.4% formic acid) (50:50) at a flow rate of 1 mL/min for 5 min. The first 2 minutes was sent to the waste. There was no post time. The column temperature was 40° C. and the auto sampler temperature was maintained at 5° C. The sample injection volume was 10 μL and the injector is set to bottom sensing enabled. A 5500 Q trap from AB Sciex Instruments equipped with an electrospray ionization (ESI) was used in the positive ion mode with multiple reaction monitoring (MRM) for the quantitative analysis. Nitrogen was used as the collision gas and the curtain gas. The curtain gas was 10.00 psi, the collision gas was set to low, the ion spray voltage was 5500 volts, the temperature was 450° C., and gas sources 1 and 2 were 40 psi. The declustering potential was 110 volts, the exit potential was 4.00 volts, the focusing lens 1 was −10.50 volts and the cell exit potential was 4.00 volts. Quantification was performed using the transitions m/z 336.08->292.1 (CE=45 V, 100 msec) for berberine and 338.08->294.1 (CE=45 V, 100 msec) for chelerythine (IS).

### Standard Series Reproducibility and Linearity

Results

The ratio of peak area of berberine/concentration of berberine did not follow a linear curve in the concentrations tested (FIG. 1). Therefore, the berberine peak area was used as the comparative measure instead of converting to concentration as is customary. The peak areas associated with each of the concentration stocks of berberine were compared for consistency over three days of experiments, the standard error of the mean was calculated for the peak areas for each standard series concentration. The concentrations >7.8 ng/mL had acceptable standard errors across daily experiments (<20). LLOD was <1.9 ng/mL (peak area >3×blank) and LLOQ was <7.8 ng/mL for (berberine peak area >10×blank peak area).

LLOD was <1.9 ng/mL (peak area >3×blank) and LLOQ was <7.8 ng/mL for (berberine peak area >10×blank peak area).

The described PAMPA experiment was repeated on three separate days. The first and second days, 8 replicates per cream were included. On the final day due to limited supply of the test creams, Cream A was performed in 5 replicates and cream B in 7 and cream C, 8. Total number of replicates for each cream over three experiments were 21 (A), 23 (B) and 24 (C). The coefficient of error for the combined data for each cream/time point were calculated and became within the acceptable range (<20) only at and after the 3 hour time point for all samples (Coeff of error range=6.7-16.2).

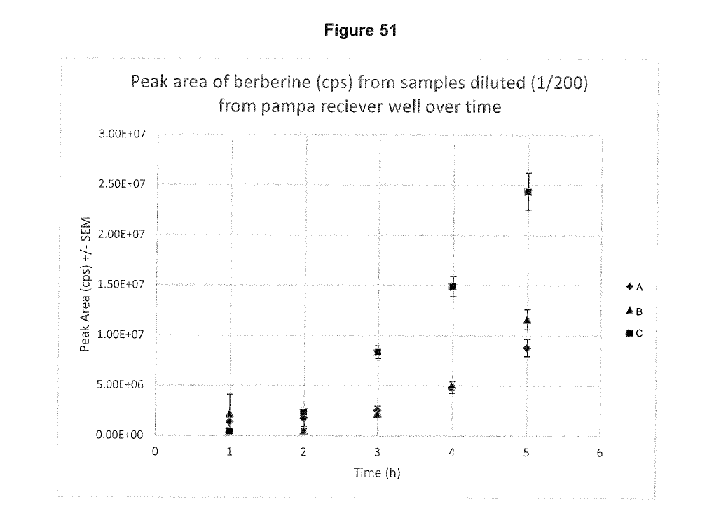

The berberine cream (cream C) consistently displayed a higher flux rate than either of DHB creams (cream A and B), and had a strongly statistically significant (p<0.001) differences in berberine concentrations in the pampa acceptor well than either of the other creams at the 3, 4 and 5 hour time points. The DHB formulation with Tween (formulation B) had a higher concentration of berberine than the formulation A (without tween) at the 5 hour time point (p=0. 0.0422). (FIG. 51). Formulation A of Test Article 5% W/W DHB in transdermal base formulation (80% w/w), isopropyl myristate (15% w/w). 1 h: 1.37 e⁶ CPS+/−6.4 e⁵ (mean CPS+/−SEM), 2 h: 1.76 e⁶ CPS+/−8.21 e⁵ (mean CPS+/−SEM), 3 h: 2.56 e⁶ CPS+/−4.16 e⁵ (mean CPS+/−SEM), 4 h: 4.84 e⁶ CPS+/−5.92 e⁵ (mean CPS+/−SEM), 5 h: 8.78 e⁶ CPS+/−8.54 e⁵ (mean CPS+/−SEM). Formulation B of Test Article 5% W/W DHB in transdermal base formulation (80% w/w), isopropyl myristate (14.25% w/w) and Tween 20 (0.75% w/w) (LOT 04152006). 1 h: 1.37 e⁶ CPS+/−6.4 e⁵ (mean CPS+/−SEM), 2 h, 1.76 e⁶ CPS+/−8.21 e⁵ (mean CPS+/−SEM), 3 h, 2.56 e⁶ CPS+/−4.16 e⁵ (mean CPS+/−SEM), 4 h: 4.84 e⁶ CPS+/−5.92 e⁵ (mean CPS+/−SEM), 5 h, 8.78 e⁶ CPS+/−8.54 e⁵ (mean CPS+/−SEM). Formulation C: Test Article 5% W/W BRB in D transdermal base formulation. 1 h: 1.37 e⁶ CPS+/−6.4 e⁵ (mean CPS+/−SEM), 2 h, 1.76 e⁶ CPS+/−8.21 e⁵ (mean CPS+/−SEM), 3 h, 2.56 e⁶ CPS+/−4.16 e⁵ (mean CPS+/−SEM), 4 h, 4.84 e⁶ CPS+/−5.92 e⁵ (mean CPS+/−SEM), 5 h, 8.78 e⁶ CPS+/−8.54 e (mean CPS+/−SEM).

The results of the pampa assay indicate that the berberine test cream has the highest rate of penetration of berberine through the membrane in the PAMPA out of the three creams tested. It is suggested that the rate limiting factor of the permeation rate of BRB in the experiments with the DHB formulations is the oxidation of DHB to BRB, which must occur before the molecule is soluble in the aqueous buffer of the assay and able to pass through the membrane. This oxidation step is not required in the case of the BRB cream, a possible reason for its higher penetration rate. This is contrast to the animal study, where the DHB is expected to pass through the skin before being oxidized to BRB post absorption. This highlights a fundamental difference in the two methods (in vitro vs in vivo).

The results also indicate that the inclusion of Tween increases the flux rate of berberine slightly in the assay.

The study results show that the BRB cream (formulation C) consistently displays a higher penetration rate of BRB in the PAMPA assay compared to either of the DHB creams, and the tween containing DHB formulation has a higher penetration rate of BRB compared with the one lacking tween.

### Example 26: Topical Formulation 9 Comprising Dihydroberberine

A topical formulation was prepared according to Example 11 (formulation 9) without berberine. In a separate vessel, 6.7 mL of isopropyl myristate and 352 μL of polysorbate 20 were mixed. To this vessel, 2 g of dihydroberberine was added and mixed until a homogeneous suspension was formed. The homogeneous suspension containing the dihydroberberine was then mixed with the topical formulation to obtain a transdermal formulation containing dihydroberberine.

Additional concerns with the study, first that the composition of DHB formulation is not identical to the BRB formulations, which introduces additional variables. Additionally, the sampling in the animal study is after several days of treatment, not hours as is the method of the assay which is not reflective of a direct comparison

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1

| | Material | | Volume required | |
|---|---|---|---|---|
| Solvent | (Berberine) | Mass (μg) | to dissolve (mL) | |
| Trivalin | Commercial | 40 | >2 | mL |
| Hexylene Glycol | Commercial | 35 | >1.5 | mL |
| Propanediol | Commercial | 39 | >5 | mL |
| Arlasolve | Commercial | 39 | >5 | mL |
| Trivalin | Purified | 37 | 1.0 | mL |
| Hexylene Glycol | purified | 36 | 1.0 | mL |
| Propanediol | purified | 39 | 0.6 | mL |
| Arlasolve | purified | 35 | >5 | mL |

TABLE 2
Formulation 1

| | Ingredients | % |
|---|---|---|
| Phase A | Emulsifier | 4% |
| | Polar Emollient Oils | 3-7% |
| | Medium Polar Emollient | 2% |
| Phase B | Water | 50% |
| | Berberine containing | 3% |
| | extract | |
| Phase C | Berberine containing | 6% |
| | extracts | |
| Phase D | Flavonoid containing | 2-8% |
| | extracts | |
| Phase E | Preservatives | 1.6% |
| Phase F | Antioxidant | 0.5% |
| | Solubilizer | 3.5% |
| Phase G | Water | 1.5% |
| | Non-essential amino | 0.5% |
| | acid | |
| Phase H | Phospholipid complexed | 2% |
| | flavonoid | |
| | Water | 6% |
| Phase I | Thickening agent | 2.9% |
| Phase J | Surfactant | 1.5% |
| | Total | 100.00% |

TABLE 3
Formulation 2

| | Ingredients | % |
|---|---|---|
| Phase A | Emulsifier | 4% |
| | Berberine containing | 3% |
| | extract | |
| | Polar Emollient Oils | 3-7% |
| | Medium Polar Emollient | 2% |
| Phase B | Water | 50% |
| | Polyol | 2% |
| Phase C | Berberine containing | 2-4% |
| | extracts | |
| Phase D | Flavonoid containing | 2-8% |
| | extracts | |
| Phase E | Preservatives | 1.6% |
| Phase F | Antioxidant | 0.5% |
| | Solubilizer | 3.5% |
| Phase G | Water | 1.5% |
| | Non-essential amino | 0.5% |
| | acid | |
| Phase H | Phospholipid complexed | 2% |
| | flavonoid | |
| | Water | 6% |
| Phase I | Thickening agent | 2.9% |
| Phase J | Surfactant | 1.5% |
| | Total | 100.00% |

TABLE 4
Formulation 3

| | Ingredients | % |
|---|---|---|
| Phase A | Emulsifier | 4% |
| | Polar Emollient Oils | 3-7% |
| | Medium Polar Emollient | 2% |
| Phase B | Water | 53% |
| | Berberine containing | 10% |
| | extract | |
| Phase C | Flavonoid containing | 2-4% |
| | extracts | |
| Phase D | Preservatives | 1.6% |
| Phase E | Antioxidant | 0.5% |
| | Solubilizer | 3.5% |
| Phase F | Water | 1.5% |
| | Non-essential amino | 0.5% |
| | acid | |
| Phase G | Phospholipid complexed | 2% |
| | flavonoid | |
| | Water | 6% |
| Phase H | Thickening agent | 2.9% |
| Phase I | Surfactant | 1.5% |
| | Total | 100.00% |

TABLE 5
Stability parameters, pH, texture, color and odour
for formulation 3 at 45° C. for 90 days

| | | | Viscosity | | | |
|---|---|---|---|---|---|---|
| | Avg. Temp | | (T4-2 rpm, | | | |
| Months | (° C.) | pH | cps) | Appearance | Colour | Scent |
| 0 | 25 | 4.28 | 18600 | cream | yellow | characteristic |
| 0.5 | 25 | 4.39 | 11220 | cream + oil in | yellow | characteristic |
| | | | | the surface | | |
| 1 | 25 | 4.27 | | cream + oil in | yellow | characteristic |
| | | | | the surface | | |
| 2 | 25 | | | | | |
| 3 | 25 | | | | | |

TABLE 6
Stability parameters, pH, texture, color and odour for formulation
3a at 45° C. for 90 days

| | | | Viscosity | | | |
|---|---|---|---|---|---|---|
| | Avg. Temp | | (T4-2 rpm, | | | |
| Months | (° C.) | pH | cps) | Appearance | Colour | Scent |
| 0 | 25 | 4.40 | 12990 | cream | yellow | characteristic |
| 1 | 25 | 4.34 | | cream + oil in | yellow | characteristic |
| | | | | the surface | | |
| 2 | 25 | | | | | |
| 3 | 25 | | | | | |

TABLE 7
Formulation 4

| | Ingredients | % |
|---|---|---|
| Phase A | Emulsifier | 4% |
| | Polar Emollient Oils | 3-7% |
| | Medium Polar Emollient | 2% |
| Phase B | Water | 38% |
| | Berberine containing | 10% |
| | extract | |
| Phase C | Antioxidant | 5% |
| | Water | 10% |
| Phase D | Flavonoid-containing | 2-4% |
| | extracts | |
| Phase E | Preservatives | 1.6% |
| Phase F | Antioxidant | 0.5% |
| | Solubilizer | 3.5% |
| Phase G | Water | 1.5% |
| | Non-essential amino | 0.5% |
| | acid | |
| Phase H | Phospholipid complexed | 2% |
| | flavonoid | |
| | Water | 6% |
| Phase I | Thickening agent | 2.9% |
| Phase J | Surfactant | 1.5% |
| | Total | 100.00% |

TABLE 8
Stability parameters, pH, texture, color and odour for formulation 4
at 45° C. for 90 days

| | | | Viscosity | | | |
|---|---|---|---|---|---|---|
| | Avg. Temp | | (T4-2 rpm, | | | |
| Months | (° C.) | pH | cps) | Appearance | Colour | Scent |
| 0 | 25 | 4.71 | 13260 | cream | greenish | characteristic |
| | | | | | beige | |
| 1 | 25 | 4.69 | | cream + oil in | greenish | characteristic |
| | | | | the surface | beige | |
| 2 | 25 | | | | | |
| 3 | 25 | | | | | |

TABLE 9
Formulation 5

| | Ingredients | % |
|---|---|---|
| Phase A | Emulsifier | 4.5% |
| | Wax Stabilizer | 1.5% |
| | Polar Emollient Oils | 3-7% |
| | Medium Polar Emollient | 2% |
| Phase B | Water | 45% |
| | Thickening agent | 0.40% |
| | Polyol | 4% |
| Phase C | Preservative booster | 10% |
| | Antioxidant | 0.50% |
| | Berberine containing | 5% |
| | extract | |
| Phase D | Flavonoid-containing | 2-4% |
| | extracts | |
| Phase E | Preservatives | 1.6% |
| Phase F | Antioxidant | 0.5% |
| | Solubilizer | 2.0% |
| Phase G | Water | 1.5% |
| | Non-essential amino | 0.5% |
| | acid | |
| Phase H | Phospholipid complexed | 2% |
| | flavonoid | |
| | Water | 6% |
| Phase I | Thickening agent | 2% |
| | Total | 100.00% |

TABLE 10
Formulation 6

| | Ingredients | % |
|---|---|---|
| Phase A | Emulsifier | 4.5% |
| | Wax Stabilizer | 1.5% |
| | Polar Emollient Oils | 3-7% |
| | Medium Polar Emollient | 2% |
| Phase B | Water | 45% |
| | Thickening agent | 0.40% |
| | Polyol | 4% |
| Phase C | Polyol | 10% |
| | Antioxidant | 0.50% |
| | Berberine containing | 5% |
| | extract | |
| Phase D | Flavonoid-containing | 2-4% |
| | extracts | |
| Phase E | Preservatives | 1.6% |
| Phase F | Antioxidant | 0.5% |
| | Solubilizer | 2.0% |
| Phase G | Water | 1.5% |
| | Non-essential amino | 0.5% |
| | acid | |
| Phase H | Phospholipid complexed | 2% |
| | flavonoid | |
| | Water | 6% |
| Phase I | Thickening agent | 2% |
| | Total | 100.00% |

TABLE 11
Stability parameters, pH, texture, color and odour for formulation 6
at 45° C. for 90 days

| | | | Viscosity | | | |
|---|---|---|---|---|---|---|
| | Avg. Temp | | (T4-2 rpm, | | | |
| Months | (° C.) | pH | cps) | Appearance | Colour | Scent |
| 0 | 25 | 4.28 | NA | cream | yellow | characteristic |
| 0.5 | 25 | 3.59 | NA | cream + brown | yellow + | characteristic |
| | | | | oil | dark | |
| | | | | | greenish | |
| | | | | | brown | |
| 1 | 25 | | | | | |
| 2 | 25 | | | | | |
| 3 | 25 | | | | | |

TABLE 12
Formulation 7

| | Ingredients | % |
|---|---|---|
| Phase A | Emulsifier | 4.5% |
| | Wax Stabilizer | 1.5% |
| | Polar Emollient Oils | 3-7% |
| | Medium Polar Emollient | 2% |
| Phase B | Water | 45% |
| | Thickening agent | 0.40% |
| | Polyol | 4% |
| Phase C | Preservative booster | 10% |
| | Berberine containing | 5% |
| | extract | |
| Phase D | Flavonoid-containing | 2-4% |
| | extracts | |
| Phase E | Preservatives | 1.6% |
| Phase F | Antioxidant | 0.5% |
| | Solubilizer | 2.5% |
| Phase G | Water | 3.0% |
| | Non-essential amino | 0.5% |
| | acid | |
| Phase H | Phospholipid complexed | 2% |
| | flavonoid | |
| | Water | 6% |
| Phase I | Thickening agent | 0.5% |
| | Total | 100.00% |

TABLE 13
The stability parameters, pH, texture, color and odour for formulation
7 at 45° C. for 90 days

| | | | Viscosity | | | |
|---|---|---|---|---|---|---|
| | Avg. Temp | | (T4-2 rpm, | | | |
| Months | (° C.) | pH | cps) | Appearance | Colour | Scent |
| 0 | 25 | 4.26 | 14220 | cream | yellow | characteristic |
| 1 | 25 | 3.99 | 19760 | cream + oil | yellow | characteristic |
| | | | | drops | | |
| 2 | 25 | 3.86 | 19110 | cream + oil | yellow | characteristic |
| 3 | 25 | 3.83 | 27150 | cream + oil | yellow | characteristic |

TABLE 14
Formulation 8

| | Ingredients | % |
|---|---|---|
| Phase A | Emulsifier | 4.5% |
| | Wax Stabilizer | 1.5% |
| | Polar Emollient Oils | 3-7% |
| | Medium Polar Emollient | 2% |
| Phase B | Water | 45% |
| | Thickening agent | 0.40% |
| | Polyol | 4% |
| Phase C | Preservative booster | 10% |
| | Antioxidant | 0.5% |
| | Tetrahydroberberine | 5% |
| | containing extract | |
| Phase D | Flavonoid-containing | 2-4% |
| | extracts | |
| Phase E | Preservatives | 1.6% |
| Phase F | Antioxidant | 0.5% |
| | Solubilizer | 2% |
| Phase G | Water | 1.5% |
| | Non-essential amino | 0.5% |
| | acid | |
| Phase H | Phospholipid complexed | 2% |
| | flavonoid | |
| | Water | 6% |
| Phase I | Thickening agent | 2% |
| | Total | 100.00% |

TABLE 15
The stability parameters, pH, texture, color and odour for formulation
8 at 45° C. for 90 days

| | | | Viscosity | | | |
|---|---|---|---|---|---|---|
| | Avg. Temp | | (T4-2 rpm, | | | |
| Months | (° C.) | pH | cps) | Appearance | Colour | Scent |
| 0 | 25 | 4.35 | 37260 | cream | yellow | characteristic |
| 1 | 25 | 4.53 | 55140 | cream | yellow | characteristic |
| 2 | 25 | 4.53 | 51540 | cream | yellow + | characteristic |
| | | | | | dark brown | |
| 3 | 25 | 4.30 | 34500 | cream + oil | yellow + | characteristic |
| | | | | drop | dark brown | |

TABLE 16
Formulation 9

| | Ingredients | % |
|---|---|---|
| Phase A | Emulsifier | 4.5% |
| | Wax Stabilizer | 1.5% |
| | Polar Emollient Oils | 3-7% |
| | Medium Polar Emollient | 2% |
| Phase B | Water | 32% |
| | Thickening agent | 0.40% |
| | Polyol | 4% |
| Phase C | Polyol | 10% |
| | Water | 10% |
| | Alcohol | 5% |
| | Berberine hydrochloride | 5% |
| Phase D | Flavonoid-containing | 2-4% |
| | extracts | |
| Phase E | Preservatives | 1.6% |
| Phase F | Antioxidant | 0.5% |
| | Solubilizer | 2% |
| Phase G | Water | 1.5% |
| | Non-essential amino | 0.5% |
| | acid | |
| Phase H | Phospholipid complexed | 2% |
| | flavonoid | |
| | Water | 6% |
| Phase I | Thickening agent | 0.5% |
| | Total | 100.00% |

TABLE 17
Formulation comparison in a study in Sprague-Dawley rats.

| Formulation | | | Serum Concentration |
|---|---|---|---|
| Code | Formulation | Time (hours) | (ng/mL) |
| Berberine Lot: 13- | 3 | 0 | 0.46 |
| 557-337 | | 2 | 1.40 |
| | | 4 | 0.46 |
| | | 6 | 0.45 |
| Berberine Lot: 13- | 3a | 0 | 0.45 |
| 558-338 | | 2 | 1.24 |
| | | 4 | 0.49 |
| | | 6 | 0.46 |
| Berberine Lot: 13- | 4 | 0 | 0.45 |
| 559-338 | | 2 | 2.23 |
| | | 4 | 0.46 |
| | | 6 | 0.45 |

TABLE 18
Individualized data for the pharmacokinetics of berberine in serum
with multiple routes of administration

| | | Animals | | | | | | | | Average Concentration | Standard | Standard Error |
|---|---|---|---|---|---|---|---|---|---|---|---|---|
| Treatment | Time (hours) | 1 | 2 | 3 | 4 | 5 | 6 | 7 | 8 | (ng/mL) | Deviation | of the Mean |
| ORAL | 0 | 0.00 | 0.00 | 1.19 | | | | | | 0.40 | 0.69 | 0.40 |
| | 0.5 | 0.12 | 0.00 | 1.99 | | | | | | 0.70 | 1.11 | 0.64 |
| | 1 | 0.03 | 0.00 | 1.72 | | | | | | 0.58 | 0.98 | 0.57 |
| | 1.5 | 0.02 | 0.21 | 5.21 | | | | | | 1.81 | 2.94 | 1.70 |
| | 2 | 0.78 | 0.00 | 0.32 | | | | | | 0.37 | 0.39 | 0.23 |
| | 3 | 0.91 | 6.55 | 6.66 | | | | | | 4.71 | 3.29 | 1.90 |
| | 4 | 0.42 | 0.26 | 0.61 | | | | | | 0.43 | 0.18 | 0.10 |
| | 5 | 1.07 | 5.45 | 0.64 | | | | | | 2.39 | 2.66 | 1.54 |
| PLO | 0 | 0.09 | 1.18 | 0.06 | 0.00 | 0.00 | 0.00 | 0.00 | 0.00 | 0.17 | 0.41 | 0.15 |
| | 0.5 | 0.98 | 8.77 | 1.93 | 0.00 | 0.00 | 0.00 | 0.00 | 0.00 | 1.46 | 3.04 | 1.07 |
| | 1 | 1.83 | 2.45 | 0.39 | 0.00 | 0.00 | 0.00 | 0.00 | 0.00 | 0.58 | 0.99 | 0.35 |
| | 1.5 | 1.87 | 1.54 | 7.66 | 0.15 | 0.00 | 0.00 | 0.00 | 0.00 | 1.40 | 2.64 | 0.93 |
| | 2 | 0.00 | 4.00 | 0.38 | 1.23 | 0.00 | 0.00 | 0.00 | 14.93 | 2.57 | 5.18 | 1.83 |
| | 3 | 0.00 | 2.75 | 7.18 | 2.29 | 11.51 | 0.00 | 0.00 | 0.00 | 2.97 | 4.25 | 1.50 |
| | 4 | 3.77 | 2.50 | 4.74 | 3.24 | 0.00 | 0.00 | 0.00 | 0.00 | 1.78 | 2.00 | 0.71 |
| | 5 | 0.08 | 2.03 | 2.86 | 0.26 | 4.19 | 0.00 | 0.00 | 1.65 | 1.38 | 1.57 | 0.56 |
| Formulation 9 | 0 | 0.00 | 0.29 | 2.95 | 0.16 | 0.00 | 0.00 | 0.00 | 0.00 | 0.42 | 1.02 | 0.36 |
| | 0.5 | 0.58 | 0.00 | 2.69 | 8.42 | 0.00 | 0.00 | 0.00 | 27.52 | 4.90 | 9.59 | 3.39 |
| | 1 | 3.10 | 0.00 | 4.22 | 3.39 | 3.87 | 0.00 | 0.00 | 0.00 | 1.82 | 1.98 | 0.70 |
| | 1.5 | 0.60 | 3.77 | 1.76 | 1.85 | 1.46 | 1.09 | 14.64 | 0.00 | 3.15 | 4.78 | 1.69 |
| | 2 | 16.00 | 1.73 | 1.46 | 0.17 | 0.00 | 18.95 | 296.57 | 0.00 | 41.86 | 103.20 | 36.49 |
| | 3 | 144.62 | 12.65 | 2.70 | 1.87 | 169.17 | 29.77 | 1.48 | 0.00 | 45.28 | 69.89 | 24.71 |
| | 4 | 19.18 | 10.37 | 2.77 | 0.86 | 0.19 | 2.28 | 0.00 | 204.01 | 29.96 | 70.64 | 24.97 |
| | 5 | 2.29 | 0.42 | 3.22 | 1.32 | 0.00 | 0.00 | 15.74 | 0.53 | 2.94 | 5.30 | 1.87 |

TABLE 19
List of Treatments used in Example 17

| | | | | Serum |
|---|---|---|---|---|
| Group | Treatment | Delivery | Dose | Collection |
| A | Simvastatin | Oral | 6 mg/kg/dose | On 28 days |
| B | Berberine | Oral | 180 mg/kg/dose | On 28 days |
| C | Metformin | Oral | 200 mg/kg/dose | On 28 days |
| D | Vehicle | Oral | | On 28 days |
| E | Exemplary | Transdermal | 3.6 g/kg/dose | On 28 days |
| | Formulation 9 | | | |
| F | Exemplary | Transdermal | TD 3.6 g/kg/dose | On 28 days |
| | Formulation 9 | (TD) and | and | |
| | (TD) and | Oral (PO) | PO 6 mg/kg/dose | |
| | Simvastatin | | | |
| | (PO) | | | |
| G | Exemplary | Transdermal | TD 3.6 g/kg/dose | On 28 days |
| | Formulation 9 | (TD) and | and | |
| | (TD) and | Oral (PO) | PO 200 g/kg/dose | |
| | Metformin | | | |
| | (PO) | | | |

TABLE 20
PCSK9 Concentrations for Example 17

| | | | | Average | |
|---|---|---|---|---|---|
| | | PCSK9 | | PCSK9 | Standard |
| | | Concentration | | Concentration | Deviation |
| Group | Treatment | (ng/mL) | | (ng/mL) | (std) |
| A | Simvastatin (oral) | 7310 | 287.5 | 3798.75 | 4965.7 |
| B | Berberine (oral) | 257.5 | 260 | 258.75 | 1.7678 |
| C | Metformin (oral) | 727.5 | 322.5 | 525 | 286.38 |
| D | Vehicle | 265 | 0 | 132.5 | 187.38 |
| E | Exemplary | 0 | 0 | 0 | 0 |
| | Formulation 9 | | | | |
| | (transdermal) | | | | |
| F | Exemplary | 0 | 247.5 | 123.75 | 175.01 |
| | Formulation 9 | | | | |
| | (transdermal) and | | | | |
| | Simvastatin (oral) | | | | |
| G | Exemplary | 327.5 | 337.5 | 332.5 | 7.0711 |
| | Formulation 9 | | | | |
| | (transdermal) and | | | | |
| | Metformin (oral) | | | | |

TABLE 32
Dosing Chart for Example 19

| Group | Test Article & Dose | No. of | Route & | PO Dose |
|---|---|---|---|---|
| ID | Level | Subjects | Frequency | Volume |
| A | Simvastatin | 2 | PO BID | 5 ml/kg |
| | (6 mg/kg/dose) | | | |
| B | Berberine | 2 | PO BID | 5 ml/kg |
| | (180 mg/kg/dose) | | | |
| C | Metformin | 2 | PO BID | 5 ml/kg |
| | (200 mg/kg/dose) | | | |
| D | Vehicle 0.5% (w/v) | 2 | PO BID | 5 ml/kg |
| | methylcellulose, 0.2% | | | |
| | (v/v) Tween 80) | | | |
| E | Berberine | 2 | TD BID | n/a |
| | (3.6 g/kg/dose) | | | |
| F | Berberine | 2 | TD (Berberine) | 5 ml/kg |
| | (3.6 g/kg/dose) + | | PO | |
| | Simvastatin | | (Simvastatin) | |
| | (6 mg/kg/dose) | | BID | |
| G | Berberine | 2 | TD (Berberine) | 5 ml/kg |
| | (3.6 g/kg/dose) + | | PO (Metformin) | |
| | Metformin | | BID | |
| | (200 mg/kg/dose) | | | |

TABLE 33
Treatment Groups for Example 22

| | | | | | Number |
|---|---|---|---|---|---|
| | | | | | of |
| Group | Treatment | Route | Dose | Frequency | Animals |
| A | Vehicle | Oral Gavage | 5 ml/kg | BID | 3 |
| B | Transdermal | Transdermal/ | 3.6 g/kg | BID | 3 |
| | Base (no | Topical | | | |
| | active) | | | | |
| C | Berberine | Oral Gavage | 180 mg/kg | BID | 6 |
| | | | (5 ml/kg) | | |
| D | Berberine | Transdermal/ | 3.6 g/kg | BID | 6 |
| | | Topical | | | |
| E | Dihydro- | Transdermal/ | 3.6 g/kg | BID | 6 |
| | berberine | Topical | | | |

TABLE 34
Treatment Groups

| | | | | | Number of |
|---|---|---|---|---|---|
| Group | Treatment | Route | Dose | Frequency | Animals |
| A | Vehicle | Oral Gavage | 5 ml/kg | BID | 3 |
| B | Transdermal | Transdermal/ | 3.6 g/kg | BID | 3 |
| | Base | Topical | | | |
| C | Berberine | Oral Gavage | 180 mg/kg | BID | 6 |
| | | | (5 ml/kg) | | |
| D | Berberine | Transdermal/ | 3.6 g/kg | BID | 6 |
| | | Topical | | | |
| E | Dihydro- | Transdermal/ | 3.6 g/kg | BID | 6 |
| | berberine | Topical | | | |

TABLE 35
Solubility testing of dihydroberberine

| | Dissolving | | | |
|---|---|---|---|---|
| | Capability | State of | Sonication | |
| Solvent Used | (mg/mL) | Solubility | Temperature | Final Color |
| Dimethyl | 25.0 | Complete | RT | Dark yellow |
| sulfoxide | 50.0 | Complete | 40 C. | Dark yellow |
| (DMSO) | | | | |
| Ethanol | 1.0 | Complete | RT | Yellow |
| | 2.5 | Complete | 40 C. | Yellow |
| Water | 0.5 | Suspension | RT | Cloudy |
| | | | | yellowish |
| | 1.0 | Suspension | 40 C. | Cloudy |
| | | | | yellowish |
| Acetone | 12.5 | Complete | RT | Yellow |
| | 25.0 | Complete | 40 C. | Dark yellow |
| Mineral oil | 2.0 | Suspension | RT | Cloudy |
| | | | | yellowish |
| | 4.0 | Suspension | 40 C. | Cloudy |
| | | | | yellowish |
| Oley alcohol | 12.5 | Suspension | RT | Bright yellow |
| | 50.0 | Suspension | 40 C. | Bright yellow |
| Isosorbid- | 25.0 | Complete | RT | Yellow |
| dimethyl | 100.0 | Complete | 40 C. | Dark yellow |
| ether 98% | 250.0 | Suspension | 40 C. | Brownish yellow |
| Hexylene | 0.5 | Complete | RT | Yellow |
| glycol | 1.0 | Complete | 40 C. | Yellow |
| Propanediol | 1.0 | Complete | RT | Light yellow |
| | 2.5 | Complete | 40 C. | Light yellow |
| Trivalin | 2.5 | Complete | RT | Yellow |
| | 5.0 | Complete | 40 C. | Yellow |
| Isopropyl | 10 | Suspension | RT | Yellow |
| myristate | 75-250 | Suspension | 40 C. | Dark yellow |
| Glycerol | 10 | Complete | RT | Light yellow |
| tributyrate | 75-250 | Suspension | 40 C. | Dark yellow |

TABLE 36
Solubility testing of berberine with selected solvents

| | Dissolving | | | |
|---|---|---|---|---|
| | Capability | State of | Sonication | |
| Solvent Used | (mg/mL) | Solubility | Temperature | Final Color |
| Isosorbid- | 50 | Suspension | RT | Yellow |
| dimethyl ether | 250 | Suspension | 40 C. | Dark yellow |
| 98% | | | | |
| Oleyl alcohol | 50 | Suspension | RT | Yellow |
| | 250 | Suspension | 40 C. | Yellow |
| Isopropyl | 50 | Suspension | RT | Yellow |
| myristate | 250 | Suspension | 40 C. | Yellow |
| Glycerol | 50 | Suspension | RT | Yellow |
| tributyrate | 250 | Suspension | 40 C. | Yellow |
| Water | 25 | Suspension | RT | Yellow |
| | 50 | Suspension | 40 C. | Yellow |
| Ethanol | 25 | Suspension | RT | Yellow |
| | 40 | Suspension | 40 C. | Yellow |

TABLE 37
Comparison of solubility of berberine and dihydroberberine for
selected solvents at 40° C.

| | Dissolving | | | |
|---|---|---|---|---|
| | capability (mg/mL) | | | |
| | | Dihydro- | State of solubility | |
| Solvent | Berberine | berberine | Berberine | Dihydroberberine |
| Isosorbid- | 250 | 250 | Suspension | Suspension |
| dimethyl ether | | | | |
| Oley alcohol | 250 | 50 | Suspension | Suspension |
| Isopropyl | 250 | 75-250 | Suspension | Suspension |
| myristate | | | | |
| Glycerol | 250 | 75-250 | Suspension | Suspension |
| tributyrate | | | | |

### REFERENCES

Anderson, T. J., Gregoire, J., Hegele, R. A., Couture, P., Mancini, G. B. J., McPherson, R., Ur, E. (2013). 2012 Update of the Canadian cardiovascular society guidelines for the diagnosis and treatment of dyslipidemia for the prevention of cardiovascular disease in the adult. Canadian Journal of Cardiology, 29, 151-167.
Arrigo, F. G., Cicero, L. C., Rovati, I. S. (2007). Eulipidemic effects of berberine administered alone or in combination with other natural cholesterol-lowering agents. Arzneimittelforschung, 57(1), 26-30.
Genest, J., McPherson, R., Frohlich, J., Anderson, T., Campbell, N., Carpentier, A., Ur, E. (2009). 2009 Canadian cardiovascular society/Canadian guidelines for the diagnosis and treatment of dyslipidemia and prevention of cardiovascular disease in the adult—2009 recommendations. The Canadian Journal of Cardiology, 25(10), 567-579.
Gu, S., Cao, B., Sun, R., Tang, Y., Paletta, J. L. (2014). Molecular biosystems mechanism underlying the lipid-lowering effect of. Molecular Biosystems.
Liu, H., Wang, L., Li, Y., Liu, J., An, M., Zhu, S., Cao, Y., Jiang, Z., Zhao, M., Cai, Z., Dai, L., Ni, T., Liu, W., Chen, S., Wei, C., Zang, C., Shujuan Tian, J. Y. (2014). Structural optimization of berberine as a synergist to restore antifungal activity of fluconazole against drug-resistant candida albicans. Chem Med Chem, 1(9), 207-216.
Kong, W., Wei, J., Abidi, P., Lin, M., Inaba, S., Li, C., Wang, Y., Wang Z., Si, S., Pan, H., Wang, S., Wu, J., Wang, Y., Li, Z., Liu, J., J. J. (2004). Berberine is a novel cholesterol-lowering drug working through a unique mechanism distinct from statins. Nature Medicine, 12(10), 1344-1351.
Kong, W. J., Wei, J., Zuo, Z. Y., Wang, Y. M., Song, D. Q., You, X. F., Jiang, J. D. (2008). Combination of simvastatin with berberine improves the lipid-lowering efficacy. Metabolism: Clinical and Experimental, 57, 1029-1037.
Malenda, A., Skrobanska, A., Issat, T., Winiarska, M., Bil, J., Oleszcsak, B., Nowis, D. A. (2012). Statins impair glucose uptake in tumor cells. Neoplasia, 14, 311-323.
Ms, B., Goldstein, J. L. (2006). Biomedicine. Lowering LDL—not only how low, but how long? Science, 311 (5768), 1721-1723.
Nechepurenko, I. V. (2010). Berberine: Chemistry and biological activity, 18, 1-23.
Parker, B. A., Capizzi, J. A., Grimaldi, A. S., Clarkson, P. M., Cole, S. M., Keadle, J., Thompson, P. D. (2013). Effects of statins on skeletal muscle function. Circulation, 127, 96-103.
Pereira, C. V., Machado, N. G., Oliveira, P. J. (2008). Mechanisms of berberine (Natural Yellow 18)—Induced mitochondrial dysfunction: Interaction with the adenine nucleotide translocator. Toxicological Sciences, 105(2), 408-4017. Prausnitz, M. R., Langer, R. “Transdermal Drug Delivery. Nat. Biotechnol. 2008, 26:1261-1268.
Respiratory, I. M., Turner, N., Li, J., Gosby, A., To, S. W. C., Cheng, Z., Li, J. (2008). A mechanism for the action of berberine to activate. 57.
Timarco, B., Benvenuti, C., Rozza, F., Cimmino, C. S., Giudice, R., Crispo, S. (2011). Clinical evidence of efficacy of red yeast rice and berberine in a large controlled study versus diet. Mediterranean Journal of Nutrition and Metabolism, 4, 133-139.
Zeiger, E. (n.d.). Goldenseal (Hydrastis Canadensis L.) and two of its constituent alkaloids berberine and hydrastine review of toxicological literature, (November 1997).
Zuo, F., Nakamura, N., Akao, T., Hattori, M. (2006). Pharmacokinetics of berberine and its main metabolites in conventional and pseudo germ-free rats determined by liquid chromatography/ion trap mass spectrometry. Drug Metabolism and Disposition. 34(12), 2064-2072.

1. A transdermal formulation comprising,
(a) an aqueous phase comprising water and at least one water soluble emulsion stabilizer;
(b) an oil phase comprising at least one emulsifier, at least one oil soluble emulsion stabilizer, at least one emollient comprising at least one flavonoid and at least one other emollient;
wherein the oil and aqueous phase form an emulsion;
(c) an external phase comprising at least one flavonoid containing-extract, at least one phospholipid-complexed flavonoid and at least one source of berberine or analog or derivative thereof; and optionally
(d) at least one preservative phase.

2. The transdermal formulation of claim 1, further comprising antioxidants and/or reducing agents.

3. The transdermal formulation of claim 2, wherein the antioxidants are selected from one or more of vitamins, extracted polyphenols and non-essential amino acids.

4. The transdermal formulation of claim 1, wherein the source of berberine or analog thereof is selected from one or more of barberry extract, meadow rue, celandine, Berberis aquifolium, Berberis vulgaris, Hydrastis Canadensis, Xanthorhiza simplicissima, Phellodendron amurense californica and Mahonia aquifolium.

5. The transdermal formulation of claim 4, wherein the source of berberine is an extract from Berberis vulgaris, an extract from Hydrastis Canadensis, or an extract from Mahonia aquifolium.

6. (canceled)

7. (canceled)

8. The transdermal formulation of claim 1, wherein the source of berberine or analog thereof is present in the formulation in an amount of about 1% wt % to about 20 wt %, or about 3 wt % to about 15 wt %, of the total formulation.

9. The transdermal formulation of claim 1, wherein the berberine analog or derivative is selected from one or more of berberine, berberine sulfate, berberine bisulfate, berberine hemisulfate, berberine chloride, jatrorrhizine, palmatine, coptisine, 8-ethyl-12-bromoberberine, 8-ethylberberine, 8-methoxyberberine, 8-methylberberine, 8-n-butyl-12-bromoberberine, 8-n-butylberberine, 8-n-hexyl-12-bromoberberine, 8-n-propyl-12-bromoberberine, 8-n-propylberberine, 8-phenyl-12-bromoberberine, 8-phenylberberine, 9-O-acetylberberrubine, 9-O-benzoylberberrubine, 9-O-ethylberberrubine, 9-O-valerylberberrubine, 9-demethylberberine, 9-demethylpalmatine, 9-O-ethyl-berberrubine, 9-O-ethyl-13-ethylberberrubine, 9-lauroylberberrubine chloride, 12-bromoberrubine, 13-ethoxyberberine, 13-ethylberberine, 13-ethylpalmatine, 13-hydroxyberberine, 13-methoxyberberine, 13-methylberberine, 13-methylberberrubine, 13-methyldihydroberberine N-methyl salt, 13-methylpalmatine, 13-n-butylberberine, 13-n-butylpalmatine, 13-n-hexylberberine, 13-n-hexylpalmatine, 13-n-propylberberine, 13-n-propylpalmatine, palmatrubine, dihydroberberines and tetrahydroberberines.

10. The transdermal formulation of claim 9, wherein the berberine analog is dihydroberberine or tetrahydroberberine.

11. The transdermal formulation of claim 1, further comprising one or more statins.

12. The transdermal formulation of claim 11, wherein the one or more statins are selected from atorvastatin, fluvastatin, lovastatin, pitavastatin, pravastatin, rosuvastatin and simvastatin.

13. The transdermal formulation of claim 1 in the form of a cream, gel, liquid suspension, ointment, solution or patch.

14. (canceled)

15. The transdermal formulation of claim 13, wherein the cream has a viscosity of about 50000 cps to about 500000 cps, or about 85000 cps to about 200000 cps as measured using a Brookfield RVT T4 2 RPM instrument at room temperature.

16. A transdermal formulation comprising,
(a) an aqueous phase comprising water and at least one water soluble emulsion stabilizer;
(b) an oil phase comprising at least one emulsifier, at least one oil soluble emulsion stabilizer, at least one emollient comprising at least one flavonoid and at least one other emollient;
wherein the oil and aqueous phase form an emulsion;
(c) an external phase comprising at least one flavonoid containing-extract, at least one phospholipid-complexed flavonoid; and optionally at least one preservative phase; and
(d) a dihydroberberine phase comprising at least one emulsifier, at least one surfactant and dihydroberberine.

17. The transdermal formulation of claim 16, wherein the dihydroberberine phase comprises dihydroberberine, isopropyl myristate and polysorbate 20.

18. A method for the transdermal administration of one or more berberine or analog or derivative thereof comprising administering an effective amount of one or more of the formulations of claim 1 to a subject in need thereof, wherein the one or more formulations comprise the one or more sources of berberine or analog or derivative thereof.

19. A method for treating a berberine-responsive disease or condition comprising administering an effective amount of one or more of the transdermal formulations of claim 1 to a subject in need thereof.

20. The method of claim 19, wherein the berberine-responsive disease or condition is selected from one or more of Type I diabetes, Type 2 diabetes, pre-type I diabetes, pre-type 2 diabetes, hyperlipidemia, pre-hyperlipidemia, heart disease, inflammatory disease, skin disease, metabolic disease, neurological disease, and cancer, wherein the cancer is selected from hepatoma, colon cancer, lung cancer, breast cancer and leukemia.

21. (canceled)

22. The method of claim 20, wherein the berberine-responsive disease or condition is selected from hyperlipidemia and pre-hyperlipidemia, further comprising administering an effective amount of one or more statins and one or more transdermal formulations of claim 1 to a subject in need thereof.

23. (canceled)

24. The method of claim 20, wherein the berberine-responsive disease or condition is selected from type 2 diabetes and pre-type 2 diabetes, further comprising administering an effective amount of one or more glucose regulating compounds and one or more transdermal formulations of claim 1 to a subject in need thereof.

25. (canceled)

26. The method of claim 24, wherein the one or more glucose regulating compounds are selected from metformin and glyburide.
